SEQUENCE LISTING (21 sequences)

SEQ ID NO: 1 - protein, 6 aa, Artificial Sequence
  Description of Artificial Sequence Synthetic peptide
  C-term NH(CH2C6F5)
AKLTGF

SEQ ID NO: 2 - protein, 10 aa, Artificial Sequence
  Description of Artificial Sequence Synthetic peptide
VTLPSTFGAS

SEQ ID NO: 3 - protein, 6 aa, Artificial Sequence
  Description of Artificial Sequence Synthetic peptide
  MOD_RES (1)..(1): Gamma-Glu
ECGPLL

SEQ ID NO: 4 - protein, 6 aa, Artificial Sequence
  Description of Artificial Sequence Synthetic peptide
  MOD_RES (1)..(1): Gamma-Glu
  MOD_RES (2)..(2): CysTol
ECGPLL

SEQ ID NO: 5 - protein, 14 aa, Artificial Sequence
  Description of Artificial Sequence Synthetic peptide
  C-term NH2
FRSNLYGCEKHKAT

SEQ ID NO: 6 - protein, 130 aa, Artificial Sequence
  Description of Artificial Sequence Synthetic polypeptide
GGCGGSDLGKKLLEAARAGQDDEVRILMANGADVNAYDDNGVTPLHLAAFLGHLEIVEVLLKYGADVNAADSWGTTPLHLAATWGHLEIVEVLLKHGADVNAQDKFGKTAFDISIDNGNEDLAEILQKLN

SEQ ID NO: 7 - protein, 17 aa, Artificial Sequence
  Description of Artificial Sequence Synthetic peptide
RSNFYLGCAGLAHDKAT

SEQ ID NO: 8 - protein, 15 aa, Artificial Sequence
  Description of Artificial Sequence Synthetic peptide
IKFTNCGLLCYESKR

SEQ ID NO: 9 - protein, 15 aa, Artificial Sequence
  Description of Artificial Sequence Synthetic peptide
  MOD_RES (6)..(10): Stapled residues
IKFTNCGLLCYESKR

SEQ ID NO: 10 - protein, 17 aa, Artificial Sequence
  Description of Artificial Sequence Synthetic peptide
RSNFYLGCAGLAHDKAT

SEQ ID NO: 11 - protein, 17 aa, Artificial Sequence
  Description of Artificial Sequence Synthetic peptide
RSNFYLGSAGLAHDKAT

SEQ ID NO: 12 - protein, 11 aa, Artificial Sequence
  Description of Artificial Sequence Synthetic peptide
RSNFFLGCAGA

SEQ ID NO: 13 - protein, 15 aa, Artificial Sequence
  Description of Artificial Sequence Synthetic peptide
IKFTNCGLLCYESKR

SEQ ID NO: 14 - protein, 130 aa, Artificial Sequence
  Description of Artificial Sequence Synthetic polypeptide
GGGGGSDLGKKLLEAARAGQDDEVRILMANGADVNAYDDNGVTPLHLAAFLGHLEIVEVLLKYGADVNAADSWGTTPLHLAATWGHLEIVEVLLKHGADVNAQDKFGKTAFDISIDNGNEDLAEILQKLN

SEQ ID NO: 15 - protein, 102 aa, Artificial Sequence
  Description of Artificial Sequence Synthetic polypeptide
SVSDVPRDLEVVAATPTSLLISWDAPAVTVRYYRITYGETGGNSPVQEFTVPGSKSTATISGLKPGVDYTITVYAVTLPSTCGASSKPISINYRTEIDKPSQ

SEQ ID NO: 16 - protein, 101 aa, Artificial Sequence
  Description of Artificial Sequence Synthetic polypeptide
VSDVPRDLEVVAATPTSLLISWDAPAVTVRYYRITYGETGGNSPVQEFTVPGSKSTATISGLKPGVDYTITVYAVTLPSTGGASSKPISINYRTEIDKPSQ

SEQ ID NO: 17 - protein, 63 aa, Artificial Sequence
  Description of Artificial Sequence Synthetic polypeptide
GGGGGVDNKFNKEQQNAFYEILHLPNLNEEQRNAFIQSLKDDPSQSANLLAEAKKLNDACAPK

SEQ ID NO: 18 - protein, 63 aa, Artificial Sequence
  Description of Artificial Sequence Synthetic polypeptide
GGGGGVDNKFNKEQQNAFYEILHLPNLNEEQRNAFIQSLKDDPSQSANLLAEAKKLNDAQAPK

SEQ ID NO: 19 - protein, 6 aa, Artificial Sequence
  Description of Artificial Sequence Synthetic peptide
ECGPLL

SEQ ID NO: 20 - protein, 6 aa, Artificial Sequence
  Description of Artificial Sequence Synthetic peptide
  MOD_RES (2)..(2): Cys(Ar)
ECGPLL

SEQ ID NO: 21 - protein, 5 aa, Artificial Sequence
  Description of Artificial Sequence Synthetic peptide
GGCGG

(19) United States
(12) Patent Application Publication
(10) Pub. No.: US 2019/0055280 A1
(43) Pub. Date: Feb. 21, 2019

(54) TRANSITION METAL-BASED SELECTIVE FUNCTIONALIZATION OF CHALCOGENS IN BIOMOLECULES

(71) Applicant: Massachusetts Institute of Technoloy, Cambridge, MA (US)

(72) Inventors: Stephen L. Buchwald, Newton, MA (US); Brandley L. Pentelute, Cambridge, MA (US); Alexander M. Spoloyny, Los Angeles, CA (US); Ekaterina V. Vinogradova, Cambridge, MA (US); Chi Zhang, Cambridge, MA (US)

(21) Appl. No.: 15/326,306

(22) PCT Filed: Jul. 15, 2015
(86) PCT No.: PCT/US15/40495

§ 371 Date: Jan. 13, 2017

Related U.S. Application Data
(60) Provisional application No. 62/091,720, filed on Dec. 15, 2014.
(60) Provisional application No. 62/024,769, filed on Jul. 15, 2014.

(51) Int. Cl. C07K 1/107; C07K 1/13; A61K 47/68

(52) U.S. Cl. CPC C07K 1/1077; A61K 47/6803; C07K 1/13

(57) ABSTRACT

Disclosed are methods of selective cysteine and selenocysteine modification on peptide/protein molecules under physiologically relevant conditions. The methods feature several advantages over existing methods of peptide modification, such as specifically toward thiols and selenols over other nucleophiles (e.g., amines, hydroxyls), excellent functional group tolerance, and mild reaction conditions.

### RELATED APPLICATIONS

This application is the U.S. national phase of International Patent Application No. PCT/US2015/040495, filed Jul. 15, 2015, which claims the benefit of priority to U.S. Patent Application Ser. Nos. 62/024,769, filed Jul. 15, 2014; and 62/091,720, filed Dec. 15, 2014, the contents of which are hereby incorporated by reference in their entities.

### GOVERNMENT SUPPORT

This invention was made with Government support under Grant Nos. GM046059 and GM101762 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Sep. 3, 2018, is named MTV-145_01_Sequence_listing.txt and is 10,384 bytes in size.

### BACKGROUND

Post-translational modifications greatly expand the function of proteins. The diversity of potentially reactive functional groups present in biomolecules (e.g., amides, acids, alcohols, amines) combined with the requirement for fast kinetics and mild reaction conditions (e.g., aqueous solvent, pH 6-8, T<37° C.) sets a high bar for the development of new techniques to functionalize proteins. While certain methods have emerged for bioconjugation of natural and unnatural amino acids in protein molecules, functionalization of cysteine residues has remained a challenge. Cysteine is a key residue for the chemical modification of proteins owing to (1) the unique reactivity of the thiol functional group and (2) the low abundance of cysteine residues in naturally occurring proteins.

Cysteine functionalization, and more generally, thiol modification, is an important tool in the chemical, biological, medical, and material sciences. As the only thiol-containing amino acid, cysteine is typically exploited for protein modification using thiol-based reactions. There currently exist several chemical modification techniques allowing for cysteine functionalization in biomolecules. One chemical functionalization, arylation, enables formation of robust arylthioether conjugates with superior stability properties. However, current state of the art arylation methods suffer from several disadvantages. These arylation methods rely on S_{N}Ar chemistry and are fundamentally limited to electron-deficient aromatic reagents, such as, for example, perfluorinated arylation agents. Further, these reagents generate complex mixtures of products, reacting non-specifically with nitrogen-based nucleophiles widely present in biomolecules. Worse still, these current methods exhibit slow reaction rates and require harsh pH and/or solvent conditions. Therefore, there exists a need to develop methods of cysteine functionalization, particularly methods that can tolerate various functional groups, reaction conditions, and that can generate stable products.

### SUMMARY

In certain embodiments, the invention provides a method of functionalizing a thiol or selenol, wherein said method is represented by Scheme 1:

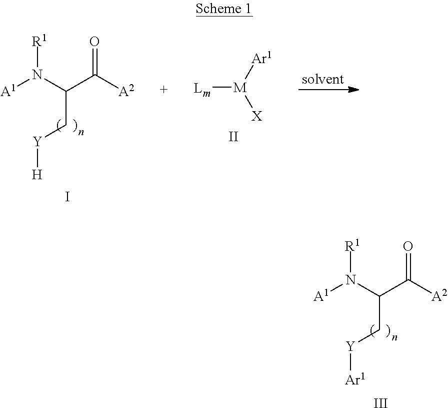

wherein:

A¹ is H, an amine protecting group, alkyl, arylalkyl, acyl, aryl, alkoxycarbonyl, aryloxycarbonyl, a natural or unnatural amino acid, a plurality of natural amino acids or unnatural amino acids, a peptide, an oligopeptide, a polypeptide, a protein, an antibody, or an antibody fragment;

A² is NH₂, NH(amide protecting group), N(amide protecting group), OH, O(carboxylate protecting group), a natural or unnatural amino acid, a plurality of natural amino acids or unnatural amino acids, a peptide, an oligopeptide, a polypeptide, a protein, an antibody, or an antibody fragment;

Y is S or Se;

R¹ is H, alkyl, arylalkyl, acyl, aryl, alkoxycarbonyl, aryloxycarbonyl, a natural or unnatural amino acid, a plurality of natural amino acids or unnatural amino acids, a peptide, an oligopeptide, a polypeptide, a protein, an antibody, or an antibody fragment;

M is Ni, Pd, Pt, Cu, or Au;

Ar¹ is optionally substituted aryl, heteroaryl, alkenyl, or cycloalkenyl;

X is a halide, triflate, tetrafluoroborate, tetraarylborate, hexafluoroantimonate, bis(alkylsulfonyl)amide, tetrafluorophosphate, hexafluorophosphate, alkylsulfonate, haloalkylsulfonate, arylsulfonate, perchlorate, bis(fluoroalkylsulfonyl)amide, bis(arylsulfonyl)amide, (fluoroalkylsulfonyl)(fluoroalkyl-carbonyl)amide, nitrate, nitrite, sulfate, hydrogensulfate, alkyl sulfate, aryl sulfate, carbonate, bicarbonate, carboxylate, phosphate, hydrogen phosphate, dihydrogen phosphate, phosphinate, or hypochlorite;

L is independently for each occurrence a trialkylphosphine, a triarylphosphine, a dialkylarylphosphine, an alkyldiarylphosphine, an (alkenyl)(alkyl)(aryl)phosphine, an alkenyldiarylphosphine, an alkenyldialkylphosphine, a phosphine oxide, a bis(phosphine), a phosphoramide, a triarylphosphonate, an N-heterocyclic carbene, an optionally substituted phenanthroline, an optionally substituted iminopyridine, an optionally substituted 2,2′-bipyridine, an optionally substituted diimine, an optionally substituted triazolylpyridine, or an optionally substituted pyrazolyl pyridine;

n is an integer from 1-5;

m is 1 or 2; and

solvent is a polar protic solvent, a polar aprotic solvent, or a non-polar solvent.

In certain embodiments, the invention relates to a method, wherein said method is represented by Scheme 4:

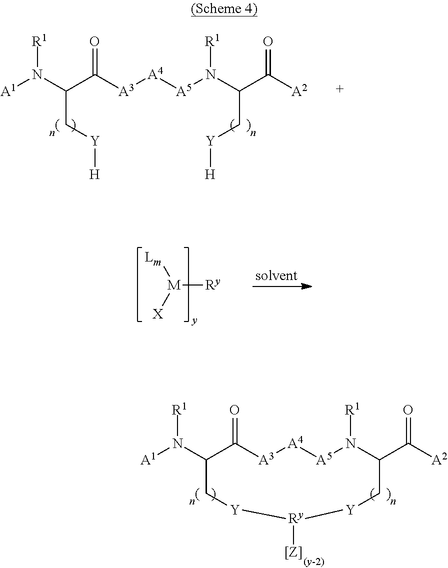

wherein, independently for each occurrence:

A¹ is H, an amine protecting group, alkyl, arylalkyl, acyl, aryl, alkoxycarbonyl, aryloxycarbonyl, a natural or unnatural amino acid, a plurality of natural amino acids or unnatural amino acids, a peptide, an oligopeptide, a polypeptide, a protein, an antibody, or an antibody fragment;

A² is NH₂, NH(amide protecting group), N(amide protecting group), OH, O(carboxylate protecting group), a natural or unnatural amino acid, a plurality of natural amino acids or unnatural amino acids, a peptide, an oligopeptide, a polypeptide, a protein, an antibody, or an antibody fragment;

A³, A⁴, and A⁵ are selected from the group consisting of a natural amino acid, an unnatural amino acid, and a plurality of natural amino acids or unnatural amino acids;

Y is S or Se;

R¹ is H, alkyl, arylalkyl, acyl, aryl, alkoxycarbonyl, aryloxycarbonyl, a natural or unnatural amino acid, a plurality of natural amino acids or unnatural amino acids, a peptide, an oligopeptide, a polypeptide, a protein, an antibody, or an antibody fragment;

M is Ni, Pd, Pt, Cu, or Au;

R^{y} is an optionally substituted bridging moiety, comprising an aromatic group, a heteroaromatic group, an alkene group, or a cycloalkene group;

y is 2, 3, 4, 5, or 6;

X is a halide, triflate, tetrafluoroborate, tetraarylborate, hexafluoroantimonate, bis(alkylsulfonyl)amide, tetrafluorophosphate, hexafluorophosphate, alkylsulfonate, haloalkylsulfonate, arylsulfonate, perchlorate, bis(fluoroalkylsulfonyl)amide, bis(arylsulfonyl)amide, (fluoroalkylsulfonyl)(fluoroalkyl-carbonyl)amide, nitrate, nitrite, sulfate, hydrogensulfate, alkyl sulfate, aryl sulfate, carbonate, bicarbonate, carboxylate, phosphate, hydrogen phosphate, dihydrogen phosphate, phosphinate, or hypochlorite;

L is independently for each occurrence a trialkylphosphine, a triarylphosphine, a dialkylarylphosphine, an alkyldiarylphosphine, an (alkenyl)(alkyl)(aryl)phosphine, an alkenyldiarylphosphine, an alkenyldialkylphosphine, a phosphine oxide, a bis(phosphine), a phosphoramide, a triarylphosphonate, an N-heterocyclic carbene, an optionally substituted phenanthroline, an optionally substituted iminopyridine, an optionally substituted 2,2′-bipyridine, an optionally substituted diimine, an optionally substituted triazolylpyridine, or an optionally substituted pyrazolyl pyridine;

n is an integer from 1-5;

m is 1 or 2;

each Z is independently

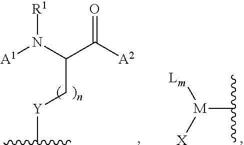

—S-alkyl, —SH, —S—(CH₂)ₙ—CO₂H, —SCH(CH₃)—CO₂H, or —SCH(CO₂H)—CH₂CO₂H; and

solvent is a polar protic solvent, a polar aprotic solvent, or a non-polar solvent.

The invention also provides methods according to Scheme 5:

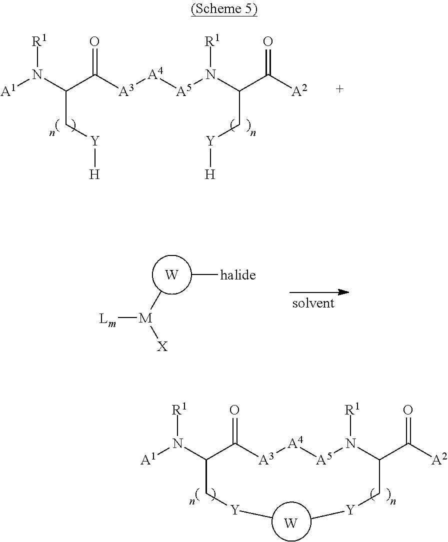

wherein, independently for each occurrence:

A¹ is H, an amine protecting group, alkyl, arylalkyl, acyl, aryl, alkoxycarbonyl, aryloxycarbonyl, a natural or unnatural amino acid, a plurality of natural amino acids or unnatural amino acids, a peptide, an oligopeptide, a polypeptide, a protein, an antibody, or an antibody fragment;

A² is NH₂, NH(amide protecting group), N(amide protecting group), OH, O(carboxylate protecting group), a natural or unnatural amino acid, a plurality of natural amino acids or unnatural amino acids, a peptide, an oligopeptide, a polypeptide, a protein, an antibody, or an antibody fragment;

A³, A⁴, and A⁵ are selected from the group consisting of a natural amino acid, an unnatural amino acid, and a plurality of natural amino acids or unnatural amino acids;

Y is S or Se;

R¹ is H, alkyl, arylalkyl, acyl, aryl, alkoxycarbonyl, aryloxycarbonyl, a natural or unnatural amino acid, a plurality of natural amino acids or unnatural amino acids, a peptide, an oligopeptide, a polypeptide, a protein, an antibody, or an antibody fragment;

M is Ni, Pd, Pt, Cu, or Au;

X is a halide, triflate, tetrafluoroborate, tetraarylborate, hexafluoroantimonate, bis(alkylsulfonyl)amide, tetrafluorophosphate, hexafluorophosphate, alkylsulfonate, haloalkylsulfonate, arylsulfonate, perchlorate, bis(fluoroalkylsulfonyl)amide, bis(arylsulfonyl)amide, (fluoroalkylsulfonyl)(fluoroalkyl-carbonyl)amide, nitrate, nitrite, sulfate, hydrogensulfate, alkyl sulfate, aryl sulfate, carbonate, bicarbonate, carboxylate, phosphate, hydrogen phosphate, dihydrogen phosphate, phosphinate, or hypochlorite;

L is independently for each occurrence a trialkylphosphine, a triarylphosphine, a dialkylarylphosphine, an alkyldiarylphosphine, an (alkenyl)(alkyl)(aryl)phosphine, an alkenyldiarylphosphine, an alkenyldialkylphosphine, a phosphine oxide, a bis(phosphine), a phosphoramide, a triarylphosphonate, an N-heterocyclic carbene, an optionally substituted phenanthroline, an optionally substituted iminopyridine, an optionally substituted 2,2′-bipyridine, an optionally substituted diimine, an optionally substituted triazolylpyridine, or an optionally substituted pyrazolyl pyridine;

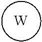

is aryl, heteroaryl, alkenyl, or cycloalkenyl, wherein

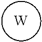

is optionally further substituted by one or more substituents selected from halide, acyl, azide, isothiocyanate, alkyl, aralkyl, alkenyl, alkynyl or protected alkynyl, alkoxyl, arylcarbonyl, cycloalkyl, formyl, haloalkyl, hydroxyl, amino, nitro, sulfhydryl, amido, phosphonate, phosphinate, alkylthio, sulfonyl, sulfonamido, heterocyclyl, aryl, heteroaryl, —CF₃, —CF₂R⁷, —CFR⁷₂, —CN, polyethylene glycol, polyethylene imine, —(CH₂)ₚ-FG-R⁷, and Z;

Z is

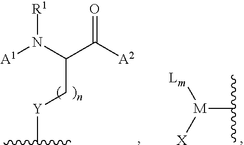

—S-alkyl, —SH, —S—(CH₂)ₙ—CO₂H, —SCH(CH₃)—CO₂H, or —SCH(CO₂H)—CH₂CO₂H;

p is independently for each occurrence an integer from 0-10;

FG is independently for each occurrence selected from the group consisting of C(O), CO₂, O(CO), C(O)NR⁷, NR⁷C(O), O, Si(R⁷)₂, C(NR⁷), (R⁷)₂N(CO)N(R⁷)₂, OC(O)NR⁷, NR⁷C(O)O, and C(N═N);

R⁷ is independently for each occurrence selected from the group consisting of H, alkyl, cycloalkyl, aryl, aralkyl, alkenyl, and alkynyl;

n is an integer from 1-5;

m is 1 or 2; and

solvent is a polar protic solvent, a polar aprotic solvent, or a non-polar solvent.

In certain embodiments, L is selected from the group consisting of PPh₃, Ph₂P—CH₃, PhP(CH₃)₂, P(o-tol)₃, PCy₃, P(tBu)₃, BINAP, dppb, dppe, dppf, dppp,

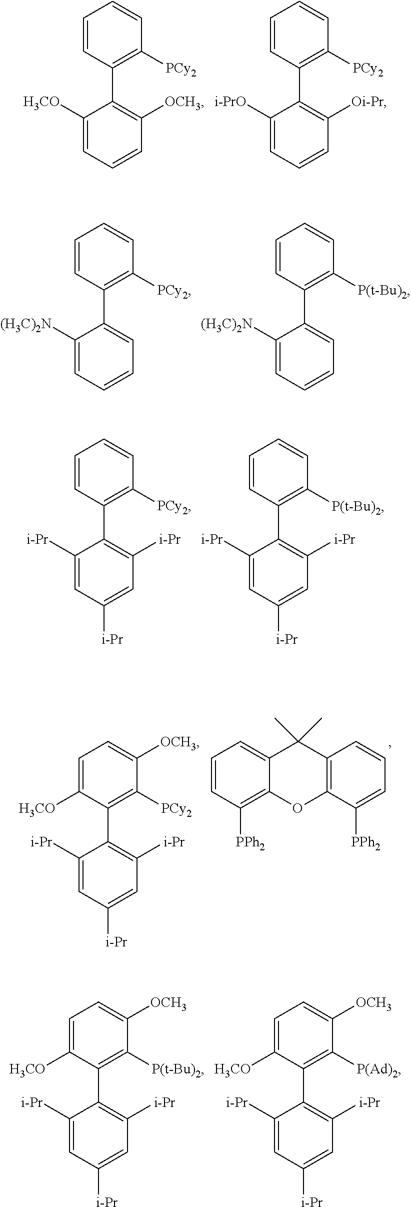

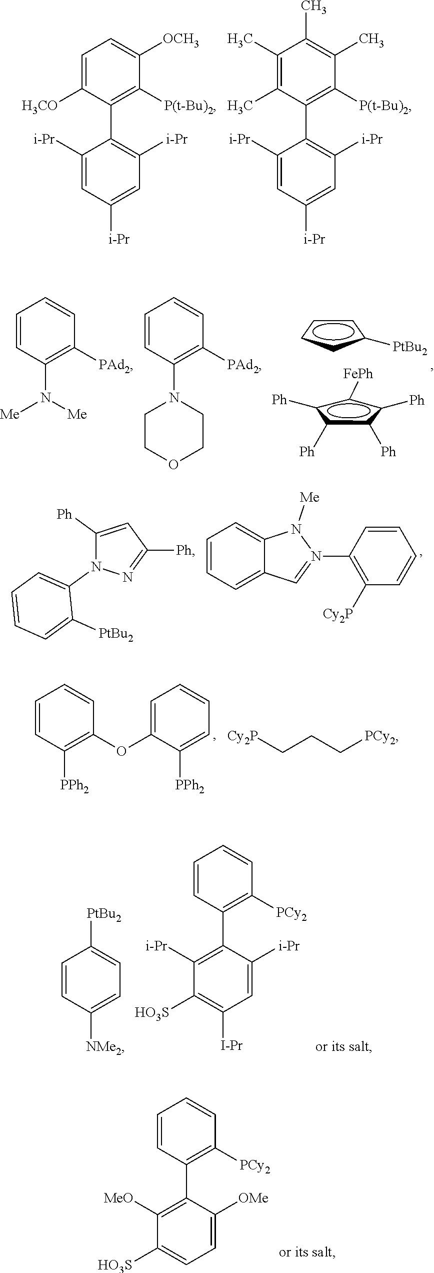

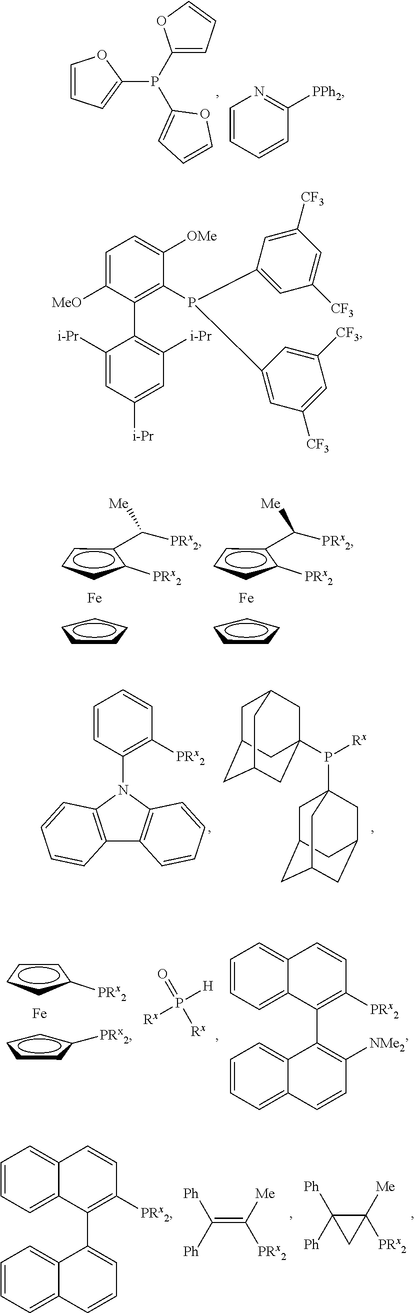

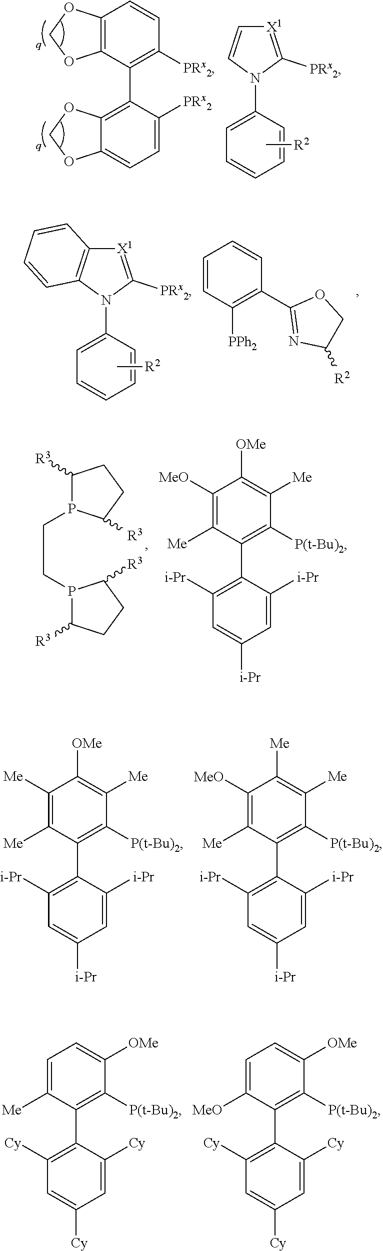

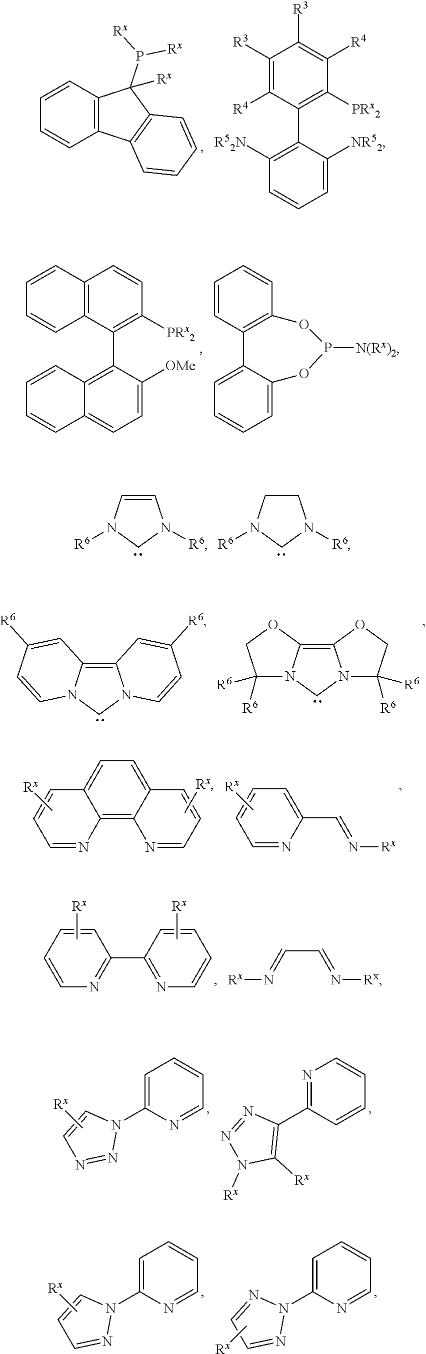

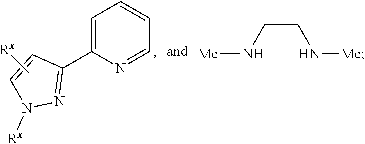

R^{x} is independently for each occurrence alkyl, aralkyl, cycloalkyl, or aryl;

X¹ is CH or N;

R² is H or alkyl;

R³ is H or alkyl;

R⁴ is H, alkoxy, or alkyl;

R⁵ is alkyl or aryl;

R⁶ is alkyl or aryl; and

q is 1, 2, 3, or 4.

In certain embodiments, M is Ni or Pd.

In certain embodiments, X is triflate or halide.

In certain embodiments, Ar¹ is (C₆-C₁₀)carbocyclic aryl, (C₃-C₁₂)heteroaryl, (C₃-C₁₄)polycyclic aryl, or alkenyl; and Ar¹ is optionally substituted by one or more substituents independently selected from the group consisting of halide, acyl, azide, isothiocyanate, alkyl, aralkyl, alkenyl, alkynyl or protected alkynyl, alkoxyl, arylcarbonyl, cycloalkyl, formyl, haloalkyl, hydroxyl, amino, nitro, sulfhydryl, amido, phosphonate, phosphinate, alkylthio, sulfonyl, sulfonamido, heterocyclyl, aryl, heteroaryl, —CF₃, —CF₂R⁷, —CFR⁷₂, —CN, polyethylene glycol, polyethylene imine, and —(CH₂)ₚ-FG-R⁷;

p is independently for each occurrence an integer from 0-10;

FG is independently for each occurrence selected from the group consisting of C(O), CO₂, O(CO), C(O)NR⁷, NR⁷C(O), O, Si(R⁷)₂, C(NR⁷), (R⁷)₂N(CO)N(R⁷)₂, OC(O)NR⁷, NR⁷C(O)O, and C(N═N);

R⁷ is independently for each occurrence selected from the group consisting of H, alkyl, cycloalkyl, aryl, aralkyl, alkenyl, and alkynyl; and

if two or more substituents are present on Ar¹, then two of said substituents taken together may form a ring.

In certain embodiments, Ar¹ is covalently linked to a fluorophore, an imaging agent, a detection agent, a biomolecule, a therapeutic agent, a lipophilic moiety, a member of a high-affinity binding pair, or a cell-receptor targeting agent. In one embodiment, Ar¹ is linked to biotin. In another embodiment, Ar¹ is linked to fluorescein. In one embodiment, the therapeutic agent is trametinib, topotecan, abiraterone, dabrafenib, or vandetanib.

In certain embodiments, Ar¹ is comprised by a fluorophore.

In certain embodiments, Ar¹ is comprised by a therapeutic agent.

In certain embodiments, A¹ and A² are independently a natural or unnatural amino acid, a plurality of natural or unnatural amino acids, a peptide, an oligopeptide, a polypeptide, or a protein.

In certain embodiments, A¹ or A² comprises arginine, histidine, lysine, aspartic acid, glutamic acid, serine, threonine, asparagine, glutamine, proline, tyrosine, or tryptophan.

In certain embodiments, the invention is a method of functionalizing a thiol or selenol, wherein the limiting reagent is

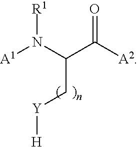

In certain embodiments, when A¹ or A² comprises an —SH or —SeH moiety, the molar ratio of the amount of

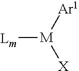

to the amount of

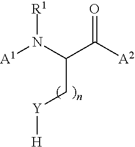

multiplied by the aggregate number of —SH and —SeH moieties in

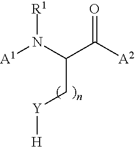

is greater than 1:1.

In certain embodiments of the method of the invention, A¹ and A² are covalently linked.

In certain embodiments, the solvent used in the methods of the invention comprises water.

In certain embodiments, the solvent used in the methods of the invention comprises an aqueous buffer.

In other embodiments, the invention relates to a method of functionalizing a thiol or selenol in a biopolymer, comprising contacting a biopolymer comprising a thiol or selenol moiety with a reagent of structural formula II, thereby generating a functionalized biopolymer, wherein the thiol or selenol moiety has been transformed to —S—Ar¹ or —Se—Ar¹.

In certain embodiments, the biopolymer is an oligonucleotide, a polynucleotide, an oligosaccharide, or a polysaccharide.

### BRIEF DESCRIPTION OF THE FIGURES

FIG. 1 depicts exemplary ligands (e.g., tBuBrettPhos=L15; AdBrettPhos=L16; and RockPhos=L17) useful in the invention.

FIG. 2 depicts exemplary ligands useful in the invention.

FIG. 3 depicts a representative synthesis of a Pd-based reagent for cysteine and selenocysteine arylation.

FIG. 4(a) depicts selective cysteine S-arylation in a unprotected model peptide.

FIG. 4(b) depicts an LCMS trace of the product of S-arylation of the unprotected model peptide.

FIG. 5 is an LCMS trace for AKLTGF-NH(CH₂C₆F₅) under arylation conditions, demonstrating no arylation (e.g., at threonine or lysine).

FIG. 6 is LCMS traces of products from arylation of Cys-containing peptides in aqueous media.

FIGS. 7(a)-7(f) depict LCMS traces of S-arylated products prepared from peptide 6 using the corresponding Pd(II) reagents.

FIG. 8(a) depicts exemplary species of S-arylated forms of peptide 6 obtained using the corresponding Pd(II) reagents.

FIG. 8(b) depicts exemplary pharmaceutical agents suitable for bioconjugation to the peptide.

FIG. 9 shows a representative arylation of DARPin using a fluorescein-containing Pd(II) reagent (left), and SDS-PAGE analysis of the labeling (right).

FIG. 10 depicts exemplary strategies for arylation of Cys sidechains in antibodies using Pd-based reagents.

FIG. 11 depicts an experimental scheme for and results from fluorescein arylation of human IgG1 antibody.

FIG. 12(a) depicts an exemplary synthesis of a polymetalated reagent (bifunctional) for the formation of a cyclic or stapled peptide.

FIG. 12(b) depicts an exemplary synthesis of a polymetalated reagent (trifunctional) for the formation of a cyclic, polycyclic, or stapled peptide.

FIG. 13 depicts a schematic of a representative procedure for antibody-drug conjugation of Trastuzumab with Vandetanib (represented by stars) using a method of the invention.

FIG. 14 is a graph showing the stability of P2 cysteine conjugates under oxidative conditions.

FIG. 15 has four panels (top, a, b, and c) depicting protein modification using palladium reagents of the invention. The reaction scheme is shown in the top panel. Panels a, b, and c show quantitative modification of cysteine residues at a) the N-terminus (P4), b) a loop (P5), and c) the C-terminus (P6) of proteins with coumarin after the reaction with palladium complex 1D.

FIG. 16 has four panels (top, a, b, and c) depicting control reactions for protein labeling with palladium complex 1D. The reaction scheme is shown in the top panel. Panels a, b, and c show that the resulting proteins P7-P9 do not contain cysteine residues.

FIG. 17 has four panels (top, a, b, and c) depicting protein modification using palladium complex 1J. The reaction scheme is shown in the top panel. Panels a, b, and c show quantitative modification of cysteine residues at a) the N-terminus (P4), b) a loop (P5), and c) the C-terminus (P6) of proteins with a drug molecule after the reaction with palladium complex 1J.

FIG. 18 has four panels (top, a, b, and c) depicting control reactions for protein labeling with palladium complex 1J. The reaction scheme is shown in the top panel. Panels a, b, and c show that the resulting proteins P7-P9 do not contain cysteine residues.

FIG. 19 has three panels (top, middle, and bottom) depicting a reaction scheme (top) of a double cross coupling reaction, and traces showing the various products in 1:1 CH₃CN:H₂O (middle) and 5:95 CH₃CN:H₂O (bottom).

FIG. 20 depicts a schematic of a representative procedure for synthesis of a stapled peptide using a Pd-based haloarylation reagent.

FIG. 21 depicts schematic of a representative procedure for arylation of Cys residues using an air-stable Ph-mesylate palladium precatalyst and aryl halide.

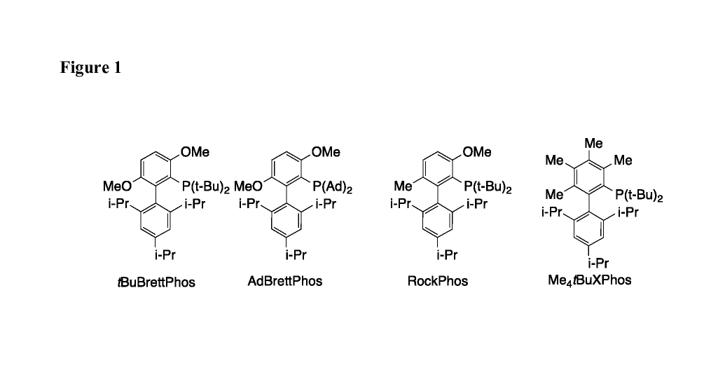

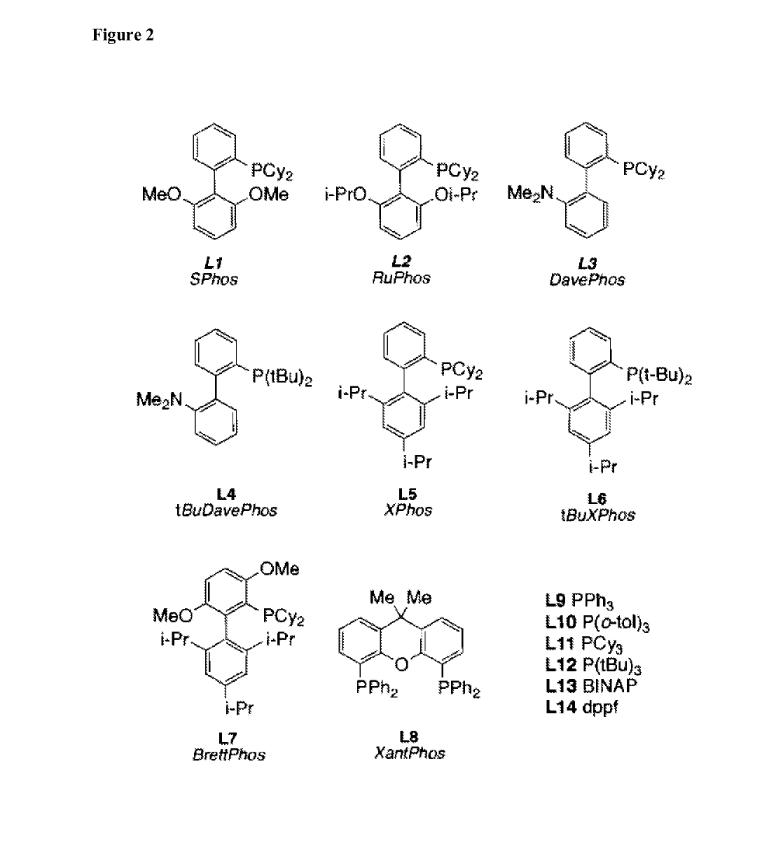

### DETAILED DESCRIPTION

### Overview

In certain embodiments, the invention relates to a method of functionalizing a thiol or selenol, wherein the method is represented by Scheme 1:

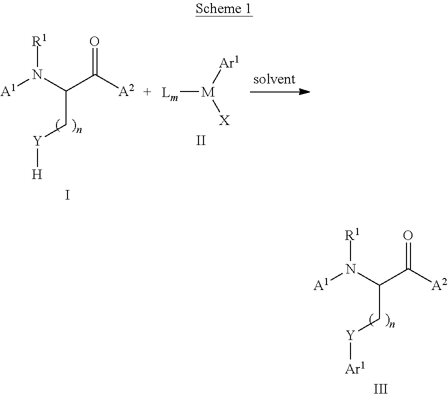

wherein:

A¹ is H, an amine protecting group, alkyl, arylalkyl, acyl, aryl, alkoxycarbonyl, aryloxycarbonyl, a natural or unnatural amino acid, a plurality of natural amino acids or unnatural amino acids, a peptide, an oligopeptide, a polypeptide, a protein, an antibody, or an antibody fragment;

A² is NH₂, NH(amide protecting group), N(amide protecting group), OH, O(carboxylate protecting group), a natural or unnatural amino acid, a plurality of natural amino acids or unnatural amino acids, a peptide, an oligopeptide, a polypeptide, a protein, an antibody, or an antibody fragment;

Y is S or Se;

R¹ is H, alkyl, arylalkyl, acyl, aryl, alkoxycarbonyl, aryloxycarbonyl, a natural or unnatural amino acid, a plurality of natural amino acids or unnatural amino acids, a peptide, an oligopeptide, a polypeptide, a protein, an antibody, or an antibody fragment;

M is Ni, Pd, Pt, Cu, or Au;

Ar¹ is optionally substituted aryl, heteroaryl, alkenyl, or cycloalkenyl;

X is a halide, triflate, tetrafluoroborate, tetraarylborate, hexafluoroantimonate, bis(alkylsulfonyl)amide, tetrafluorophosphate, hexafluorophosphate, alkylsulfonate, haloalkylsulfonate, arylsulfonate, perchlorate, bis(fluoroalkylsulfonyl)amide, bis(arylsulfonyl)amide, (fluoroalkylsulfonyl)(fluoroalkyl-carbonyl)amide, nitrate, nitrite, sulfate, hydrogensulfate, alkyl sulfate, aryl sulfate, carbonate, bicarbonate, carboxylate, phosphate, hydrogen phosphate, dihydrogen phosphate, phosphinate, or hypochlorite; L is independently for each occurrence a trialkylphosphine, a triarylphosphine, a dialkylarylphosphine, an alkyldiarylphosphine, an (alkenyl)(alkyl)(aryl)phosphine, an alkenyldiarylphosphine, an alkenyldialkylphosphine, a phosphine oxide, a bis(phosphine), a phosphoramide, a triarylphosphonate, an N-heterocyclic carbene, an optionally substituted phenanthroline, an optionally substituted iminopyridine, an optionally substituted 2,2′-bipyridine, an optionally substituted diimine, an optionally substituted triazolylpyridine, or an optionally substituted pyrazolyl pyridine;

n is an integer from 1-5;

m is 1 or 2; and

solvent is a polar protic solvent, a polar aprotic solvent, or a non-polar solvent.

This method features several significant advantages over existing functionalization methods, such as specificity for functionalization of thiols and selenols over other reactive functional groups (e.g., hydroxyls, amines), excellent functional group tolerance, and mild reaction conditions in both polar organic and buffered aqueous solvent media. Furthermore, kinetic studies demonstrate that the methods of the invention are fast, resulting in complete labeling at micromolar concentrations of biomolecules within minutes. The methods presented herein are widely applicable for modifications of biomolecules containing amino acids bearing thiol or selenol moieties. The ability to selectively chemically modify biomolecules is an important application relevant to research and development in the pharmaceutical and biotechnology industries.

In certain embodiments, the invention relates to selective cysteine and selenocysteine modification on unprotected peptide/protein molecules under physiologically relevant conditions. This process exhibits specificity towards cysteine (Cys) and selenocysteine (Sec) over other competing nucleophilic amino acids (e.g., serine, threonine, lysine), excellent functional group tolerance, and mild reaction conditions.

In certain embodiments, the invention is a method according to Scheme 1, wherein m is an integer from 0-3.

In certain embodiments, the thiol or selenol that is functionalized in the methods of the invention is an alpha amino acid having the structure of formula (I):

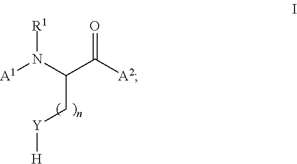

wherein A¹, A², Y, n, and R¹ are defined as above. In certain embodiments, the thiol is cysteine and the selenol is selenocysteine. In certain embodiments, n is 1 or 2.

### Exemplary Functionalization Complexes

In certain embodiments, the invention relates to a method of functionalizing (e.g., arylating) a thiol or selenol according to Scheme 1, wherein the functionalization agent is a compound of formula (II):

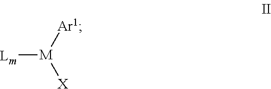

wherein L is a ligand, X is a halide or a triflate, m is 1 or 2, and Ar¹ is optionally substituted aryl, heteroaryl, alkenyl, or cycloalkenyl.

In certain embodiments, the invention relates to a method according to Scheme 1, wherein the functionalization agent is a compound of formula (II), wherein m is an integer from 0-3. In certain embodiments, m is an integer from 1-3. In certain embodiments, m is 1 or 2. In more particular embodiments, m is 1. In certain embodiments in which m is 2 or 3, one instance of L is covalently connected via a linker moiety to one or more other instances of L. In such certain embodiments, M, taken together with two or three instances of ligand, is a cyclic or bicyclic structure.

In certain embodiments, the ligand L of formula (II) is a ligand described in U.S. Pat. No. 7,858,784, which is hereby incorporated by reference in its entirety.

In certain embodiments, the ligand L of formula (II) is a ligand described in U.S. Patent Application Publication No. 2011/0015401, which is hereby incorporated by reference in its entirety.

In certain embodiments, the ligand L of formula (II) is a trialkylphosphine, a triarylphosphine, a dialkylarylphosphine, an alkyldiarylphosphine, an (alkenyl)(alkyl)(aryl)phosphine, an alkenyldiarylphosphine, an alkenyldialkylphosphine, a phosphine oxide, a bis(phosphine), a phosphoramide, a triarylphosphonate, an N-heterocyclic carbene, an optionally substituted phenanthroline, an optionally substituted iminopyridine, an optionally substituted 2,2′-bipyridine, an optionally substituted diimine, an optionally substituted triazolylpyridine, or an optionally substituted pyrazolyl pyridine. In certain embodiments, the ligand L of formula (II) is a trialkylphosphine, a triarylphosphine, a dialkylarylphosphine, an alkyldiarylphosphine, an (alkenyl)(alkyl)(aryl)phosphine, an alkenyldiarylphosphine, an alkenyldialkylphosphine, a phosphine oxide, a bis(phosphine), a phosphoramide, or a triarylphosphonate.

In certain embodiments, the ligand L of formula (II) is selected from the group consisting of PPh₃, Ph₂P—CH₃, PhP(CH₃)₂, P(o-tol)₃, PCy₃, P(tBu)₃, BINAP, dppb, dppe,

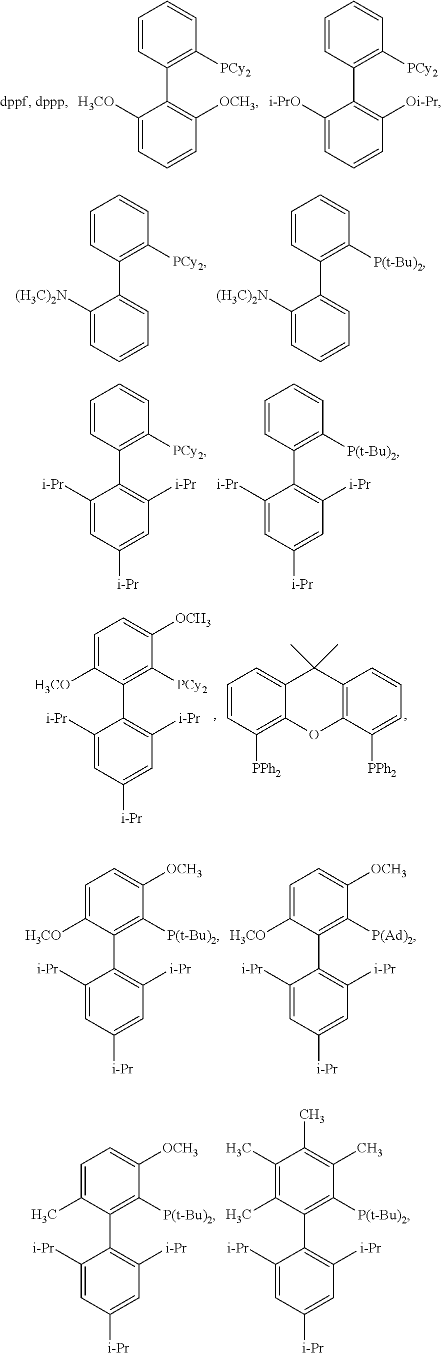

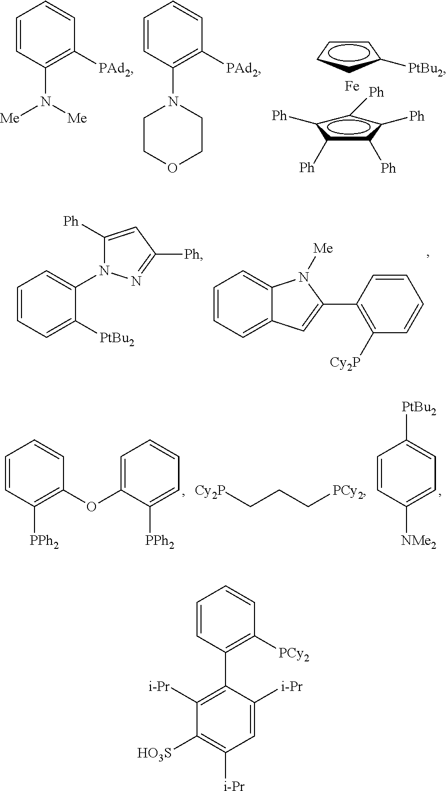

or its salt,

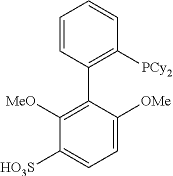

or its salt,

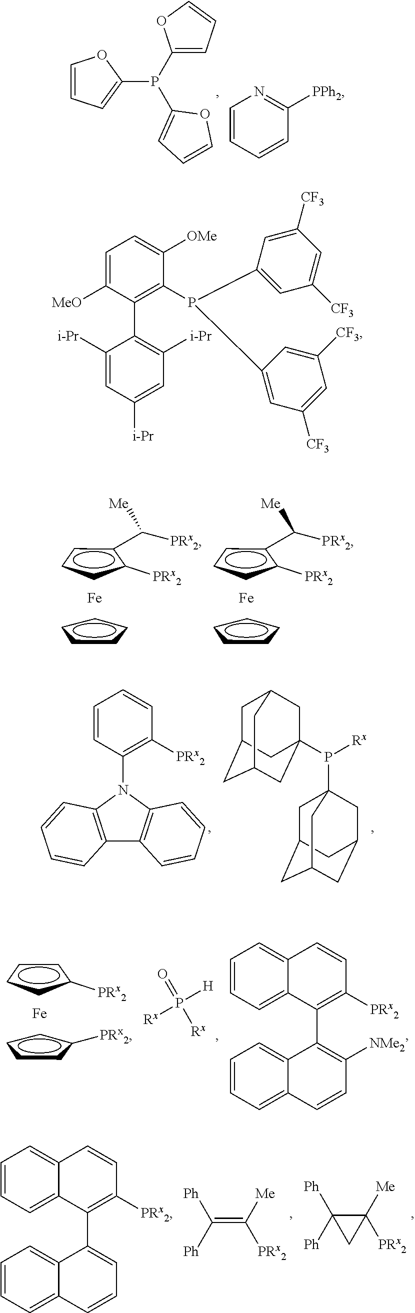

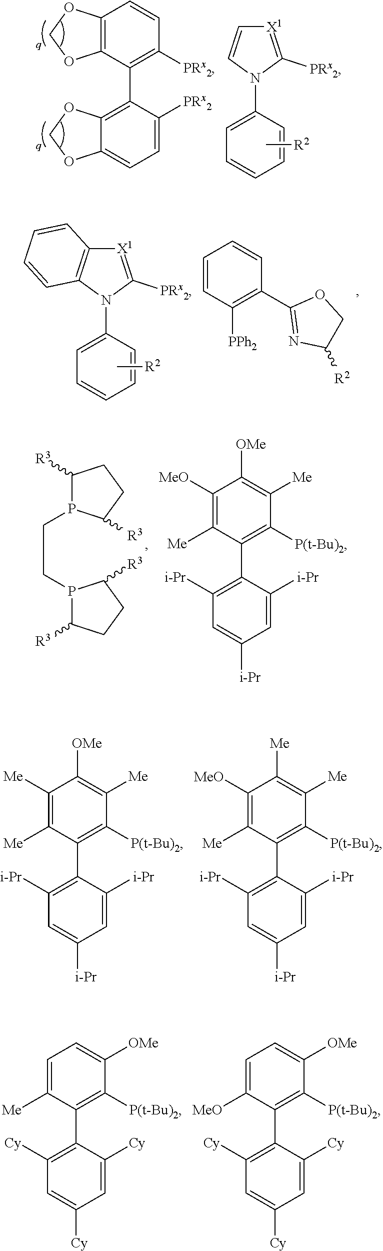

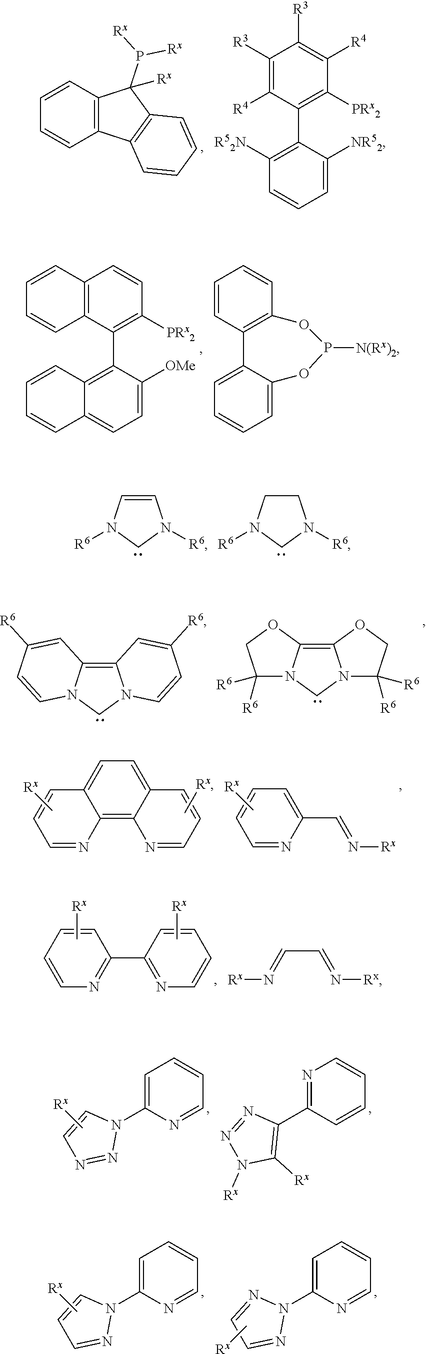

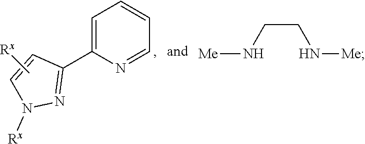

R^{x} is alkyl, aralkyl, cycloalkyl, or aryl;

X¹ is CH or N;

R² is H or alkyl;

R³ is H or alkyl;

R⁴ is H, alkoxy, or alkyl;

R⁵ is alkyl or aryl;

R⁶ is alkyl or aryl; and

q is 1, 2, 3, or 4.

In certain embodiments, X of formula (II) is X is a halide (e.g., fluoride, chloride, bromide, iodide) or a triflate.

In certain embodiments, X of formula (II) is selected from the group consisting of boron tetrafluoride, tetraarylborates (such as B(C₆F₅)₄⁻ and (B[3,5-(CF₃)₂C₆H₃]₄)⁻), hexafluoroantimonate, phosphorus tetrafluoride, phosphorus hexafluoride, alkylsulfonate, haloalkylsulfonate, arylsulfonate, perchlorate, bis(alkylsulfonyl)amide, halide, bis(fluoroalkylsulfonyl)amide, bis(arylsulfonyl)amide, (fluoroalkylsulfonyl)(fluoroalkyl-carbonyl)amide, nitrate, nitrite, sulfate, hydrogensulfate, alkyl sulfate, aryl sulfate, carbonate, bicarbonate, carboxylate, phosphate, hydrogen phosphate, dihydrogen phosphate, phosphinate, and hypochlorite.

In certain embodiments, X of formula (II) is alkylsulfonate; and the alkyl is substituted alkyl. In certain embodiments, X of formula (II) is alkylsulfonate; and the alkyl is unsubstituted alkyl.

In certain embodiments, X of formula (II) is alkylsulfonate; and the alkyl is methyl, ethyl, propyl, or butyl. In certain embodiments, X of formula (II) is alkylsulfonate; and the alkyl is methyl or ethyl.

In certain embodiments, X of formula (II) is haloalkylsulfonate. In certain embodiments, X of formula (II) is fluoroalkylsulfonate.

In certain embodiments, X of formula (II) is fluoromethylsulfonate. In certain embodiments, X is trifluoromethylsulfonate.

In certain embodiments, X of formula (II) is cycloalkylalkylsulfonate. In certain embodiments, X is

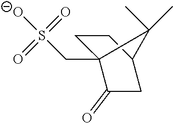

or its enantiomer.

In certain embodiments, m of formula (II) is 1 or 2. In certain embodiments, m is 1.

In certain embodiments, Ar¹ of formula (II) is optionally substituted aryl, heteroaryl, alkenyl, or cycloalkenyl. In certain embodiments, Ar¹ is optionally substituted aryl or heteroaryl group.

In certain embodiments, Ar¹ of formula (II) is (C₆-C₁₀)carbocyclic aryl, (C₃-C₁₂)heteroaryl, (C₃-C₁₄)polycyclic aryl, or alkenyl; and Ar¹ is optionally substituted by one or more substituents independently selected from the group consisting of halide, acyl, azide, isothiocyanate, alkyl, aralkyl, alkenyl, alkynyl or protected alkynyl, alkoxyl, arylcarbonyl, cycloalkyl, formyl, haloalkyl, hydroxyl, amino, nitro, sulfhydryl, amido, phosphonate, phosphinate, alkylthio, sulfonyl, sulfonamido, heterocyclyl, aryl, heteroaryl, —CF₃, —CF₂R⁷, —CFR⁷₂, —CN, polyethylene glycol, polyethylene imide, and —(CH₂)ₙ-FG-R⁷;

n is independently for each occurrence an integer from 0-10;

FG is independently for each occurrence selected from the group consisting of C(O), CO₂, O(CO), C(O)NR⁷, NR⁷C(O), O, Si(R⁷)₂, C(NR⁷), (R⁷)₂N(CO)N(R⁷)₂, OC(O)NR⁷, NR⁷C(O)O, and C(N═N);

R⁷ is independently for each occurrence selected from the group consisting of H, alkyl, cycloalkyl, aryl, aralkyl, alkenyl, and alkynyl; and

if two or more substituents are present on Ar¹, then two of said substituents taken together may form a ring.

In certain embodiments, Ar¹ of formula (II) is covalently linked to a fluorophore, an imaging agent, a detection agent, a biomolecule, a therapeutic agent, a lipophilic moiety, a member of a high-affinity binding pair, or a cell-receptor targeting agent. In certain embodiments, the invention relates to any one of the aforementioned compounds, wherein Ar¹ is covalently linked to biotin. In certain embodiments, the invention relates to any one of the aforementioned compounds, wherein Ar¹ is covalently linked to fluorescein. In certain embodiments, the invention relates to any of the aforementioned compounds, wherein Ar¹ is covalently linked to a therapeutic agent; and the therapeutic agent is trametinib, topotecan, abiraterone, dabrafenib, or vandetanib.

In certain other embodiments, Ar¹ of formula (II) is comprised by a fluorophore. In certain embodiments, the invention relates to any of the aforementioned compounds, wherein Ar¹ is comprised by a therapeutic agent. In certain embodiments, the therapeutic agent is the trametinib, topotecan, abiraterone, dabrafenib, or vandetanib.

In certain embodiments, the fluorophore is a derivative of xanthene, fluorescein, rhodamine, coumarin, naphthalene, anathracene, oxadiazole, pyrene, acridine, tetrapyrrole, arylmethine, boron-dipyrromethene (BODIPY), or a cyanine dye. In certain other embodiments, the fluorophore is a fluorescent protein. In certain embodiments, the detection agent is for example, a nanoparticle, an MRI contrast agent, a dye moiety, or a radionuclide. In certain other embodiments, a biomolecule is a protein, a peptide, a monosaccharide, a disaccharide, an oligosaccharide, a polysaccharide, a lipid, a glycolipid, a glycerolipid, a phospholipid, a hormone, a neurotransmitter, a nucleic acid, a nucleotide, a nucleoside, a sterol, a metabolite, a vitamin, or a natural product.

In certain embodiments, a therapeutic agent is a compound or substructure of a compound that brings about a therapeutic effect in a subject to which the agent is administered. In certain embodiments, the therapeutic agent is toxic to certain cells. Exemplary therapeutic agents that are covalently linked to Ar¹ of formula (II) include trametinib, topotecan, abiraterone, dabrafenib, or vandetanib.

In certain embodiments, the lipophilic moiety enables the compound bearing Ar¹ to have an affinity for, or be soluble in, lipids, fats, oils, ad non-polar solvents, as described herein. Exemplary lipophilic moieties include amphiphilic surfactants, such as cinnamic acid.

In certain embodiments, the cell-receptor targeting agent is a ligand such as an epitope, a peptide, an antibody, a small organic compound, a neurotransmitter. High-affinity binding pairs include biotin-avidin, biotin-streptavidin, ligand-cell receptor, S-Peptide and Ribonuclease A, digoxigenin and its receptor, and complementary oligonucleotide pairs.

### Exemplary Methods

In certain embodiments, the invention relates to a method of Scheme 1:

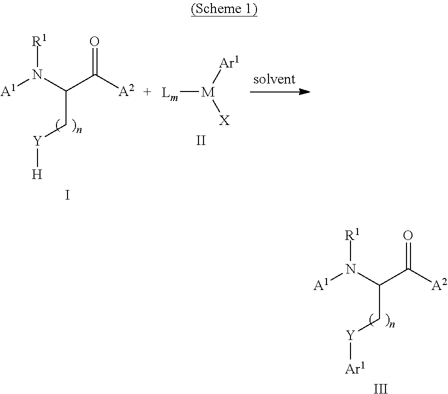

wherein,

A¹ is H, an amine protecting group, alkyl, arylalkyl, acyl, aryl, alkoxycarbonyl, aryloxycarbonyl, a natural or unnatural amino acid, a plurality of natural amino acids or unnatural amino acids, a peptide, an oligopeptide, a polypeptide, a protein, an antibody, or an antibody fragment;

A² is NH₂, NH(amide protecting group), N(amide protecting group), OH, O(carboxylate protecting group), a natural or unnatural amino acid, a plurality of natural amino acids or unnatural amino acids, a peptide, an oligopeptide, a polypeptide, a protein, an antibody, or an antibody fragment;

Y is S or Se;

R¹ is H, alkyl, arylalkyl, acyl, aryl, alkoxycarbonyl, aryloxycarbonyl, a natural or unnatural amino acid, a plurality of natural amino acids or unnatural amino acids, a peptide, an oligopeptide, a polypeptide, a protein, an antibody, or an antibody fragment;

M is Ni, Pd, Pt, Cu, or Au;

Ar¹ is optionally substituted aryl, heteroaryl, alkenyl, or cycloalkenyl;

X is a halide, triflate, tetrafluoroborate, tetraarylborate, hexafluoroantimonate, bis(alkylsulfonyl)amide, tetrafluorophosphate, hexafluorophosphate, alkylsulfonate, haloalkylsulfonate, arylsulfonate, perchlorate, bis(fluoroalkylsulfonyl)amide, bis(arylsulfonyl)amide, (fluoroalkylsulfonyl)(fluoroalkyl-carbonyl)amide, nitrate, nitrite, sulfate, hydrogensulfate, alkyl sulfate, aryl sulfate, carbonate, bicarbonate, carboxylate, phosphate, hydrogen phosphate, dihydrogen phosphate, phosphinate, or hypochlorite;

L is independently for each occurrence a trialkylphosphine, a triarylphosphine, a dialkylarylphosphine, an alkyldiarylphosphine, an (alkenyl)(alkyl)(aryl)phosphine, an alkenyldiarylphosphine, an alkenyldialkylphosphine, a phosphine oxide, a bis(phosphine), a phosphoramide, a triarylphosphonate, an N-heterocyclic carbene, an optionally substituted phenanthroline, an optionally substituted iminopyridine, an optionally substituted 2,2′-bipyridine, an optionally substituted diimine, an optionally substituted triazolylpyridine, or an optionally substituted pyrazolyl pyridine;

n is an integer from 1-5;

m is 1 or 2; and

solvent is a polar protic solvent, a polar aprotic solvent, or a non-polar solvent.

In certain embodiments, the invention relates to a method, wherein said method is represented by Scheme 4:

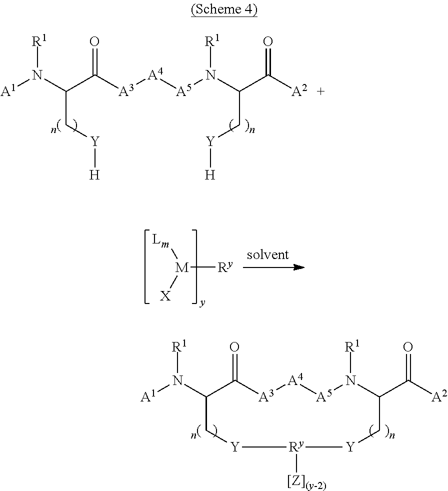

wherein, independently for each occurrence:

A¹ is H, an amine protecting group, alkyl, arylalkyl, acyl, aryl, alkoxycarbonyl, aryloxycarbonyl, a natural or unnatural amino acid, a plurality of natural amino acids or unnatural amino acids, a peptide, an oligopeptide, a polypeptide, a protein, an antibody, or an antibody fragment;

A² is NH₂, NH(amide protecting group), N(amide protecting group), OH, O(carboxylate protecting group), a natural or unnatural amino acid, a plurality of natural amino acids or unnatural amino acids, a peptide, an oligopeptide, a polypeptide, a protein, an antibody, or an antibody fragment;

A³, A⁴, and A⁵ are selected from the group consisting of a natural amino acid, an unnatural amino acid, and a plurality of natural amino acids or unnatural amino acids;

Y is S or Se;

R¹ is H, alkyl, arylalkyl, acyl, aryl, alkoxycarbonyl, aryloxycarbonyl, a natural or unnatural amino acid, a plurality of natural amino acids or unnatural amino acids, a peptide, an oligopeptide, a polypeptide, a protein, an antibody, or an antibody fragment;

M is Ni, Pd, Pt, Cu, or Au;

R^{y} is an optionally substituted bridging moiety, comprising an aromatic group, a heteroaromatic group, an alkene group, or a cycloalkene group;

y is 2, 3, 4, 5, or 6;

X is a halide, triflate, tetrafluoroborate, tetraarylborate, hexafluoroantimonate, bis(alkylsulfonyl)amide, tetrafluorophosphate, hexafluorophosphate, alkylsulfonate, haloalkylsulfonate, arylsulfonate, perchlorate, bis(fluoroalkylsulfonyl)amide, bis(arylsulfonyl)amide, (fluoroalkylsulfonyl)(fluoroalkyl-carbonyl)amide, nitrate, nitrite, sulfate, hydrogensulfate, alkyl sulfate, aryl sulfate, carbonate, bicarbonate, carboxylate, phosphate, hydrogen phosphate, dihydrogen phosphate, phosphinate, or hypochlorite;

L is independently for each occurrence a trialkylphosphine, a triarylphosphine, a dialkylarylphosphine, an alkyldiarylphosphine, an (alkenyl)(alkyl)(aryl)phosphine, an alkenyldiarylphosphine, an alkenyldialkylphosphine, a phosphine oxide, a bis(phosphine), a phosphoramide, a triarylphosphonate, an N-heterocyclic carbene, an optionally substituted phenanthroline, an optionally substituted iminopyridine, an optionally substituted 2,2′-bipyridine, an optionally substituted diimine, an optionally substituted triazolylpyridine, or an optionally substituted pyrazolyl pyridine;

n is an integer from 1-5;

m is 1 or 2;

each Z is independently

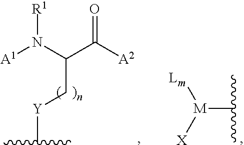

—S-alkyl, —SH, —S—(CH₂)ₙ—CO₂H, —SCH(CH₃)—CO₂H, or —SCH(CO₂H)—CH₂CO₂H; and

solvent is a polar protic solvent, a polar aprotic solvent, or a non-polar solvent.

The invention described herein also provides methods for generating a stapled peptide using a mono-metallated catalyst bearing a haloaryl group. Such methods provide an alternative non-symmetric synthesis of a stapled peptide. For example, such synthesis can occur in a stepwise manner, in which a first bond forming step occurs between a first cysteine residue in a peptide and a mono-metallated haloarylation reagent. A second cross-coupling step may then occur between a second cysteine residue and the aryl halide, yielding the target stapled peptide product.

In certain embodiments, the invention relates to a method, wherein said method is represented by Scheme 5:

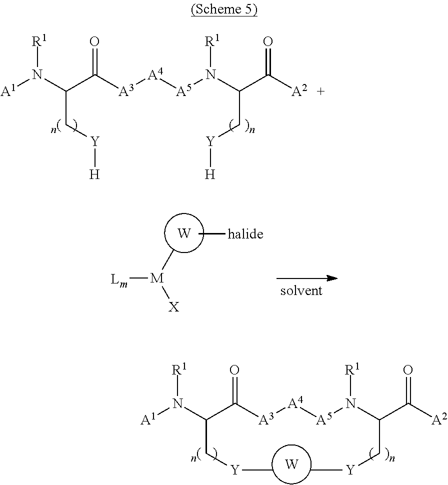

wherein, independently for each occurrence:

A¹ is H, an amine protecting group, alkyl, arylalkyl, acyl, aryl, alkoxycarbonyl, aryloxycarbonyl, a natural or unnatural amino acid, a plurality of natural amino acids or unnatural amino acids, a peptide, an oligopeptide, a polypeptide, a protein, an antibody, or an antibody fragment;

A² is NH₂, NH(amide protecting group), N(amide protecting group), OH, O(carboxylate protecting group), a natural or unnatural amino acid, a plurality of natural amino acids or unnatural amino acids, a peptide, an oligopeptide, a polypeptide, a protein, an antibody, or an antibody fragment;

A³, A⁴, and A⁵ are selected from the group consisting of a natural amino acid, an unnatural amino acid, and a plurality of natural amino acids or unnatural amino acids;

Y is S or Se;

R¹ is H, alkyl, arylalkyl, acyl, aryl, alkoxycarbonyl, aryloxycarbonyl, a natural or unnatural amino acid, a plurality of natural amino acids or unnatural amino acids, a peptide, an oligopeptide, a polypeptide, a protein, an antibody, or an antibody fragment;

M is Ni, Pd, Pt, Cu, or Au;

X is a halide, triflate, tetrafluoroborate, tetraarylborate, hexafluoroantimonate, bis(alkylsulfonyl)amide, tetrafluorophosphate, hexafluorophosphate, alkylsulfonate, haloalkylsulfonate, arylsulfonate, perchlorate, bis(fluoroalkylsulfonyl)amide, bis(arylsulfonyl)amide, (fluoroalkylsulfonyl)(fluoroalkyl-carbonyl)amide, nitrate, nitrite, sulfate, hydrogensulfate, alkyl sulfate, aryl sulfate, carbonate, bicarbonate, carboxylate, phosphate, hydrogen phosphate, dihydrogen phosphate, phosphinate, or hypochlorite;

L is independently for each occurrence a trialkylphosphine, a triarylphosphine, a dialkylarylphosphine, an alkyldiarylphosphine, an (alkenyl)(alkyl)(aryl)phosphine, an alkenyldiarylphosphine, an alkenyldialkylphosphine, a phosphine oxide, a bis(phosphine), a phosphoramide, a triarylphosphonate, an N-heterocyclic carbene, an optionally substituted phenanthroline, an optionally substituted iminopyridine, an optionally substituted 2,2′-bipyridine, an optionally substituted diimine, an optionally substituted triazolylpyridine, or an optionally substituted pyrazolyl pyridine;

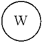

is aryl, heteroaryl, alkenyl, or cycloalkenyl, wherein

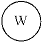

is optionally further substituted by one or more substituents selected from halide, acyl, azide, isothiocyanate, alkyl, aralkyl, alkenyl, alkynyl or protected alkynyl, alkoxyl, arylcarbonyl, cycloalkyl, formyl, haloalkyl, hydroxyl, amino, nitro, sulfhydryl, amido, phosphonate, phosphinate, alkylthio, sulfonyl, sulfonamido, heterocyclyl, aryl, heteroaryl, —CF₃, —CF₂R⁷, —CFR⁷₂, —CN, polyethylene glycol, polyethylene imine, —(CH₂)ₚ-FG-R⁷, and Z;

Z is

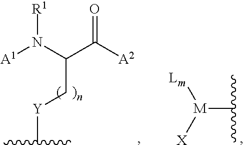

—S-alkyl, —SH, —S—(CH₂)ₙ—CO₂H, —SCH(CH₃)—CO₂H, or —SCH(CO₂H)—CH₂CO₂H;

p is independently for each occurrence an integer from 0-10;

FG is independently for each occurrence selected from the group consisting of C(O), CO₂, O(CO), C(O)NR⁷, NR⁷C(O), O, Si(R⁷)₂, C(NR⁷), (R⁷)₂N(CO)N(R⁷)₂, OC(O)NR⁷, NR⁷C(O)O, and C(N═N);

R⁷ is independently for each occurrence selected from the group consisting of H, alkyl, cycloalkyl, aryl, aralkyl, alkenyl, and alkynyl;

n is an integer from 1-5;

m is 1 or 2; and

solvent is a polar protic solvent, a polar aprotic solvent, or a non-polar solvent.

In certain embodiments, the invention relates to any one of the aforementioned methods, wherein the solvent is an inert solvent, preferably one in which the reaction ingredients, including the catalyst, are substantially soluble. Suitable solvents include ethers such as diethyl ether, 1,2-dimethoxyethane, diglyme, t-butyl methyl ether, tetrahydrofuran, water and the like; halogenated solvents such as chloroform, dichloromethane, dichloroethane, chlorobenzene, and the like; aliphatic or aromatic hydrocarbon solvents such as benzene, xylene, toluene, hexane, pentane and the like; esters and ketones, such as ethyl acetate, acetone, and 2-butanone; polar aprotic solvents, such as acetonitrile, dimethylsulfoxide, dimethylformamide and the like; or combinations of two or more solvents.

In certain embodiments, the invention relates to any one of the aforementioned methods, wherein the solvent is a solvent mixture. In certain embodiments, the solvent mixture is an aqueous solvent mixture including a polar aprotic solvent. In certain embodiments, the invention relates to any one of the aforementioned methods, wherein the solvent comprises water and a polar protic solvent such as acetonitrile, dimethylsulfoxide, or dimethylformamide. In certain embodiments, the solvent is a solvent mixture comprising water and acetonitrile. In certain embodiments, the invention relates to any one of the aforementioned methods, wherein the solvent is a solvent mixture comprising water and dimethylformamide. In certain embodiments, the solvent mixture comprises from about 20:1 water to polar aprotic solvent to about 1:20 water to polar aprotic solvent, about 19:1 water to polar aprotic solvent to about 1:19 water to polar aprotic solvent, or about 18:1 water to polar aprotic solvent to about 1:18 water to polar aprotic solvent. In certain embodiments, the solvent mixture comprises from about 5:1 water to polar aprotic solvent to about 1:5 water to polar aprotic solvent. In certain embodiments, the solvent mixture further comprises a buffer. For example, the buffer may be Tris, HEPES, MOPS, MES, or Na₂HPO₄:NaH₂PO₄. In certain embodiments, the concentration of the buffer is from about 0.01 M to about 1 M, for example, about 25 mM or about 0.1 M.

In certain embodiments, the invention relates to any one of the aforementioned methods, wherein the reaction takes place at from about 4° C. to about 40° C. In certain embodiments, the invention relates to any one of the aforementioned methods, wherein the reaction takes place at about 10° C., about 15° C., about 20° C., about 25° C., about 30° C., or about 35° C.

In certain embodiments, the invention relates to any one of the aforementioned methods, wherein the reaction is substantially complete after about 10 s, about 20 s, about 30 s, about 40 s, about 50 s, about 1 min, about 2 min, about 3 min, about 4 min, about 5 min, about 10 min, about 15 min, about 20 min, about 25 min, about 30 min, about 35 min, about 40 min, about 45 min, about 50 min, about 55 min, about 60 min, about 65 min, about 70 min, about 75 min, about 80 min, about 85 min, or about 90 min. In certain embodiments, the invention relates to any one of the aforementioned methods, wherein the reaction is substantially complete after about 2 h, about 3 h, about 4 h, about 5 h, about 6 h, about 7 h, about 8 h, about 9 h, about 10 h, about 11 h, or about 12 h.

The reactions of the present invention may be performed under a wide range of conditions, though it will be understood that the solvents and temperature ranges recited herein are not limitative and only correspond to exemplary modes of the processes of the invention.

In general, it will be desirable that reactions are run using mild conditions which will not adversely affect the reactants, the precatalyst, or the product. For example, the reaction temperature influences the speed of the reaction, as well as the stability of the reactants and catalyst. The reactions will usually be run at temperatures in the range of 20° C. to 300° C., more preferably in the range 20° C. to 150° C. In certain embodiments, the reactions will be run at room temperature (i.e., about 20° C. to about 25° C.). In certain embodiments, the pH of the reaction mixture may be about 8.5. In certain embodiments, the pH of the reaction mixture may be about 8.0, about 7.5, about 7.0, about 6.5, about 6.0, about 5.5, about 5.0, about 4.5, about 4.0, about 3.5, about 3.0, about 2.5, about 2.0, or about 1.5.

Another aspect of the invention relates to a method of functionalizing a thiol or selenol in a biopolymer, comprising contacting a biopolymer comprising a thiol or selenol moiety with a reagent of structural formula II, as defined above. The conditions under which the biopolymer and II come into contact with one another are sufficient to generate the functionalized biopolymer, in which Ar¹ is installed at the thiol or selenol moiety of the biopolymer. In certain embodiments, the biopolymer is an oligonucleotide, a polynucleotide, an oligosaccharide, or a polysaccharide.

In certain embodiments, the invention relates to a method of functionalizing a thiol or selenol in a biopolymer, wherein the functionalization reagent is a compound of formula (II) as described herein.

Another aspect of the invention relates to a method, comprising contacting a biopolymer comprising a first thiol moiety or a first selenol moiety and a second thiol or a second selenol moiety with a reagent of formula IV as defined herein, thereby generating a functionalized biopolymer, wherein the first thiol moiety or the first selenol moiety has been covalently bound to the second thiol moiety or the second selenol moiety by R^{y}. The conditions under which the biopolymer and IV come into contact with one another are sufficient to generate the functionalized biopolymer. In certain embodiments, the biopolymer is an oligonucleotide, a polynucleotide, an oligosaccharide, or a polysaccharide.

In certain embodiments, the invention relates to a method of functionalizing a thiol or selenol in a biopolymer, wherein the functionalization reagent is a compound of formula (IV) as described herein.

In certain embodiments of the method represented by Scheme 1, Ar¹ is (C₆-C₁₀)carbocyclic aryl, (C₃-C₁₂)heteroaryl, (C₃-C₁₄)polycyclic aryl, or alkenyl, substituted by one or more substituents independently selected from the group consisting of halide, acyl, azide, isothiocyanate, alkyl, aralkyl, alkenyl, alkynyl or protected alkynyl, alkoxyl, arylcarbonyl, cycloalkyl, formyl, haloalkyl, hydroxyl, amino, nitro, sulfhydryl, amido, phosphonate, phosphinate, alkylthio, sulfonyl, sulfonamido, heterocyclyl, aryl, heteroaryl, —CF₃, —CF₂R⁷, —CFR⁷₂, —CN, polyethylene glycol, polyethylene imine, and —(CH₂)ₚ-FG-R⁷;

p is independently for each occurrence an integer from 0-10;

FG is independently for each occurrence selected from the group consisting of C(O), CO₂, O(CO), C(O)NR⁷, NR⁷C(O), O, Si(R⁷)₂, C(NR⁷), (R⁷)₂N(CO)N(R⁷)₂, OC(O)NR⁷, NR⁷C(O)O, and C(N═N);

R⁷ is independently for each occurrence selected from the group consisting of H, alkyl, cycloalkyl, aryl, aralkyl, alkenyl, and alkynyl;

wherein at least one of the one or more substituents is halide.

Certain arylated products contain functional groups that allow for further functionalization of the product. In certain embodiments, an aryl-halide bond provides a useful handle for such further functionalization. For example, the aryl-halide bond can undergo a metal-catalyzed or metal-mediated cross-coupling reaction with an additional thiol-containing reagent.

Accordingly, in certain embodiments wherein Ar¹ is (C₆-C₁₀)carbocyclic aryl, (C₃-C₁₂)heteroaryl, (C₃-C₁₄)polycyclic aryl, or alkenyl substituted by at least one halide, the method represented by Scheme 1 further comprises contacting compound III,

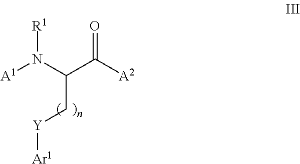

with a compound containing a thiol moiety or a selenol moiety; thereby yielding a coupling product.

In certain embodiments, the compound containing a thiol moiety or a selenol moiety is a small molecule having a molecular weight below about 500 g/mol.

In certain embodiments, the compound containing a thiol moiety or a selenol moiety is a biomolecule such as a natural or unnatural amino acid, a plurality of natural or unnatural amino acids, peptide, oligopeptide, polypeptide, or protein.

In certain embodiments, the step of contacting compound III with a compound containing a thiol moiety or a selenol moiety occurs in the presence of a Pd byproduct from the reaction depicted in Scheme 1.

### Exemplary Compounds

In certain embodiments, the invention relates to a compound comprising substructure III:

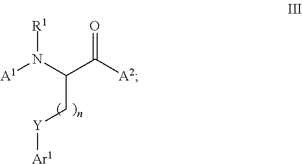

wherein,

A¹ is H, an amine protecting group, alkyl, arylalkyl, acyl, aryl, alkoxycarbonyl, aryloxycarbonyl, a natural or unnatural amino acid, a plurality of natural amino acids or unnatural amino acids, a peptide, an oligopeptide, a polypeptide, a protein, an antibody, or an antibody fragment;

A² is NH₂, NH(amide protecting group), N(amide protecting group), OH, O(carboxylate protecting group), a natural or unnatural amino acid, a plurality of natural amino acids or unnatural amino acids, a peptide, an oligopeptide, a polypeptide, a protein, an antibody, or an antibody fragment;

Y is S or Se;

R¹ is H, alkyl, arylalkyl, acyl, aryl, alkoxycarbonyl, or aryloxycarbonyl, a natural or unnatural amino acid, a plurality of natural amino acids or unnatural amino acids, a peptide, an oligopeptide, a polypeptide, a protein, an antibody, or an antibody fragment;

n is an integer from 1-5; and

Ar¹ is optionally substituted aryl, heteroaryl, alkenyl, or cycloalkenyl.

In certain embodiments, the invention relates to a compound comprising substructure III, wherein Ar¹ is covalently linked to a fluorophore, an imaging agent, a detection agent, a biomolecule, a therapeutic agent, a lipophilic moiety, a member of a high-affinity binding pair, or a cell-receptor targeting agent. In certain embodiments, the invention relates to any one of the aforementioned compounds, wherein Ar¹ is covalently linked to biotin. In certain embodiments, the invention relates to any one of the aforementioned compounds, wherein Ar¹ is covalently linked to fluorescein. In certain embodiments, the invention relates to any of the aforementioned compounds, wherein Ar¹ is covalently linked to a therapeutic agent; and the therapeutic agent is trametinib, topotecan, abiraterone, dabrafenib, or vandetanib.

In certain other embodiments, the invention relates to a compound comprising substructure III, wherein Ar¹ is comprised by a fluorophore. In certain embodiments, the invention relates to any of the aforementioned compounds, wherein Ar¹ is comprised by a therapeutic agent. In certain embodiments, the therapeutic agent is the trametinib, topotecan, abiraterone, dabrafenib, or vandetanib.

In certain embodiments, the fluorophore is a derivative of xanthene, fluorescein, rhodamine, coumarin, naphthalene, anathracene, oxadiazole, pyrene, acridine, tetrapyrrole, arylmethine, boron-dipyrromethene (BODIPY), or a cyanine dye. In certain other embodiments, the fluorophore is a fluorescent protein. In certain embodiments, the detection agent is for example, a nanoparticle, an MRI contrast agent, a dye moiety, or a radionuclide. In certain other embodiments, a biomolecule is a protein, a peptide, a monosaccharide, a disaccharide, a polysaccharide, a lipid, a glycolipid, a glycerolipid, a phospholipid, a hormone, a neurotransmitter, a nucleic acid, a nucleotide, a nucleoside, a sterol, a metabolite, a vitamin, or a natural product.

In certain embodiments, a therapeutic agent is a compound or substructure of a compound that brings about a therapeutic effect in a subject to which the agent is administered. In certain embodiments, the therapeutic agent is toxic to certain cells. Exemplary therapeutic agents that are covalently linked to Ar¹ in substructure III include trametinib, topotecan, abiraterone, dabrafenib, or vandetanib.

In certain embodiments, the lipophilic moiety enables the compound of substructure III to which the lipophilic moiety is conjugated to have an affinity for, or be soluble in, lipids, fats, oils, ad non-polar solvents, as described herein. Exemplary lipophilic moieties include amphiphilic surfactants, such as cinnamic acid.

In certain embodiments, the cell-receptor targeting agent is a ligand such as an epitope, a peptide, an antibody, a small organic compound, a neurotransmitter. High-affinity binding pairs include biotin-avidin, biotin-streptavidin, ligand-cell receptor, S-Peptide and Ribonuclease A, digoxigenin and its receptor, and complementary oligonucleotide pairs.

In certain embodiments, the invention relates to a compound comprising substructure III, wherein A¹ and A² are independently a natural or unnatural amino acid, a plurality of natural or unnatural amino acids, a peptide, an oligopeptide, a polypeptide, or a protein.

In certain embodiments, A¹ and A² of substructure III each independently comprise arginine, histidine, lysine, aspartic acid, glutamic acid, serine, threonine, asparagine, glutamine, proline, tyrosine, or tryptophan. In certain embodiments, A¹ and A² do not comprise cysteine or selenocysteine. In certain embodiments, A¹ and A² do not comprise any amino acids that contain —SH or —SeH moieties.

In certain embodiments, the invention relates to a compound comprising substructure III, wherein R¹ is H. In certain embodiments, the invention relates to a compound comprising substructure III, wherein X is halide, such as chloride. In certain embodiments, X is triflate.

In certain embodiments, the invention relates to a compound comprising substructure III, wherein A¹ and A² are covalently linked. In certain embodiments, substructure III comprises a cyclic peptide having an functionalized S moiety or a functionalized Se moiety. In certain embodiments, the functionalized S moiety or functionalized Se moiety is an arylated S moiety or an arylated Se moiety, respectively.

In certain embodiments, A¹ or A² comprises an antibody or an antibody fragment. In certain embodiments, the antibody is intact and comprises a single-point mutation with functionalized (e.g., arylated) Cys, Sec, or an artificial amino acid comprising —S(functional group) or —Se(functional group) on its main chain terminus. In alternative embodiments, A¹ or A² comprises an antibody fragment after partial antibody reduction.

In certain embodiments, the invention relates to any one of the compounds described herein.

### Exemplary Stapled Compounds

In certain embodiments, the invention relates to a compound comprising substructure V:

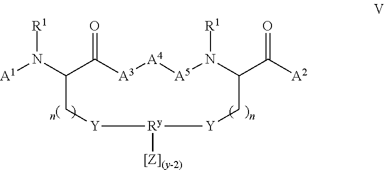

wherein, independently for each occurrence,

A¹ is H, an amine protecting group, alkyl, arylalkyl, acyl, aryl, alkoxycarbonyl, aryloxycarbonyl, a natural or unnatural amino acid, a plurality of natural amino acids or unnatural amino acids, a peptide, an oligopeptide, a polypeptide, a protein, an antibody, or an antibody fragment;

A² is NH₂, NH(amide protecting group), N(amide protecting group), OH, O(carboxylate protecting group), a natural or unnatural amino acid, a plurality of natural amino acids or unnatural amino acids, a peptide, an oligopeptide, a polypeptide, a protein, an antibody, or an antibody fragment;

A³, A⁴, and A⁵ are selected from the group consisting of a natural amino acid, an unnatural amino acid, and a plurality of natural amino acids or unnatural amino acids;

Y is S or Se;

n is 1-5;

R^{y} is an optionally substituted bridging moiety, comprising an aromatic group, a heteroaromatic group, an alkene group, or a cycloalkene group;

y is 2, 3, 4, 5, or 6;

each Z is independently

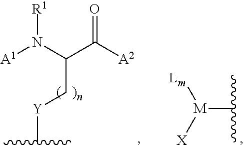

—S-alkyl, —SH, —S—(CH₂)ₙ—CO₂H, —SCH(CH₃)—CO₂H, or —SCH(CO₂H)—CH₂CO₂H; and

R¹ is H, alkyl, arylalkyl, acyl, aryl, alkoxycarbonyl, or aryloxycarbonyl, a natural or unnatural amino acid, a plurality of natural amino acids or unnatural amino acids, a peptide, an oligopeptide, a polypeptide, a protein, an antibody, or an antibody fragment.

In certain embodiments, the invention relates to any of the compounds described herein, wherein none of A¹, A², A³, A⁴, and A⁵ comprises cysteine.

In certain embodiments, the invention relates to any of the compounds described herein, wherein one or more of A¹, A², A³, A⁴, and A⁵ comprises arginine, histidine, lysine, aspartic acid, glutamic acid, serine, threonine, asparagine, glutamine, glycine, proline, alanine, valine, isoleucine, leucine, methionine, phenylalanine, tyrosine, or tryptophan.

In certain embodiments, the invention relates to any of the compounds described herein, wherein R^{y} is an optionally substituted bifunctional bridging moiety or an optionally substituted trifunctional bridging moiety.

In certain embodiments, the invention relates to any of the compounds described herein, wherein R^{y} comprises an aromatic group.

In certain embodiments, the invention relates to any of the compounds described herein, wherein R^{y} is optionally substituted

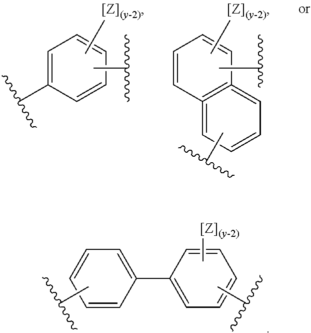

In certain embodiments, the invention relates to any of the compounds described herein, wherein R^{y} is not a perfluorinated aryl para-substituted diradical.

In certain embodiments, the invention relates to any one of the compounds described herein, wherein y is 2; and R^{y} is selected from the group consisting of

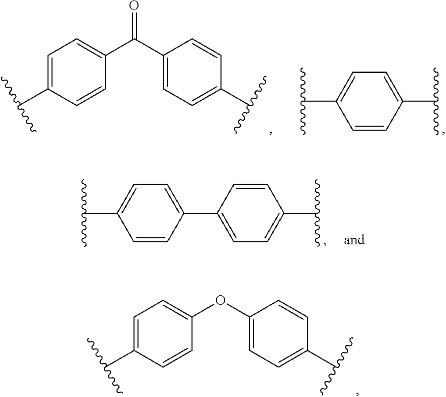

wherein any of the bifunctional bridging moieties may be optionally substituted.

In certain embodiments, the invention relates to a compound comprising substructure VI:

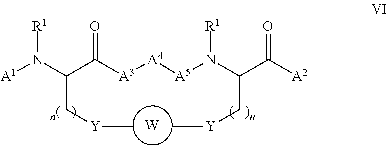

wherein, independently for each occurrence:

A¹ is H, an amine protecting group, alkyl, arylalkyl, acyl, aryl, alkoxycarbonyl, aryloxycarbonyl, a natural or unnatural amino acid, a plurality of natural amino acids or unnatural amino acids, a peptide, an oligopeptide, a polypeptide, a protein, an antibody, or an antibody fragment;

A² is NH₂, NH(amide protecting group), N(amide protecting group), OH, O(carboxylate protecting group), a natural or unnatural amino acid, a plurality of natural amino acids or unnatural amino acids, a peptide, an oligopeptide, a polypeptide, a protein, an antibody, or an antibody fragment;

A³, A⁴, and A⁵ are selected from the group consisting of a natural amino acid, an unnatural amino acid, and a plurality of natural amino acids or unnatural amino acids;

Y is S or Se;

R¹ is H, alkyl, arylalkyl, acyl, aryl, alkoxycarbonyl, aryloxycarbonyl, a natural or unnatural amino acid, a plurality of natural amino acids or unnatural amino acids, a peptide, an oligopeptide, a polypeptide, a protein, an antibody, or an antibody fragment;

M is Ni, Pd, Pt, Cu, or Au;

X is a halide, triflate, tetrafluoroborate, tetraarylborate, hexafluoroantimonate, bis(alkylsulfonyl)amide, tetrafluorophosphate, hexafluorophosphate, alkylsulfonate, haloalkylsulfonate, arylsulfonate, perchlorate, bis(fluoroalkylsulfonyl)amide, bis(arylsulfonyl)amide, (fluoroalkylsulfonyl)(fluoroalkyl-carbonyl)amide, nitrate, nitrite, sulfate, hydrogensulfate, alkyl sulfate, aryl sulfate, carbonate, bicarbonate, carboxylate, phosphate, hydrogen phosphate, dihydrogen phosphate, phosphinate, or hypochlorite;

L is independently for each occurrence a trialkylphosphine, a triarylphosphine, a dialkylarylphosphine, an alkyldiarylphosphine, an (alkenyl)(alkyl)(aryl)phosphine, an alkenyldiarylphosphine, an alkenyldialkylphosphine, a phosphine oxide, a bis(phosphine), a phosphoramide, a triarylphosphonate, an N-heterocyclic carbene, an optionally substituted phenanthroline, an optionally substituted iminopyridine, an optionally substituted 2,2′-bipyridine, an optionally substituted diimine, an optionally substituted triazolylpyridine, or an optionally substituted pyrazolyl pyridine;

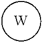

is aryl, heteroaryl, alkenyl, or cycloalkenyl, wherein

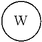

is optionally further substituted by one or more substituents selected from halide, acyl, azide, isothiocyanate, alkyl, aralkyl, alkenyl, alkynyl or protected alkynyl, alkoxyl, arylcarbonyl, cycloalkyl, formyl, haloalkyl, hydroxyl, amino, nitro, sulfhydryl, amido, phosphonate, phosphinate, alkylthio, sulfonyl, sulfonamido, heterocyclyl, aryl, heteroaryl, —CF₃, —CF₂R⁷, —CFR⁷₂, —CN, polyethylene glycol, polyethylene imine, —(CH₂)ₚ-FG-R⁷, and Z;

Z is

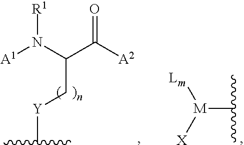

—S-alkyl, —SH, —S—(CH₂)ₙ—CO₂H, —SCH(CH₃)—CO₂H, or —SCH(CO₂H)—CH₂CO₂H;

p is independently for each occurrence an integer from 0-10;

FG is independently for each occurrence selected from the group consisting of C(O), CO₂, O(CO), C(O)NR⁷, NR⁷C(O), O, Si(R⁷)₂, C(NR⁷), (R⁷)₂N(CO)N(R⁷)₂, OC(O)NR⁷, NR⁷C(O)O, and C(N═N);

R⁷ is independently for each occurrence selected from the group consisting of H, alkyl, cycloalkyl, aryl, aralkyl, alkenyl, and alkynyl;

n is an integer from 1-5; and

m is 1 or 2;

In certain embodiments of the compound comprising substructure VI,

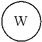

is selected from the group consisting of

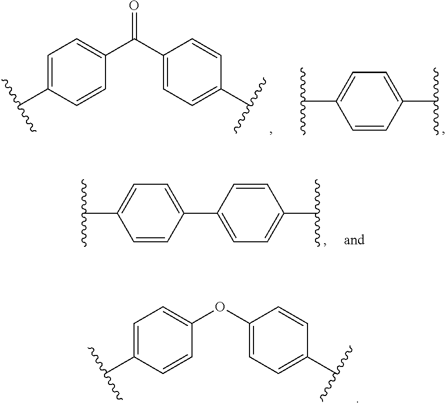

In certain embodiments, wherein A¹ and A² are independently a natural or unnatural amino acid, a plurality of natural or unnatural amino acids, a peptide, an oligopeptide, a polypeptide, or a protein.

In certain embodiments, A¹ comprises arginine, histidine, lysine, aspartic acid, glutamic acid, serine, threonine, asparagine, glutamine, proline, tyrosine, or tryptophan.

In certain embodiments, A² comprises arginine, histidine, lysine, aspartic acid, glutamic acid, serine, threonine, asparagine, glutamine, proline, tyrosine, or tryptophan.

In certain embodiments, A¹ and A² do not comprise cysteine or selenocysteine.

In certain embodiments, R¹ is H.

### Exemplary Polymetalated Reagents

In certain embodiments, the invention relates to a compound of formula IV:

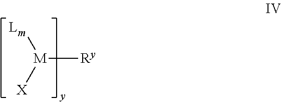

wherein, independently for each occurrence,

M is Ni, Pd, Pt, Cu, or Au;

R^{y} is an optionally substituted bridging moiety, comprising an aromatic group, a heteroaromatic group, an alkene group, or a cycloalkene group;

y is 2, 3, 4, 5, or 6;

X is a halide, triflate, tetrafluoroborate, tetraarylborate, hexafluoroantimonate, bis(alkylsulfonyl)amide, tetrafluorophosphate, hexafluorophosphate, alkylsulfonate, haloalkylsulfonate, arylsulfonate, perchlorate, bis(fluoroalkylsulfonyl)amide, bis(arylsulfonyl)amide, (fluoroalkylsulfonyl)(fluoroalkyl-carbonyl)amide, nitrate, nitrite, sulfate, hydrogensulfate, alkyl sulfate, aryl sulfate, carbonate, bicarbonate, carboxylate, phosphate, hydrogen phosphate, dihydrogen phosphate, phosphinate, or hypochlorite;

L is independently for each occurrence a trialkylphosphine, a triarylphosphine, a dialkylarylphosphine, an alkyldiarylphosphine, an (alkenyl)(alkyl)(aryl)phosphine, an alkenyldiarylphosphine, an alkenyldialkylphosphine, a phosphine oxide, a bis(phosphine), a phosphoramide, a triarylphosphonate, an N-heterocyclic carbene, an optionally substituted phenanthroline, an optionally substituted iminopyridine, an optionally substituted 2,2′-bipyridine, an optionally substituted diimine, an optionally substituted triazolylpyridine, or an optionally substituted pyrazolyl pyridine; and

m is 1 or 2.

In certain embodiments, the invention relates to any one of the compounds described herein, wherein R^{y} is an optionally substituted bifunctional bridging moiety or an optionally substituted trifunctional bridging moiety.

In certain embodiments, the invention relates to any one of the compounds described herein, wherein R^{y} comprises an aromatic group.

In certain embodiments, the invention relates to any one of the compounds described herein, wherein R^{y} is optionally substituted

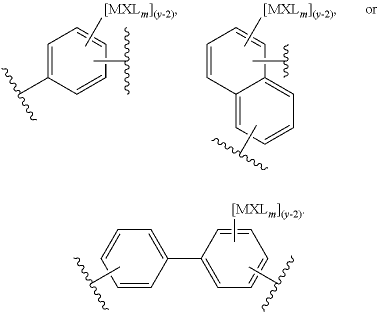

In certain embodiments, the invention relates to any one of the compounds described herein, wherein y is 2; and R^{y} is selected from the group consisting of

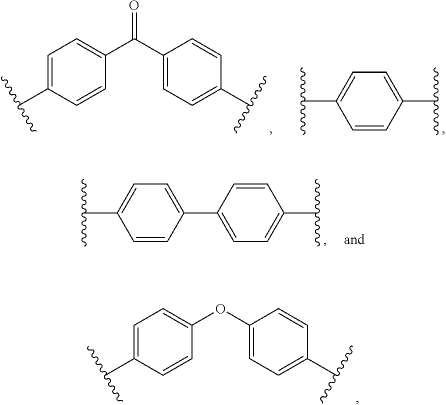

wherein any of the bifunctional bridging moieties may be optionally substituted.

In certain embodiments, the invention relates to any one of the compounds described herein, wherein y is 3; and R^{y} is selected from the group consisting of

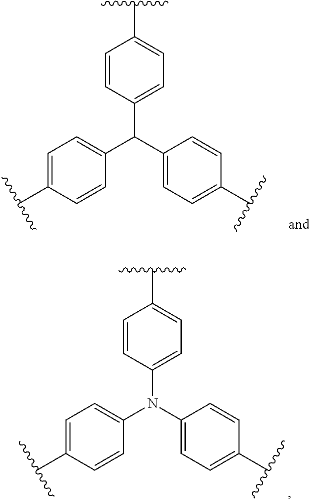 [SMILES: *c1ccc(C(c2ccc(*)cc2)c2ccc(*)cc2)cc1 ; *c1ccc(N(c2ccc(*)cc2)c2ccc(*)cc2)cc1 ; ","]

wherein any of the trifunctional bridging moieties may be optionally substituted.

### Exemplary Precatalysts and Methods

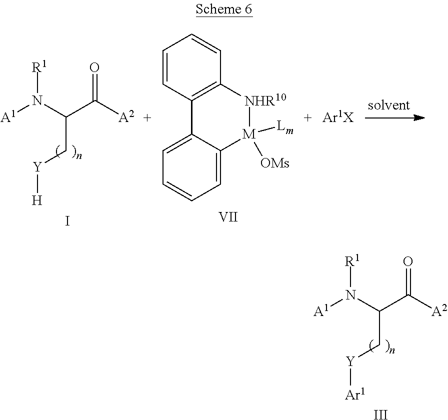

The invention also provides methods of functionalizing (e.g., arylating) a thiol or selenol (e.g., as in the representative reaction represented by Scheme 6) using a metal precatalyst in conjunction with Ar¹X (e.g., an aryl halide) reagent.

In certain embodiments, precatalysts exhibit the advantageous property of air stability. Exemplary precatalysts include Ph-mesylate palladium precatalysts (e.g., 2-amino biphenyl Pd species, such as the second generation Buchwald catalyst).

In embodiments of the reaction represented by Scheme 6,

Ar¹ is optionally substituted aryl, heteroaryl, alkenyl, or cycloalkenyl;

X is a halide, triflate, tetrafluoroborate, tetraarylborate, hexafluoroantimonate, bis(alkylsulfonyl)amide, tetrafluorophosphate, hexafluorophosphate, alkylsulfonate, haloalkylsulfonate, arylsulfonate, perchlorate, bis(fluoroalkylsulfonyl)amide, bis(arylsulfonyl)amide, (fluoroalkylsulfonyl)(fluoroalkyl-carbonyl)amide, nitrate, nitrite, sulfate, hydrogensulfate, alkyl sulfate, aryl sulfate, carbonate, bicarbonate, carboxylate, phosphate, hydrogen phosphate, dihydrogen phosphate, phosphinate, or hypochlorite;

R¹⁰ represents H, alkyl, alkenyl, alkynyl, cycloalkyl, cycloalkylalkyl, aralkyl, aryl, heteroaralkyl, or heteroaryl; and

A¹, A², R¹, Y, L, M, m, and n are as defined for Scheme 1.

### Exemplary Conjugated Compounds

In certain embodiments, the invention relates to a hybrid composition, wherein the hybrid composition comprises a linker, a compound of substructure III, and a detectable moiety; and the linker links the compound to the detectable moiety.

In certain embodiments, the invention relates to any one of the aforementioned hybrid compositions, wherein the detectable moiety is a fluorescent moiety, a dye moiety, a radionuclide, a drug molecule, an epitope, or an MRI contrast agent.

In certain embodiments, the invention relates to a hybrid composition, wherein the hybrid composition comprises a linker, a compound of substructure III, and a biomolecule; and the linker links the compound to the biomolecule.

In certain embodiments, the invention relates to any one of the aforementioned hybrid compositions, wherein the biomolecule is a protein.

In certain embodiments, the invention relates to any one of the aforementioned hybrid compositions, wherein the protein is an antibody.

In certain embodiments, the invention relates to any one of the aforementioned hybrid compositions, wherein the biomolecule is DNA, RNA, or peptide nucleic acid (PNA).

In certain embodiments, the invention relates to any one of the aforementioned hybrid compositions, wherein the biomolecule is siRNA.

In certain embodiments, the invention relates to a hybrid composition, wherein the hybrid composition comprises a linker, a compound of substructure III, and a polymer; and the linker links the compound to the polymer.

In certain embodiments, the invention relates to any one of the aforementioned hybrid compositions, wherein the polymer is polyethylene glycol.

In certain embodiments, the invention relates to any one of the hybrid compositions described herein.

### Exemplary Peptides, Oligopeptides, Polypeptides, and Proteins

In certain embodiments, the invention relates to a method to generate a peptide, an oligopeptide, a polypeptide, or a protein, wherein the peptide, oligopeptide, polypeptide, or protein comprises substructure III.

In certain embodiments, the invention relates to a peptide, an oligopeptide, a polypeptide, or a protein, wherein the peptide, oligopeptide, polypeptide, or protein comprises a plurality of substructures comprising substructure III.

In certain embodiments, the invention relates to any one of the peptides, oligopeptides, polypeptides, or proteins described herein.

In certain embodiments, the invention relates to a method to generate a peptide, an oligopeptide, a polypeptide, or a protein, wherein the peptide, oligopeptide, polypeptide, or protein comprises substructure V.

In certain embodiments, the invention relates to a peptide, an oligopeptide, a polypeptide, or a protein, wherein the peptide, oligopeptide, polypeptide, or protein comprises a plurality of substructures comprising substructure V.

In certain embodiments, the invention relates to a method to generate a peptide, an oligopeptide, a polypeptide, or a protein, wherein the peptide, oligopeptide, polypeptide, or protein comprises substructure VI.

In certain embodiments, the invention relates to a peptide, an oligopeptide, a polypeptide, or a protein, wherein the peptide, oligopeptide, polypeptide, or protein comprises a plurality of substructures comprising substructure VI.

In certain embodiments, the invention relates to a peptide, an oligopeptide, a polypeptide, or a protein, or a method involving the peptide, oligopeptides, polypeptide, or protein, described in US published patent application publication number US 2014/0113871, which is hereby incorporated by reference in its entirety.

### Exemplary Therapeutic Methods

Antibody-drug conjugates (ADCs) are an emerging class of anti-cancer therapeutics. Highly cytotoxic small molecule drugs are conjugated to antibodies to create a single molecular entity. ADCs combine the high efficacy of small molecules with the target specificity of antibodies to enable the selective delivery of drug payloads to cancerous tissues, which reduces the systematic toxicity of conventional small molecule drugs.

Traditionally, ADCs are prepared by conjugating small molecule drugs to either cysteines generated from reducing an internal disulfide bond or surface-exposed lysines. Because multiple lysines and cysteines are present in antibodies, these conventional approaches usually lead to heterogeneous products with undefined drug-antibody ratio, which might cause difficulty for manufacturing and characterization. Furthermore, each individual antibody-drug conjugate may exhibit different pharmacokinetics, efficacy, and safety profiles, hindering a rational approach to optimizing ADC-based cancer treatment.

Recent studies showed that ADCs prepared using site-specific conjugation techniques exhibited improved pharmacological profiles.

So, in certain embodiments, the invention relates to an ADC with defined position of drug-attachment and defined drug to antibody ratio. In certain embodiments, the ADCs of the invention permit rational optimization of ADC-based therapies. In certain embodiments, the ADC comprises a structure of any one of the compounds generated by the methods described herein. In certain embodiments, the drug-to-antibody ratio is about 2:1, about 3:1, about 4:1, about 5:1, about 6:1, about 7:1, about 8:1, about 9:1, about 10:1, about 11:1, or about 12:1.

In certain embodiments, the invention relates to any one of the ADCs mentioned herein, comprising monomethyl auristatin E (MMAE) covalently conjugated to an antibody, wherein the antibody targets a cell surface receptor that is over-expressed in a cancer cell. MMAE is a highly toxic antimitotic agent that inhibits cell division by blocking tubulin polymerization. MMAE has been successfully conjugated to antibodies targeting human CD30 to create ADCs that have been approved by FDA to treat Hodgkin lymphoma as well as anaplastic large-cell lymphoma. In certain embodiments, the invention relates to a method for the selective synthesis of an ADC comprising MMAE covalently conjugated to an antibody.

In certain embodiments, the invention relates to any one of the ADCs mentioned herein, wherein the antibody targets cell receptors CD30, CD22, CD33, human epidermal growth factor receptor 2 (HER2), or epidermal growth factor receptor (EGFR). It should be noted that by conjugating drugs to antibodies targeting different receptors, the ADCs prepared should be useful for treating different cancers.

### Definitions

For convenience, before further description of the present invention, certain terms employed in the specification, examples, and appended claims are collected here.

The articles “a” and “an” are used herein to refer to one or to more than one (i.e., to at least one) of the grammatical object of the article. By way of example, “an element” means one element or more than one element.

The term “heteroatom” is art-recognized and refers to an atom of any element other than carbon or hydrogen. Illustrative heteroatoms include boron, nitrogen, oxygen, phosphorus, sulfur and selenium.

The term “alkyl” refers to the radical of saturated aliphatic groups, including straight-chain alkyl groups, branched-chain alkyl groups, cycloalkyl (alicyclic) groups, alkyl substituted cycloalkyl groups, and cycloalkyl substituted alkyl groups. In preferred embodiments, a straight chain or branched chain alkyl has 30 or fewer carbon atoms in its backbone (e.g., C₁-C₃₀ for straight chain, C₃-C₃₀ for branched chain), and more preferably 20 or fewer. Likewise, preferred cycloalkyls have from 3-10 carbon atoms in their ring structure, and more preferably have 5, 6 or 7 carbons in the ring structure.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths but with at least two carbon atoms. Preferred alkyl groups are lower alkyls. In preferred embodiments, a substituent designated herein as alkyl is a lower alkyl.

The term “aralkyl”, as used herein, means an aryl group, as defined herein, appended to the parent molecular moiety through an alkyl group, as defined herein. Representative examples of arylalkyl include, but are not limited to, benzyl, 2-phenylethyl, 3-phenylpropyl, and 2-naphth-2-ylethyl.

The term “alkoxy” means an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

The term “alkoxycarbonyl” means an alkoxy group, as defined herein, appended to the parent molecular moiety through a carbonyl group, represented by —C(═O)—, as defined herein. Representative examples of alkoxycarbonyl include, but are not limited to, methoxycarbonyl, ethoxycarbonyl, and tert-butoxycarbonyl.

The term “carboxy” as used herein, means a —CO₂H group.

The term “alkylthio” as used herein, means an alkyl group, as defined herein, appended to the parent molecular moiety through a sulfur atom. Representative examples of alkylthio include, but are not limited, methylthio, ethylthio, tert-butylthio, and hexylthio. The terms “arylthio,” “alkenylthio” and “arylakylthio,” for example, are likewise defined.

The term “amido” as used herein, means —NHC(═O)—, wherein the amido group is bound to the parent molecular moiety through the nitrogen. Examples of amido include alkylamido such as CH₃C(═O)N(H)— and CH₃CH₂C(═O)N(H)—.

The term “aryl” as used herein includes 5-, 6- and 7-membered aromatic groups that may include from zero to four heteroatoms, for example, benzene, naphthalene, anthracene, pyrene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics”. The aromatic ring can be substituted at one or more ring positions with such substituents as described above, for example, halogen, azide, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings can be cycloalkyls, cycloalkenyls, cycloalkynyls, aryls and/or heterocyclyls.

The abbreviations Me, Et, Ph, Tf, Nf, Ts, Ms, and dba represent methyl, ethyl, phenyl, trifluoromethanesulfonyl, nonafluorobutanesulfonyl, p-toluenesulfonyl, methanesulfonyl, and dibenzylideneacetone, respectively. Also, “DCM” stands for dichloromethane; “rt” stands for room temperature, and may mean about 20° C., about 21° C., about 22° C., about 23° C., about 24° C., about 25° C., or about 26° C.; “THF” stands for tetrahydrofuran; “BINAP” stands for 2,2′-bis(diphenylphosphino)-1,1′-binaphthyl; “dppf” stands for 1,1′-bis(diphenylphosphino)ferrocene; “dppb” stands for 1,4-bis(diphenylphosphinobutane; “dppp” stands for 1,3-bis(diphenylphosphino)propane; “dppe” stands for 1,2-bis(diphenylphosphino)ethane. A more comprehensive list of the abbreviations utilized by organic chemists of ordinary skill in the art appears in the first issue of each volume of the Journal of Organic Chemistry; this list is typically presented in a table entitled Standard List of Abbreviations. The abbreviations contained in said list, and all abbreviations utilized by organic chemists of ordinary skill in the art are hereby incorporated by reference.

The terms ortho, meta and para apply to 1,2-, 1,3- and 1,4-disubstituted benzenes, respectively. For example, the names 1,2-dimethylbenzene and ortho-dimethylbenzene are synonymous.

The terms “heterocyclyl” or “heterocyclic group” refer to 3- to 10-membered ring structures, more preferably 3- to 7-membered rings, whose ring structures include one to four heteroatoms. Heterocycles can also be polycycles. Heterocyclyl groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring can be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The term “non-coordinating anion” relates to a negatively charged moiety that interacts weakly with cations. Non-coordinating anions are useful in studying the reactivity of electrophilic cations, and are commonly found as counterions for cationic metal complexes with an unsaturated coordination sphere. In many cases, non-coordinating anions have a negative charge that is distributed symmetrically over a number of electronegative atoms. Salts of these anions are often soluble non-polar organic solvents, such as dichloromethane, toluene, or alkanes.

The terms “polycyclyl” or “polycyclic group” refer to two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The term “heteroatom” as used herein means an atom of any element other than carbon or hydrogen. Preferred heteroatoms are nitrogen, oxygen, sulfur and phosphorous.

As used herein, the term “nitro” means —NO₂; the term “halogen” or “halo” designates —F, —Cl, —Br or —I; the term “sulfhydryl” means —SH; the term “hydroxyl” means —OH; the term “sulfonyl” means —SO₂—; and the term “cyano” as used herein, means a —CN group.

The term “haloalkyl” means at least one halogen, as defined herein, appended to the parent molecular moiety through an alkyl group, as defined herein. Representative examples of haloalkyl include, but are not limited to, chloromethyl, 2-fluoroethyl, trifluoromethyl, pentafluoroethyl, and 2-chloro-3-fluoropentyl.

The terms “amine” and “amino” are art recognized and refer to both unsubstituted and substituted amines, e.g., a moiety that can be represented by the general formula:

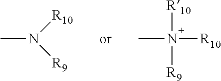

wherein R₉, R₁₀ and R′₁₀ each independently represent a hydrogen, an alkyl, an alkenyl, —(CH₂)ₘ—R₈, or R₉ and R₁₀ taken together with the N atom to which they are attached complete a heterocycle having from 4 to 8 atoms in the ring structure; R₈ represents an aryl, a cycloalkyl, a cycloalkenyl, a heterocycle or a polycycle; and m is zero or an integer in the range of 1 to 8. In preferred embodiments, only one of R₉ or R₁₀ can be a carbonyl, e.g., R₉, R₁₀ and the nitrogen together do not form an imide. In even more preferred embodiments, R₉ and R₁₀ (and optionally R′₁₀) each independently represent a hydrogen, an alkyl, an alkenyl, or —(CH₂)ₘ—R₈. Thus, the term “alkylamine” as used herein means an amine group, as defined above, having a substituted or unsubstituted alkyl attached thereto, i.e., at least one of R₉ and R₁₀ is an alkyl group.

The definition of each expression, e.g., alkyl, m, n, and the like, when it occurs more than once in any structure, is intended to be independent of its definition elsewhere in the same structure.

The terms triflyl (-Tf), tosyl (-Ts), mesyl (-Ms), and nonaflyl are art-recognized and refer to trifluoromethanesulfonyl, p-toluenesulfonyl, methanesulfonyl, and nonafluorobutanesulfonyl groups, respectively. The terms triflate (-OTf), tosylate (-OTs), mesylate (-OMs), and nonaflate are art-recognized and refer to trifluoromethanesulfonate ester, p-toluenesulfonate ester, methanesulfonate ester, and nonafluorobutanesulfonate ester functional groups and molecules that contain said groups, respectively.

The phrase “protecting group” as used herein means temporary modifications of a potentially reactive functional group which protect it from undesired chemical transformations. Examples of such protecting groups include silyl ethers of alcohols, and acetals and ketals of aldehydes and ketones, respectively. In embodiments of the invention, a carboxylate protecting group masks a carboxylic acid as an ester. In certain other embodiments, an amide is protected by an amide protecting group, masking the —NH₂ of the amide as, for example, —NH(alkyl), or —N(alkyl)₂. The field of protecting group chemistry has been reviewed (Greene, T. W.; Wuts, P. G. M. Protective Groups in Organic Synthesis, 2^{nd} ed.; Wiley: New York, 1991).

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc.

As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described hereinabove. The permissible substituents can be one or more and the same or different for appropriate organic compounds. For purposes of this invention, the heteroatoms, such as nitrogen, may have hydrogen substituents and/or any permissible substituents of organic compounds described herein which satisfy the valencies of the heteroatoms.

A “polar protic solvent” as used herein is a solvent having a dipole moment of about 1.4 to 4.0 D, and comprising a chemical moiety that participates in hydrogen bonding, such as an O—H bond or an N—H bond. Exemplary polar protic solvents include methanol, ethanol, n-propanol, isopropanol, n-butanol, isobutanol, ammonia, water, and acetic acid.

A “polar aprotic solvent” as used herein means a solvent having a dipole moment of about 1.4 to 4.0 D that lacks a hydrogen bonding group such as O—H or N—H. Exemplary polar aprotic solvents include acetone, N,N-dimethylformamide, acetonitrile, ethyl acetate, dichloromethane, tetrahydrofuran, and dimethylsulfoxide.

A “non-polar solvent” as used herein means a solvent having a low dielectric constant (<5) and low dipole moment of about 0.0 to about 1.2. Exemplary nonpolar solvents include pentane, hexane, cyclohexane, benzene, toluene, chloroform, and diethyl ether.

For purposes of this invention, the chemical elements are identified in accordance with the Periodic Table of the Elements, CAS version, Handbook of Chemistry and Physics, 67th Ed., 1986-87, inside cover.

### EXEMPLIFICATION

The invention may be understood with reference to the following examples, which are presented for illustrative purposes only and which are non-limiting. The substrates utilized in these examples were either commercially available, or were prepared from commercially available reagents.

### General Reagent Information

Tris(2-carboxyethyl)phosphine hydrochloride (TCEP.HCl) was purchased from Hampton Research (Aliso Viejo, Calif.). 1-[Bis(dimethylamino)methylene]-1H-1,2,3-triazolo[4,5-b]pyridinium 3-oxid hexafluorophosphate (HATU), D-Biotin, Fmoc-Rink amide linker, Fmoc-L-Gly-OH, Fmoc-L-Leu-OH, Fmoc-L-Lys(Boc)-OH, Fmoc-L-Ala-OH, Fmoc-L-Cys(Trt)-OH, Fmoc-L-Gln(Trt)-OH, Fmoc-L-Asn(Trt)-OH, Fmoc-L-Glu(OtBu)-OH, Fmoc-L-Arg(Pbf)-OH, Fmoc-L-Phe-OH, Fmoc-L-Ser(tBu)-OH, Fmoc-L-Thr(tBu)-OH, Fmoc-L-Tyr(tBu)-OH, and Fmoc-L-His(Trt)-OH were purchased from Chem-Impex International (Wood Dale, Ill.). Aminomethyl polystyrene resin was prepared according to an in-house protocol.¹ Peptide synthesis-grade N,N-dimethylformamide (DMF), dichloromethane (DCM), diethyl ether, HPLC-grade acetonitrile, and guanidine hydrochloride were obtained from VWR International (Philadelphia, Pa.). Aryl halides and aryl trifluoromethanesulfonates were purchased from Aldrich Chemical Co., Alfa Aesar, or Matrix Scientific and were used without additional purification. All deuterated solvents were purchased from Cambridge Isotopes and used without further purification. All other reagents were purchased from Sigma-Aldrich and used as received. Trastuzumab was a kind gift from Prof K. Dane Wittrup at MIT.

All reactions with peptides, proteins, and antibodies were set up on the bench top and carried out under ambient conditions. For procedures carried out in the nitrogen-filled glovebox, the dry degassed THF was obtained by passage through activated alumina columns followed by purging with argon. Anhydrous pentane, cyclohexane, and acetonitrile were purchased from Aldrich Chemical Company in Sureseal® bottles and were purged with argon before use.

### General Analytical Information

All small-molecule organic and organometallic compounds were characterized by ¹H, ¹³C NMR, and IR spectroscopy, as well as elemental analysis (unless otherwise noted). ¹⁹F NMR spectroscopy was used for organometallic complexes containing a trifluoromethanesulfonate counterion. ³¹P NMR spectroscopy was used for characterization of palladium complexes. Copies of the ¹H, ¹³C, ³¹P, and ¹⁹F NMR spectra can be found at the end of the Supporting Information. Nuclear Magnetic Resonance spectra were recorded on a Bruker 400 MHz instrument and a Varian 300 MHz instrument. Unless otherwise stated, all ¹H NMR experiments are reported in 6 units, parts per million (ppm), and were measured relative to the signals of the residual proton resonances CH₂Cl₂ (5.32 ppm) or CH₃CN (1.94 ppm) in the deuterated solvents. All ¹³C NMR spectra are measured decoupled from ¹H nuclei and are reported in δ units (ppm) relative to CD₂Cl₂ (54.00 ppm) or CD₃CN (118.69 ppm), unless otherwise stated. All ³¹P NMR spectra are measured decoupled from ¹H nuclei and are reported relative to H₃PO₄ (0.00 ppm). ¹⁹F NMR spectra are measured decoupled from ¹H nuclei and are reported in ppm relative to CFCl₃ (0.00 ppm) or α,α,α-trifluorotoluene (˜63.72 ppm). All FT-IR spectra were recorded on a Thermo Scientific—Nicolet iS5 spectrometer (iD5 ATR—diamond). Elemental analyses were performed by Atlantic Microlabs Inc., Norcross, Ga.

### LC-MS Analysis

LC-MS chromatograms and associated mass spectra were acquired using Agilent 6520 ESI-Q-TOF mass spectrometer. Solvent compositions used in the majority of experiments are 0.1% TFA in H₂O (solvent A) and 0.1% TFA in acetonitrile (solvent B). The following LC-MS methods were used:

Method A

LC conditions: Zorbax SB C₃ column: 2.1×150 mm, 5 m, column temperature: 40° C., gradient: 0-3 min 5% B, 3-22 min 5-95% B, 22-24 min 95% B, flow rate: 0.8 mL/min. MS conditions: positive electrospray ionization (ESI) extended dynamic mode in mass range 300-3000 m/z, temperature of drying gas=350° C., flow rate of drying gas=11 L/min, pressure of nebulizer gas=60 psi, the capillary, fragmentor, and octupole rf voltages were set at 4000, 175, and 750, respectively.

Method B

LC conditions: Zorbax SB C₃ column: 2.1×150 mm, 5 m, column temperature: 40° C., gradient: 0-2 min 5% B, 2-11 min 5-65% B, 11-12 min 65% B, flow rate: 0.8 mL/min. MS conditions are same as Method A.

Method C

LC conditions: Zorbax SB C₃ column: 2.1×150 mm, 5 m, column temperature: 40° C., gradient: gradient: 0-2 min 5% B, 2-10 min 5-95% B, 10-11 min 95% B, flow rate: 0.8 mL/min. MS conditions are same as Method A.

Data were processed using Agilent MassHunter software package. Deconvoluted masses of proteins were obtained using maximum entropy algorithm.

LC-MS data shown were acquired using Method A, unless otherwise noted; Y-axis in all chromatograms shown in supplementary figures represents total ion current (TIC); mass spectrum insets correspond to the integration of the TIC peak unless otherwise noted.

### Determination of Reaction Yields

All reported yields were determined by integrating TIC spectra. First, the peak areas for all relevant peptide-containing species on the chromatogram were integrated using Agilent MassHunter software package. Since no peptide-based side products were generated in the experiments, the yields shown in Table 2 were determined as follows: % yield=Sₚᵣ/Sₜₒₜₐₗ where Sₚᵣ is the peak area of the product and Sₜₒₜₐₗ is the peak area of combined peptide-containing species (product and starting material). The yield of the stapled peptide (Example 19) was calculated as follows: % yield=k·Sₚᵣ/Sₛₜ where Sₚᵣ is the peak area of the reaction product, Sₛₜ is the peak area of a known amount of purified product, and k equals to the ratio of the known amount of standard divided by the initial amount of starting material. For peptide stability experiments the conversion was calculated as following: % remaining peptide=Sₜ/S₀ where Sₜ is the peak area of the corresponding cysteine conjugate at time t, and S₀ is the peak area of the cysteine conjugate at time 0.

### Example 1—Preparation of Arylation Reagents

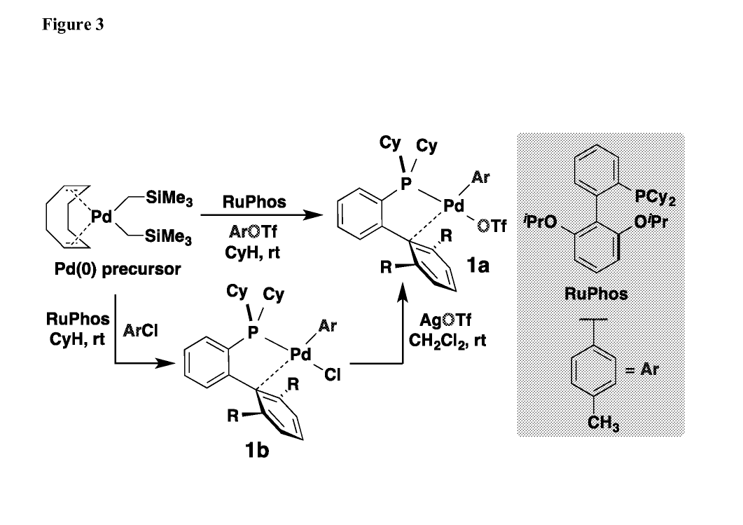

A series of Pd(II) reagents were designed that were capable of selectively recognizing a Cys moiety and transferring an aryl group. These reagents feature a biaryl phosphine ligand, which confers a bulky steric and electron-rich environment at the metal center favoring facile oxidative addition of electrophilic substrates. For example, complexes 1a-b were isolated as air-stable solids and were conveniently synthesized from the Pd(0) precursor and a phosphine ligand in the presence of aryl triflate or chloride electrophiles, respectively (FIG. 3). In an alternative synthesis, the triflate species was prepared from chloride complex 1b by salt metathesis with the Ag(I) salt. Overall, these synthetic transformations provide several complementary routes to a wide range of Pd(II) based reagents.

### Example 2—Model Polypeptide

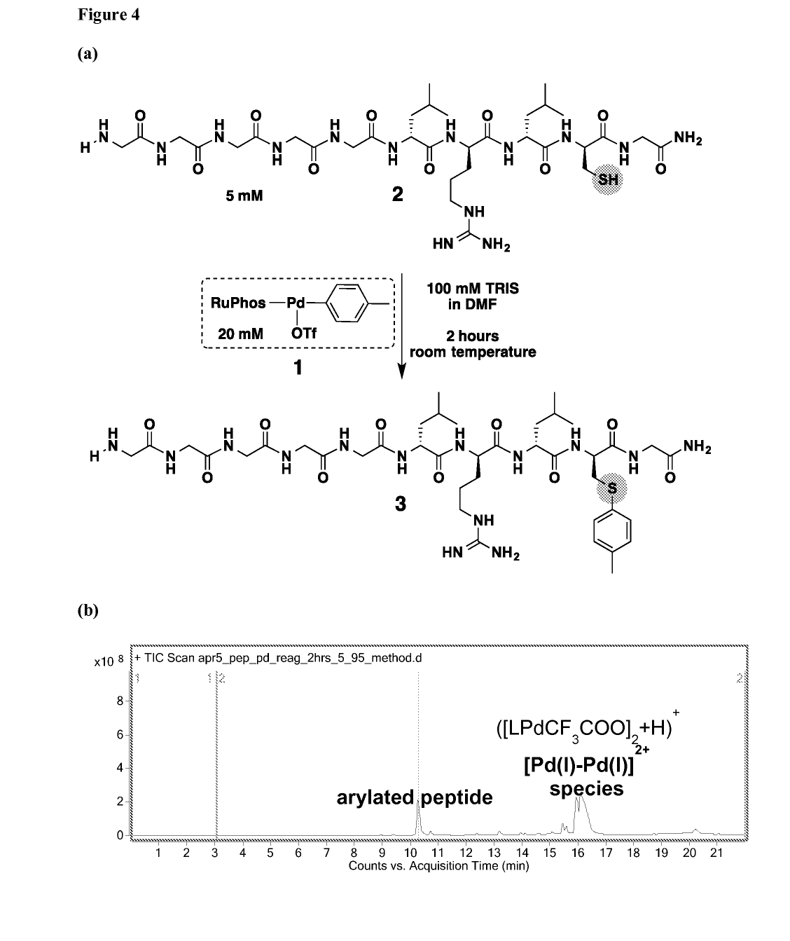

Reaction between 1a and a unprotected model polypeptide 2 (FIG. 4) resulted in a complete conversion of the starting peptide material as suggested by LC-MS analysis of the reaction mixture. Importantly, only Cys S-arylated product 3 was observed as a result of this transformation in combination with several decomposition products of 1 produced upon quenching with acid present in the LC-MS running solvent mixture. These decomposition products were identified as an arylated RuPhos phosphonium salt and a ligated Pd(I)-Pd(I) dimer species, both of which eluted significantly later relatively to the peptide product 3. The Pd(I)-Pd(I) dimer by-product was prepared independently and structurally characterized via NMR spectroscopy in solution and single-crystal X-ray diffraction in the solid-state to confirm its identity in the reaction mixture.

### Example 3—Control Peptides

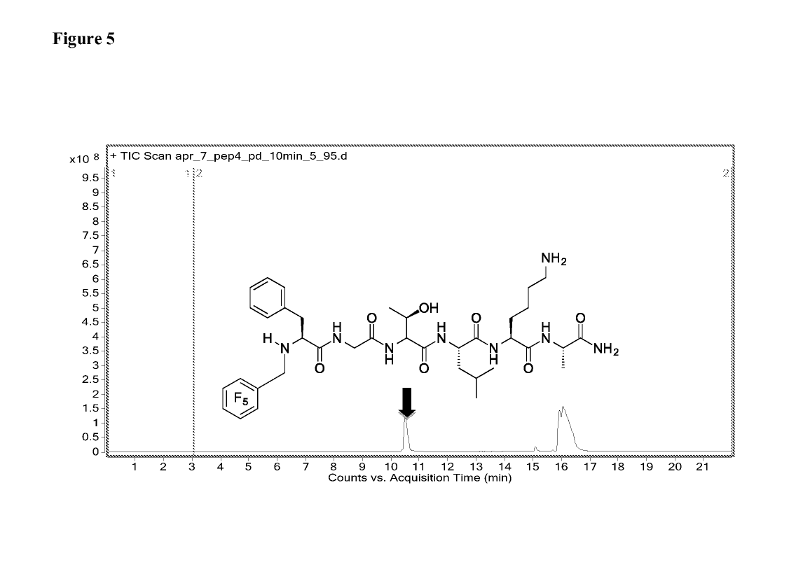

Control peptides lacking Cys residue or Sec residues were submitted to arylation conditions. For example, AKLTGF-NH(CH₂C₆F₅) and VTLPSTF*GAS showed no conversion and/or decomposition, indicating that arylation occurs exclusively on the Cys or Sec residue. The LCMS trace for AKLTGF-NH(CH₂C₆F₅) under arylation conditions is shown in FIG. 5.

### Example 4—Variation of Reaction Conditions

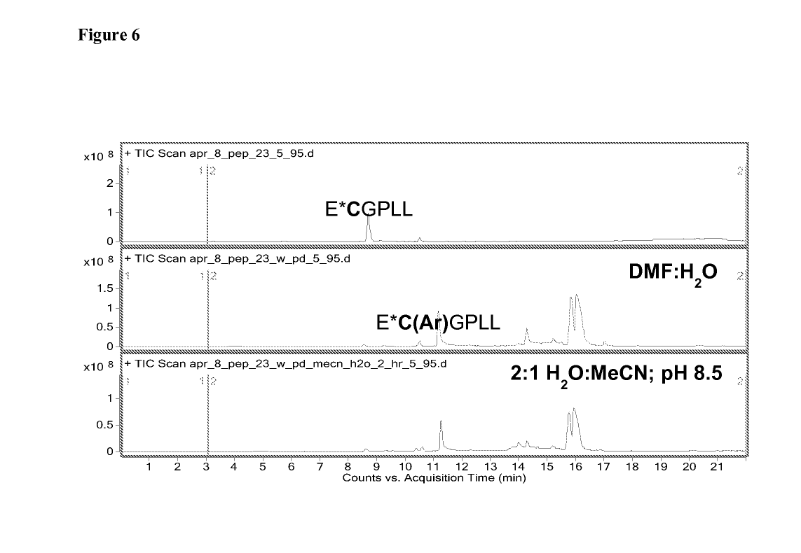

The Cys arylation described herein also operates in solvent mixtures containing water. Arylation experiments were conducted between a model peptide 4 (γ-Glu-Cys-Gly-Pro-Leu-Leu) and reagent 1a in 1:1 DMF:H₂O and 2:1 H₂O:MeCN mixtures, respectively. In both cases, selective transformation producing S-arylated peptide 5 (γ-Glu-CysTol-Gly-Pro-Leu-Leu) occurred within minutes suggesting very fast reaction kinetics (FIG. 6).

### Example 5—Functional Group Tolerance

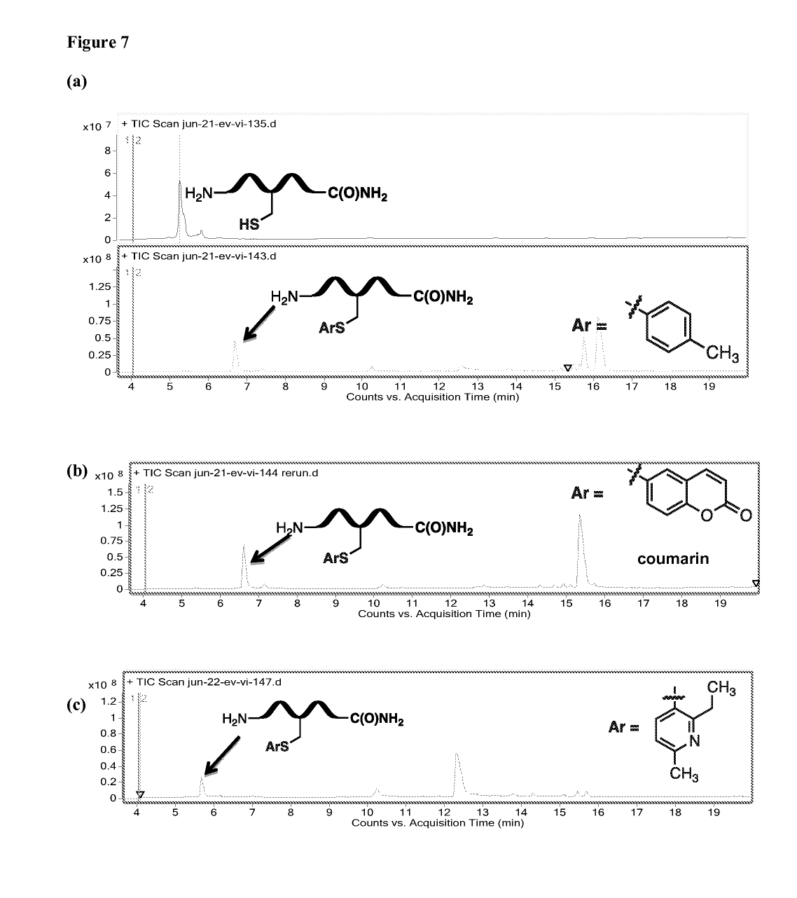

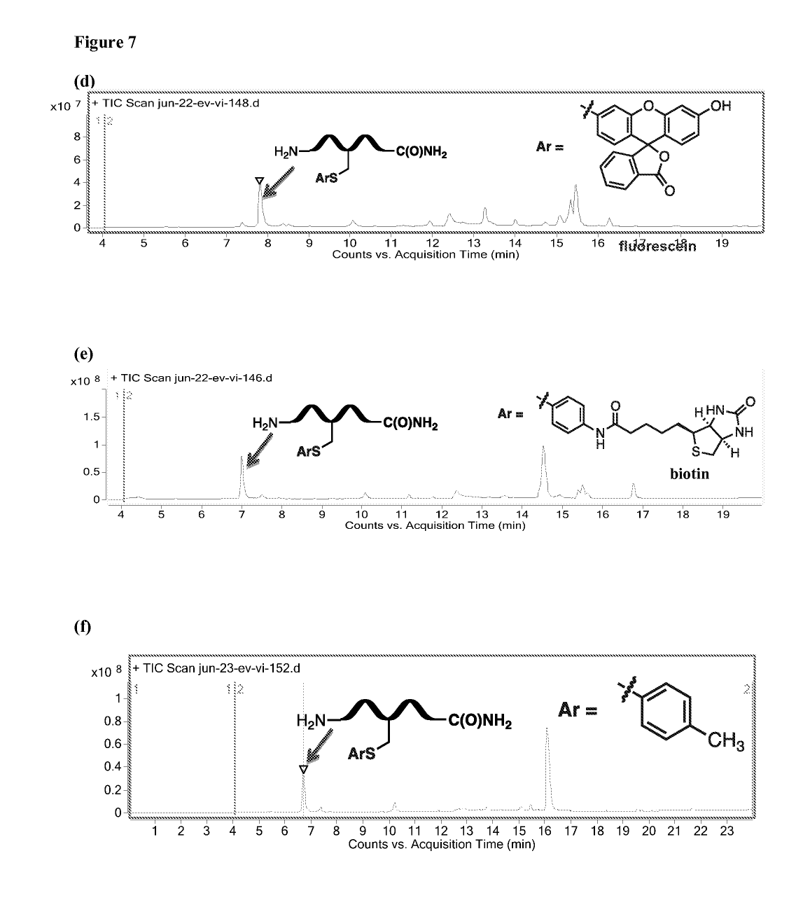

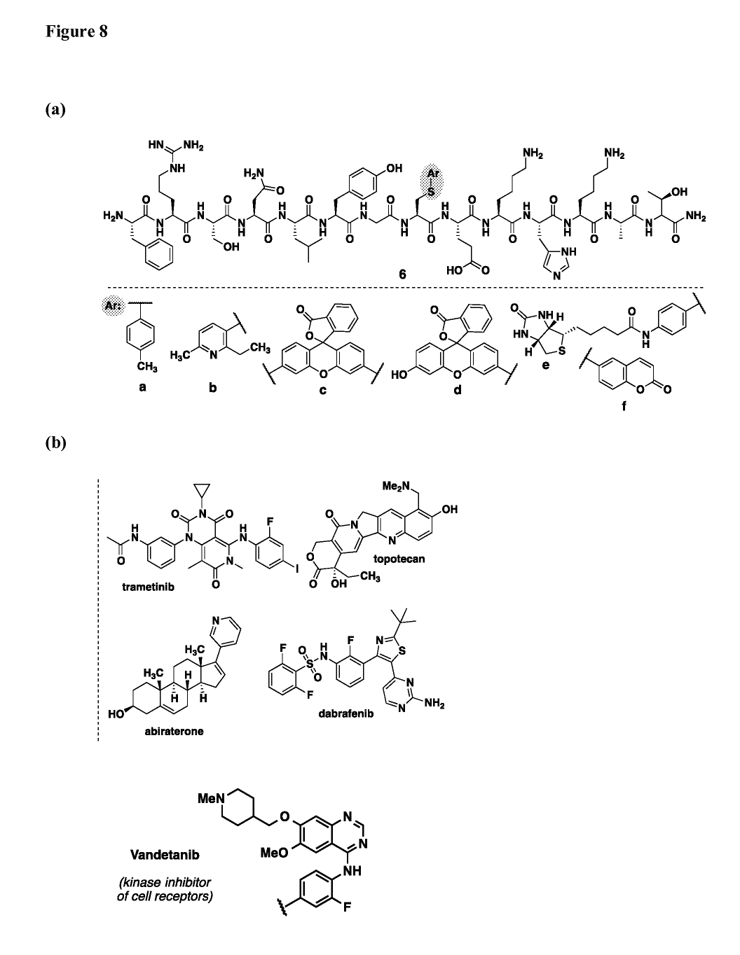

To address functional group tolerance of this transformation, studies were conducted between several Pd-based triflate reagents and the unprotected peptide 6 (FRSNLYGCEKHKAT-NH₂) featuring other common nucleophilic amino-acid residues such as OH (e.g., Tyr, Ser, Thr) and NH/NH₂ (e.g., His, Lys, Arg). Arylation reactions were conducted in the presence of 0.1 M Tris at a pH of 8.5, a solvent system of 1:2 CH₃CN:H₂O for 5 minutes, unless noted otherwise. For all arylation agents examined (6a-f), selective and nearly quantitative S-arylation was observed irrespective of the nature of the Pd(II) reagent used (FIG. 7). Furthermore, studies with Pd-based species 1b containing a chloride ligand instead of the triflate showed similar reactivity at 1 mM peptide concentration, producing S-arylated peptide 6a in 5 minutes. The arylation strategy is also amenable to bioconjugation with Pd(II) species containing complex drug molecules (FIG. 8).

### Example 6—Model Protein

The arylation chemistry was next evaluated using a model protein species containing a single Cys residue. DARPin protein with a single-point mutation incorporating a Cys residue on the N-terminus of the sequence chain was designed for these studies and expressed in E. coli (final amino-acid sequence: GGCGGSDLGKKLLEAARAGQDDEVRILMANGADVNAY DDNGVTPLHLAAFLGHLEIVEVLLKYGADVNAADSWGTTPLHLAATWGHLEIVEV LLKHGADVNAQDKFGKTAFDISIDNGNEDLAEILQKLN). A reaction between 50 uM protein with 5 equivalents of 1a resulted in a complete consumption of the starting material within 5 minutes. The resulting product mixture was analyzed by LC-MS confirming quantitative monoarylation of the protein.

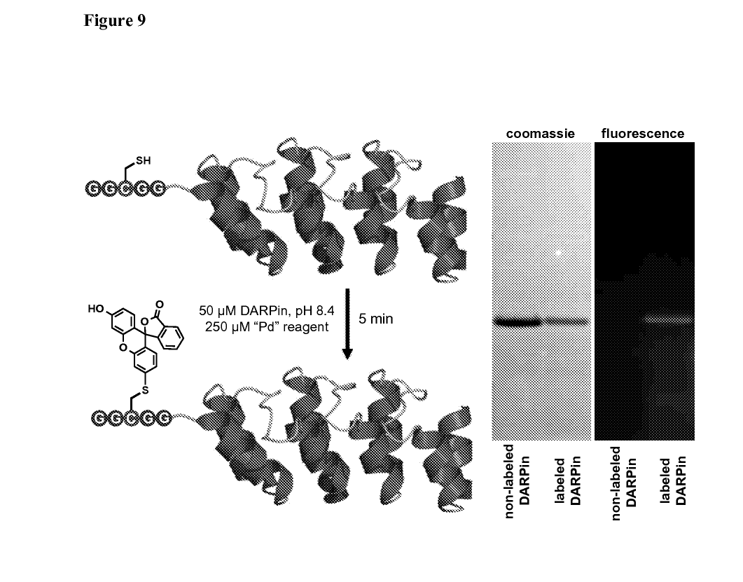

Trypsin digestion followed by MS/MS analysis of the product mixture indicated that the modification occurred exclusively on the Cys residue further corroborating results obtained with the peptide substrates (vide supra). In addition to reagent 1a, Cys arylation was successfully performed using other reagents, including biotinylated and fluorescein-based species. For example, reaction between DARPin and the Pd(II) reagent containing fluorescein resulted in a quantitative formation of an S-labeled protein species (FIG. 9). SDS-PAGE analysis of the reaction mixture confirmed fluorescent label incorporation (FIG. 9).

### Example 7—Cys-S-Arylation in Antibodies

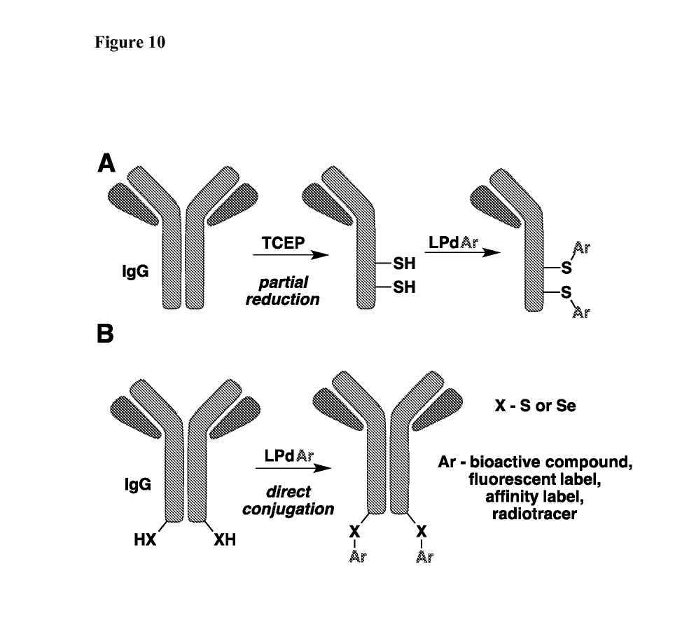

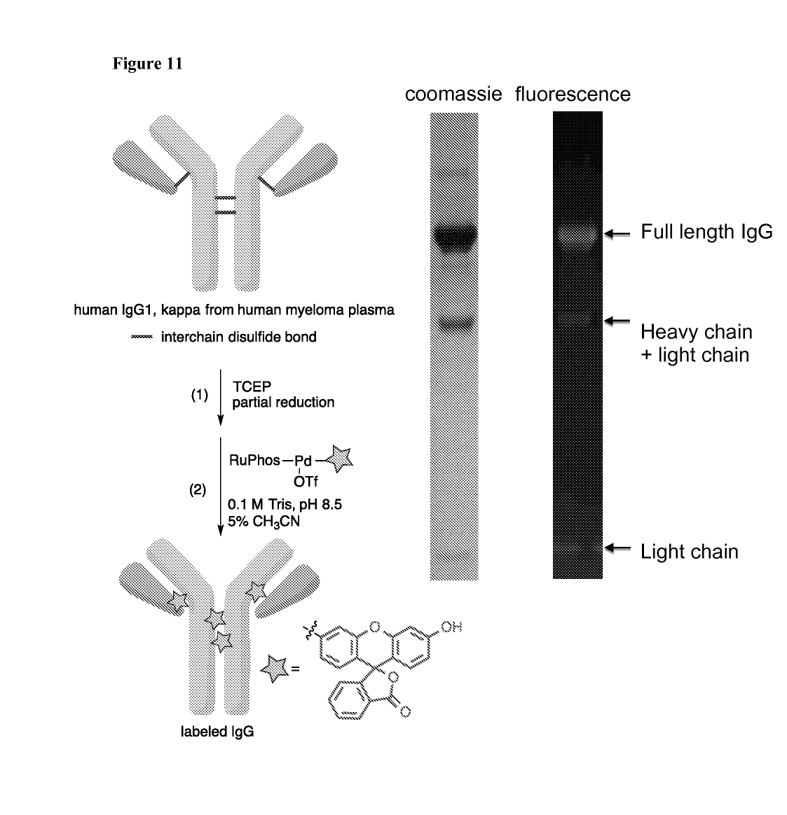

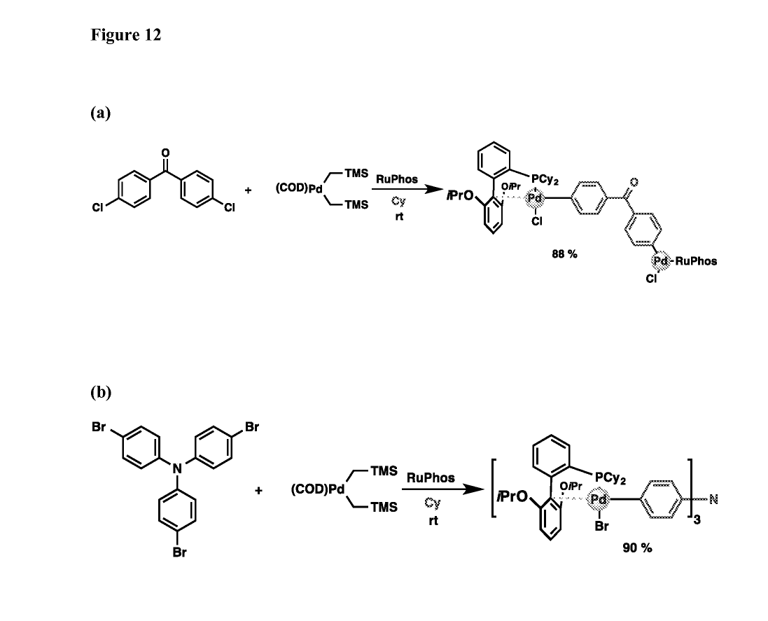

Further studies were aimed at functionalization of native and non-native Cys residues in IgG antibodies. Specifically, two independent approaches were examined, where one can either functionalize native Cys moieties after partial antibody reduction or perform functionalization on the intact antibody containing single-point mutation with Cys or selenocysteine moieties on the main-chain terminus (FIG. 10). In both cases, the resulting constructs are significantly more chemically stable towards degradation than their alkyl, disulfide and maleimide congeners. This stability enhancement along with the highly selective and rapid bioconjugation conferred by Pd(II) reagents should provide significantly improved handling capabilities and expanded therapeutic properties for the resulting antibody-drug conjugates. FIG. 11 shows a further S-arylation scheme in a human IgG1 antibody substrate, using fluorescein as arylation moiety. Reaction conditions (1) were conducted at 0.75 mg/mL IgG, 0.1 M Tris, 15 mM TCEP, pH 8.5, room temperature, 2 hours. Reaction conditions (2) were conducted at 0.5 mg/mL partially reduced IgG, 0.1 M Tris, 100 mM of Pd reagent, 5% acetonitrile, pH 8.5, 30 min at room temperature.

### Example 8—Synthesis of Palladium Reagents

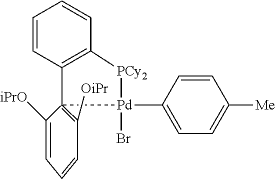

In a nitrogen-filled glovebox, an oven-dried scintillation vial (10 mL), which was equipped with a magnetic stir bar and fitted with a Teflon screwcap septum, was charged with RuPhos (66 mg, 0.14 mmol), 4-bromotoluene (24.2 mg, 0.14 mmol), and cyclohexane (1.0 mL). Solid (COD)Pd(CH₂SiMe₃)₂ (50.0 mg, 0.13 mmol) was added rapidly in one portion and the resulting solution was stirred for 16 h at rt. After this time, pentane (3 mL) was added and the resulting mixture was placed into a −20° C. freezer for 3 h. The vial was then taken outside of the glovebox, and the resulting precipitate was filtered, washed with pentane (3×3 mL), and dried under reduced pressure to afford the oxidative addition complex (78.4 mg, 82%).

¹H NMR (400 MHz, CD₂Cl₂) δ 7.61 (m, 2H), 7.43 (tt, J=7.5, 1.6 Hz, 1H), 7.37 (m, 1H), 6.91 (dd, J=8.2, 2.3 Hz, 2H), 6.86 (ddd, J=7.8, 3.1, 1.5 Hz, 1H), 6.76 (d, J=8.0 Hz, 2H), 6.64 (d, J=8.4 Hz, 2H), 4.60 (hept, J=6.1 Hz, 2H), 2.22 (s, 3H), 2.14 (m, 2H), 1.77 (m, 6H), 1.60 (m, 6H), 1.38 (d, J=6.0 Hz, 6H), 1.17 (m, 6H), 1.01 (d, J=6.0 Hz, 6H), 0.78 (m, 2H).

¹³C NMR (101 MHz, CD₂Cl₂) δ 159.42, 145.38, 145.20, 137.74, 137.70, 134.88, 134.18, 133.84, 133.11, 133.01, 132.94, 131.62, 131.56, 130.99, 130.97, 128.20, 126.81, 126.76, 112.44, 112.41, 107.88, 71.44, 34.40, 34.14, 28.73, 28.17, 28.15, 27.82, 27.69, 27.49, 27.46, 27.35, 26.60, 22.46, 21.93, 20.79 (observed complexity is due to C—P coupling).

³¹P NMR (121 MHz, CD₂Cl₂) δ 29.89.

### Example 9—Cysteine Arylation

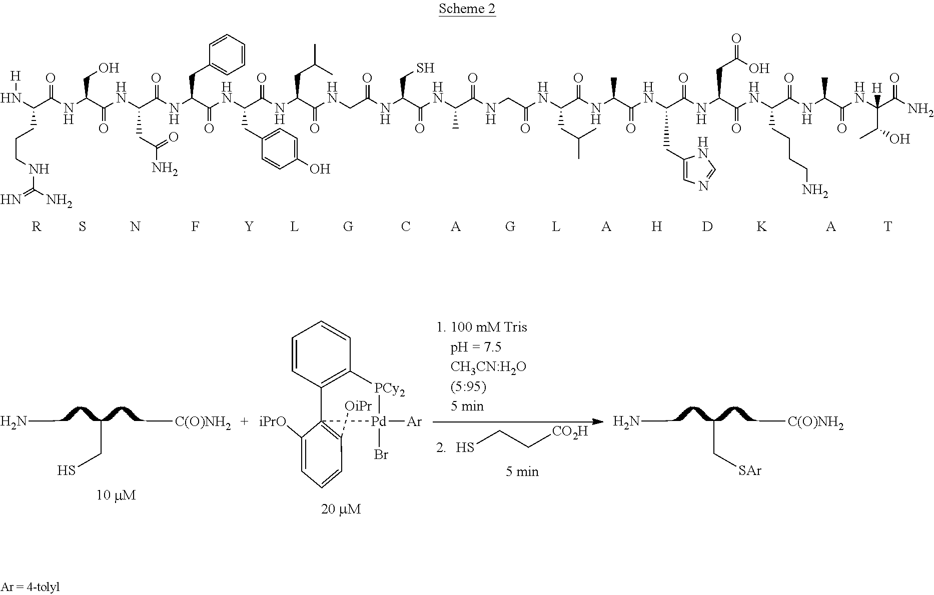

Peptide P1 (4 μL, 150 μM, above), H₂O (47 μL), organic solvent (1 μL) and the buffer (6 μL, 1 M) were combined in a 0.6 mL plastic Eppendorf tube and the resulting solution was mixed using a vortexer. A stock solution of the palladium complex (2 μL, 600 μM) in organic solvent was added in one portion, the reaction tube was vortexed to ensure proper reagent mixing and left at room temperature for 5 min. The reaction was quenched by the addition of 3-mercaptopropionic acid (6.3 μL, 0.05 μL/mL solution). After an additional 5 min the LCMS solution (60 μL) was added to the Eppendorf and the reaction mixture was analyzed by LCMS.

Final concentration of the reaction before quenching:

Peptide—10 μM,

Pd-complex—20 μM,

Tris buffer—100 mM;

CH₃CN:H₂O=5:95.

### Example 10—Synthesis of Polymetallic Species

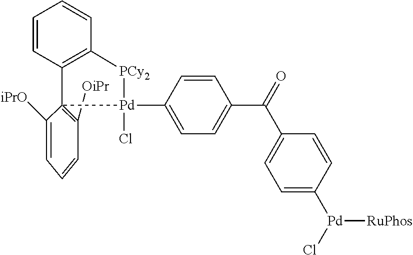

In a nitrogen-filled glovebox, an oven-dried scintillation vial (10 mL), which was equipped with a magnetic stir bar and fitted with a Teflon screwcap septum, was charged with RuPhos (139.4 mg, 0.30 mmol, 2.5 equiv), 4,4′-dichlorobenzophenone (30.0 mg, 0.12 mmol, 1 equiv) and cyclohexane (1.2 mL). Solid (COD)Pd(CH₂SiMe₃)₂ (116.2 mg, 0.30 mmol, 2.5 equiv) was added rapidly in one portion and the resulting solution was stirred for 16 h at rt. After this time, pentane (3 mL) was added and the resulting mixture was placed into a −20° C. freezer for 3 h. The vial was then taken outside of the glovebox, and the resulting precipitate was filtered, washed with pentane (3×3 mL), and dried under reduced pressure to afford the oxidative addition complex.

¹H NMR (400 MHz, CD₂Cl₂) δ 7.64 (m, 4H), 7.45 (m, 2H), 7.39 (m, 2H), 7.32 (d, J=8.0 Hz, 4H), 7.25 (dd, J=8.4, 2.1 Hz, 4H), 6.88 (ddd, J=7.7, 3.1, 1.3 Hz, 2H), 6.65 (d, J=8.5 Hz, 4H), 4.64 (hept, J=6.1 Hz, 4H), 2.14 (m, 4H), 1.70 (m, 24H), 1.39 (d, J=6.0 Hz, 12H), 1.20 (m, 12H), 1.02 (d, J=6.0 Hz, 12H), 0.75 (m, 4H).

¹³C NMR (101 MHz, CD₂Cl₂) δ 197.01, 159.78, 149.09, 145.47, 145.30, 137.25, 137.21, 135.49, 134.06, 133.94, 133.58, 133.06, 132.95, 131.55, 131.23, 131.21, 128.34, 126.98, 126.92, 111.50, 107.69, 71.53, 34.39, 34.12, 28.78, 28.32, 27.73, 27.59, 27.38, 27.27, 26.59, 22.44, 21.89 (observed complexity is due to C—P coupling).

³¹P NMR (121 MHz, CD₂Cl₂) δ 33.27.

### Example 11—Stapling

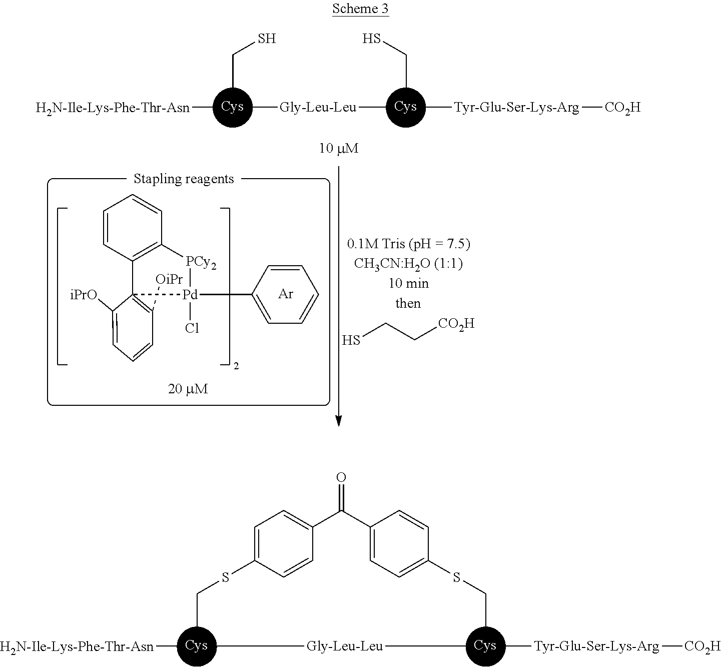

Peptide (4 μL, 150 μM), H₂O (23 μL), and Tris buffer (3 μL, 1 M, pH=7.5) were combined in a 0.6 mL plastic Eppendorf tube and the resulting solution was mixed using a vortexer. A stock solution of the palladium complex (30 μL, 40 μM) in CH₃CN was added in one portion, the reaction tube was vortexed to ensure proper reagent mixing and left at room temperature for 10 min. The reaction was quenched by the addition of 3-mercaptopropionic acid (6.3 μL, 0.1 μL/mL solution). After an additional 5 min the LCMS solution (60 μL) was added to the Eppendorf and the reaction mixture was analyzed by LCMS.

Final concentration of the reaction before quenching:

peptide—10 μM,

OA—20 μM,

Tris buffer—100 mM;

CH₃CN:H₂O=1:1.

### Example 12—Conjugating Drug Molecules to Antibody by Palladium Reagents

Conjugation protocol: Trastuzumab was partially reduced with TCEP on a 20-μL scale. Reaction conditions: 10 μM trastuzumab (˜1.5 mg/mL), 30 μM TCEP, 0.1 M Tris, pH 8.0, 37° C., 2 hours.

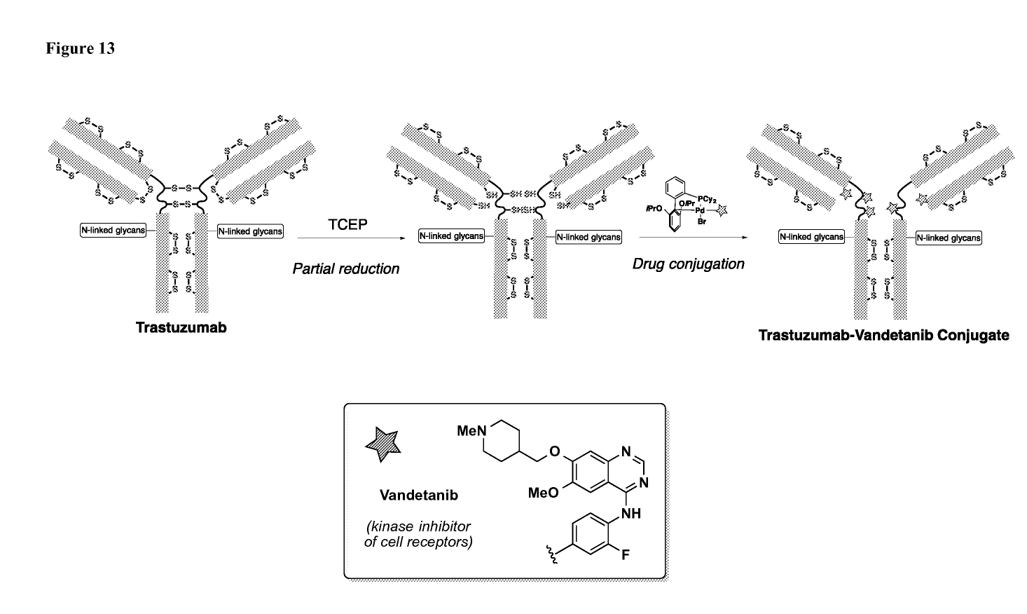

1 μL of 0.4 mM palladium-vandetanib complex dissolved in DMF was added to 20 μL of partially reduced antibody, the resulting mixture was left at room temperature for 30 minutes. See FIG. 13.

LC-MS analysis: 20 μL of crude reaction mixture was quenched by addition of 1 μL of 4 mM mercaptopropionic acid. The resulting solution was left at room temperature for 5 minutes, and was then buffer exchanged into buffer P (20 mM Tris, 150 mM NaCl, pH 7.5) using a 10K spin concentrator. N-linked glycans were removed by addition of 1 μL of PNGase F (New England Biolabs) was added to 100 μg of antibody and incubation at 45° C. for 1 hour. The resulting solution was completely reduced by addition of 1/10 volume of 200 mM TCEP solution (pH 7.5) and incubation at 37° C. for 30 minutes before subjecting to LC-MS analysis. Based on this analysis, the drug-to-antibody ratio (DAR) was calculated to be about 5.5 (data not shown).

### Example 13—Synthesis of Oxidative Additional Complexes

### General Procedure for the Synthesis of Oxidative Addition Complexes.

In a nitrogen-filled glovebox, an oven-dried scintillation vial (10 mL), which was equipped with a magnetic stir bar, was charged with RuPhos (1.1 equiv), Ar—X (1.1 equiv), and cyclohexane. Solid (COD)Pd(CH₂SiMe₃)₂ (McAtee, J. R. Angew. Chem., Int. Ed. 51, 3663-3667 (2012)) (1 equiv) was added rapidly in one portion and the resulting solution was stirred for 16 h at rt. After this time, pentane (3 mL) was added and the resulting mixture was placed into a −20° C. freezer for 3 h. The vial was then taken outside of the glovebox, and the resulting precipitate was filtered, washed with pentane (3×3 mL), and dried under reduced pressure to afford the oxidative addition complex.

### Exemplary Oxidative Addition Complexes

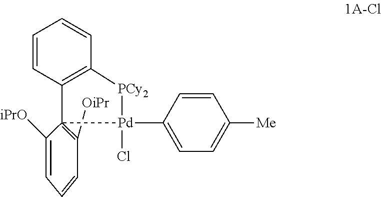

Following the general procedure, a mixture containing 4-chlorotoluene (17 μL, 0.14 mmol), RuPhos (66 mg, 0.14 mmol), and (COD)Pd(CH₂SiMe₃)₂ (50 mg, 0.13 mmol) was stirred at rt in cyclohexane (1.5 mL) for 16 h. General work up afforded 1A-CI as a white solid (68.7 mg, 77%).

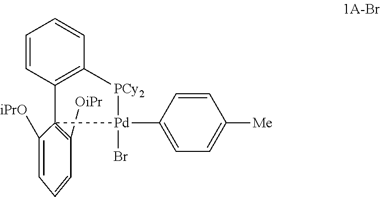

Following the general procedure, a mixture containing 4-bromotoluene (24.2 mg, 0.14 mmol), RuPhos (66.0 mg, 0.14 mmol), and (COD)Pd(CH₂SiMe₃)₂ (50.0 mg, 0.13 mmol) was stirred at rt in cyclohexane (1 mL) for 16 h. General work up afforded 1A-Br as an off-white solid (78.4 mg, 82%).

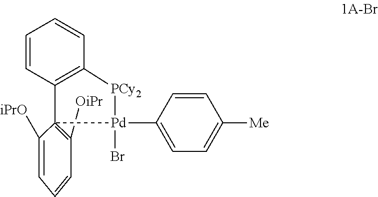

Following the general procedure, a mixture containing 4-iodotoluene (61.7 mg, 0.28 mmol), RuPhos (131.9 mg, 0.28 mmol), and (COD)Pd(CH₂SiMe₃)₂ (100.0 mg, 0.26 mmol) was stirred at rt in cyclohexane (1.5 mL) for 16 h. General work up afforded 1A-I as a bright yellow solid (180.0 mg, 89%).

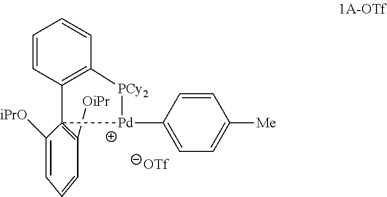

Following the general procedure, a mixture containing 4-tolyl trifluoromethanesulfonate (100.0 mg, 0.42 mmol), RuPhos (194.0 mg, 0.42 mmol), and (COD)Pd(CH₂SiMe₃)₂ (147.0 mg, 0.38 mmol) was stirred at rt in cyclohexane (1.5 mL) for 16 h. General work up afforded 1A-OTf as an off-white solid (270.0 mg, 88%).

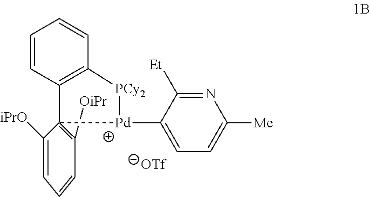

Following the general procedure, a mixture containing 2-ethyl-6-methylpyridin-3-yl trifluoromethanesulfonate (76.0 mg, 0.28 mmol, Note: 2.2 equiv was used), RuPhos (66.0 mg, 0.141 mmol), and (COD)Pd(CH₂SiMe₃)₂ (50.0 mg, 0.129 mmol) was stirred at rt in cyclohexane (0.75 mL) for 16 h. General work up afforded 1B as a light yellow solid (95.0 mg, 88%).

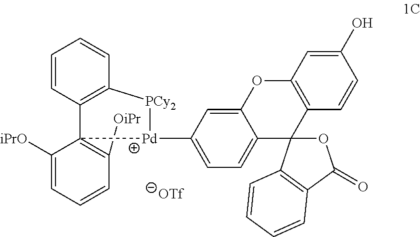

Following the general procedure, a mixture containing fluorescein monotrifluoromethanesulfonate (52.5 mg, 0.11 mmol, Note: used as the limiting reagent), RuPhos (66.0 mg, 0.14 mmol), and (COD)Pd(CH₂SiMe₃)₂ (50.0 mg, 0.13 mmol) was stirred in THF (0.75 mL) at rt for 16 h using aluminum foil for light exclusion. General work up afforded 1C as a bright orange precipitate (107.5 mg, 92%).

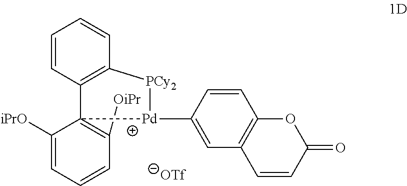

Following the general procedure, a mixture containing 2-oxo-2H-chromen-6-yl trifluoromethanesulfonate (38.2 mg, 0.13 mmol, Note: 1.01 equiv was used), RuPhos (66.0 mg, 0.14 mmol), and (COD)Pd(CH₂SiMe₃)₂ (50.0 mg, 0.13 mmol) was stirred at rt in THF (0.75 mL) for 16 h. General work up afforded 1D as a light yellow solid (103.3 mg, 93%).

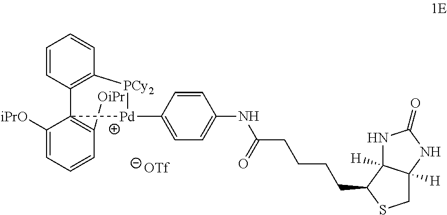

Following the general procedure, a mixture containing aryl trifluoromethanesulfonate Si (100.0 mg, 0.21 mmol, Note: 1 equiv was used), RuPhos (109.8 mg, 0.24 mmol), and (COD)Pd(CH₂SiMe₃)₂ (83.2 mg, 0.21 mmol) was stirred in THF (1.5 mL) at rt for 16 h. General work up afforded 1E as a light orange solid (179.0 mg, 80%).

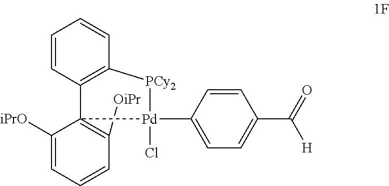

Following the general procedure, a mixture containing 4-chlorobenzaldehyde (39.7 mg, 0.28 mmol), RuPhos (131.9 mg, 0.28 mmol), and (COD)Pd(CH₂SiMe₃)₂ (100.0 mg, 0.26 mmol) was stirred in cyclohexane (1.5 mL) at rt for 16 h. General work up afforded 1F as a white solid (166.0 mg, 91%).

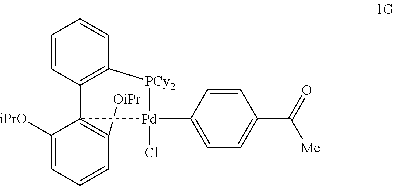

Following the general procedure, a mixture containing 4-chloroacetophenone (36.7 L, 0.28 mmol), RuPhos (131.9 mg, 0.28 mmol), and (COD)Pd(CH₂SiMe₃)₂ (100.0 mg, 0.26 mmol) was stirred in cyclohexane (1.5 mL) at rt for 16 h. General work up afforded 1G as a white solid (187.1 mg, 80%).

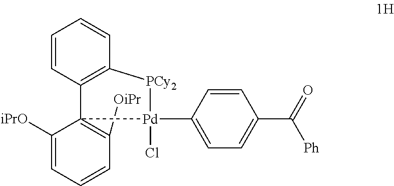

Following the general procedure, a mixture containing 4-chlorobenzophenone (61.2 mg, 0.28 mmol), RuPhos (131.9 mg, 0.28 mmol), and (COD)Pd(CH₂SiMe₃)₂ (100.0 mg, 0.26 mmol) was stirred in cyclohexane (1.5 mL) at rt for 16 h. General work up afforded 1H as a white solid (170.3 mg, 84%).

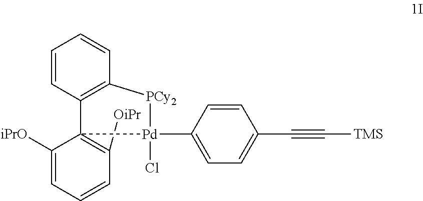

Following the general procedure, a mixture containing (4-chlorophenylethynyl)trimethylsilane (71.6 mg, 0.34 mmol), RuPhos (131.9 mg, 0.28 mmol), and (COD)Pd(CH₂SiMe₃)₂ (100.0 mg, 0.26 mmol) was stirred in cyclohexane (1.5 mL) at rt for 16 h. General work up afforded 1I as a white solid (157.7 mg, 78%).

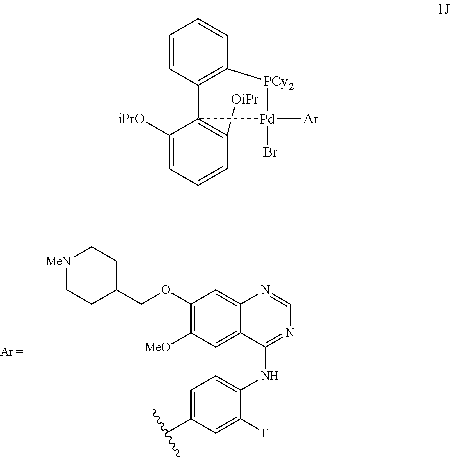

Following the general procedure, a mixture containing Vandetanib (61.7 mg, 0.13 mmol, Note: 1.01 equiv was used), RuPhos (66.0 mg, 0.14 mmol), and (COD)Pd(CH₂SiMe₃)₂ (50.0 mg, 0.13 mmol) was stirred in THF (1.5 mL) at rt for 16 h.

General work up afforded 1J as an off-white solid (119.0 mg, 88%).

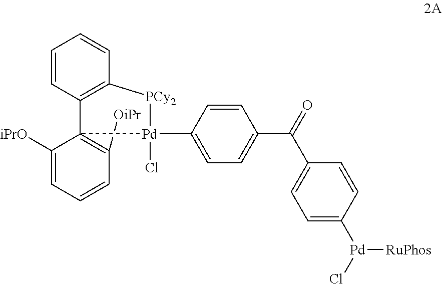

Following a slightly modified general procedure, a mixture of 4,4′-dichlorobenzophenone (30.0 mg, 0.12 mmol, 1 equiv), RuPhos (139.4 mg, 0.30 mmol, 2.5 equiv), and (COD)Pd(CH₂SiMe₃)₂ (116.2 mg, 0.30 mmol, 2.5 equiv) was stirred in cyclohexane (1.2 mL) at rt for 16 h. General work up afforded 2A as a beige solid (146.8 mg, 88%).

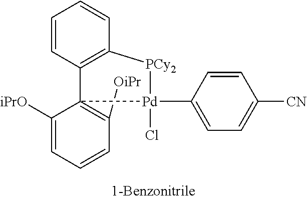

Following the general procedure, a mixture of 4-chlorobenzonitrile (42.4 mg, 0.31 mmol), RuPhos (144.0 mg, 0.31 mmol), and (COD)Pd(CH₂SiMe₃)₂ (100.0 mg, 0.26 mmol) was stirred in cyclohexane (1.5 mL) at rt for 16 h. General work up afforded 1-Benzonitrile as a white solid (186.4 mg, 99%).

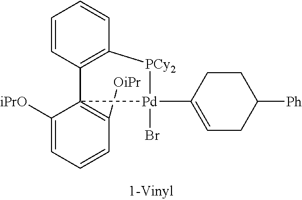

Following the general procedure, a mixture containing 4-bromo-1,2,3,6-tetrahydro-1,1′-biphenyl (30.0 mg, 0.127 mmol), RuPhos (59.0 mg, 0.127 mmol), and (COD)Pd(CH₂SiMe₃)₂ (44.7 mg, 0.115 mmol) was stirred in cyclohexane (0.75 mL) at rt for 16 h. General work up afforded 1-Vinyl as a yellow solid (80.0 mg, 86%).

### Example 14—Arylation Reaction Conditions

Many of the exemplified cysteine conjugation reactions operate at nearly neutral to slightly basic pH values. Further evaluation of the reaction conditions using palladium reagents revealed quantitative conversion of the starting peptide to the corresponding S-aryl cysteine conjugate within a broad pH range (5.5-8.5) using common organic cosolvents (5% of DMF, DMSO, CH₃CN) in various buffers. Remarkably, even in 0.1% TFA solution (pH 2.0) the reaction yielded 59% of the S-arylated product after 7 hours. The process was also compatible with the protein disulfide reducing agent tris(2-carboxyethyl)phosphine (TCEP) that has been shown to hamper bioconjugations by reacting with maleimide and α-haloacyl groups

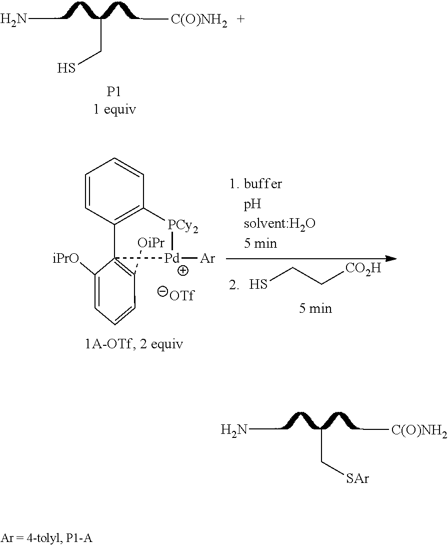

Reaction condition evaluation.^{a}

| | | Peptide | | | |
|---|---|---|---|---|---|
| Entry | Buffer | Conc. | pH | Solvent | Product |
| 1 | 100 mM Tris | 1 mM | 8.5 | H₂O:CH₃CN (2:1) | 93% |
| 2 | 100 mM Tris | 100 μM | 8.5 | H₂O:CH₃CN (95:5) | 85% |
| 3 | 100 mM Tris | 10 μM | 8.5 | H₂O:CH₃CN (95:5) | 100% |
| 4 | 100 mM Tris | 10 μM | 8 | H₂O:CH₃CN (95:5) | 100% |
| 5 | 100 mM Tris | 10 μM | 7.5 | H₂O:CH₃CN (95:5) | 100% |
| 6 | 100 mM HEPES | 10 μM | 7.5 | H₂O:CH₃CN (95:5) | 100% |
| 7 | 100 mM MOPS | 10 μM | 7.5 | H₂O:CH₃CN (95:5) | 100% |
| 8 | 100 mM | 10 μM | 7.5 | H₂O:CH₃CN (95:5) | 100% |
| | Na₂HPO₄/ | | | | |
| | NaH₂PO₄ | | | | |
| 9 | 25 mM Tris | 10 μM | 7.5 | H₂O:CH₃CN (95:5) | 93% |
| 10 | 100 mM Tris | 10 μM | 7 | H₂O:CH₃CN (95:5) | 84% |
| 11 | 100 mM MOPS | 10 μM | 6.5 | H₂O:CH₃CN (95:5) | 100% |
| 12 | 100 mM MES | 10 μM | 5.5 | H₂O:CH₃CN (95:5) | 95% |
| 13^{b} | 100 mM MES | 10 μM | 5.5 | H₂O:CH₃CN (95:5) | 100% |
| 14 | 0.1% TFA | 10 μM | 2.0 | H₂O:CH₃CN (95:5) | 18% |
| 15^{c} | 0.1% TFA | 10 μM | 2.0 | H₂O:CH₃CN (95:5) | 59% |
| 16 | 100 mM Tris | 10 μM | 7.5 | H₂O:DMF (95:5) | 100% |
| 17 | 100 mM Tris | 10 μM | 7.5 | H₂O:DMSO (95:5) | 100% |
| 18^{d} | 100 mM Tris | 10 μM | 7.5 | H₂O:CH₃CN (95:5) | 100% |
| 19^{e} | 100 mM Tris | 1 mM | 8.5 | H₂O:CH₃CN (2:1) | 0% |

^{a}Optimal conditions used for further substrate scope evaluation are highlighted in grey;
^{b}Reaction time: 10 min;
^{c}Reaction time: 7 h 20 min;
^{d}Reaction performed in the presence of TCEP (20 μM);
^{e}Peptide P1-Ser was used as the control.

| | | Calculated | Observed |
|---|---|---|---|
| Peptide | Sequence^{a} | mass | mass |
| P1 | NH₂-RSNFYLGCAGLAHDKAT- | 1821.89 | 1821.89 |
| | CONH₂ | | |
| P1-Ser | NH₂-RSNFYLGSAGLAHDKAT- | 1805.92 | 1805.92 |
| | CONH₂ | | |
| P2 | NH₂-RSNFFLGCAGA-CONH₂ | 1140.55 | 1140.55 |
| P3 | NH₂-IKFTNCGLLCYESKR- | 1772.91 | 1772.91 |
| | CONH₂ | | |

### Example 15—Exemplary Arylation Reactions

The palladium mediated conjugation is fast, with complete product formation occurring within 15 seconds at 4° C. The reaction rate was estimated by competition experiments against the commonly used N-methyl maleimide cysteine ligation. (Gorin, G., et al. Arch. Biochem. Biophys. 115, 593-597 (1966)). At pH 7.5, the rate of the palladium-mediated reaction was comparable to that of the maleimide ligation, where 70% of the products resulted from the reaction with palladium-tolyl complex (1A-OTf). Notably, the palladium-mediated conjugation outperformed the maleimide ligation at pH 5.5, at which only the arylated product was formed.

The optimized conditions (0.1 M Tris buffer, 5% CH₃CN, pH 7.5, room temperature) were used for further evaluation of the substrate scope (General Arylation Procedure A). Palladium complexes containing chloride, bromide and iodide counterions were all found to produce the desired product (1A-CI, 1A-Br, and 1A-I). This method can be used to functionalize unprotected peptides with a variety of important groups including fluorescent tags (1C, 1D), affinity labels (1E), bioconjugation handles (aldehyde 1F, ketone 1G, and alkyne 1H), photochemical crosslinkers (1I), as well as complex drug molecules (1J). Importantly, the palladium(II) complexes are stable under ambient conditions, and can be stored in closed vials under air at 4° C. for over four months. The “aged” reagents still exhibited reactivity comparable to the freshly made complexes.

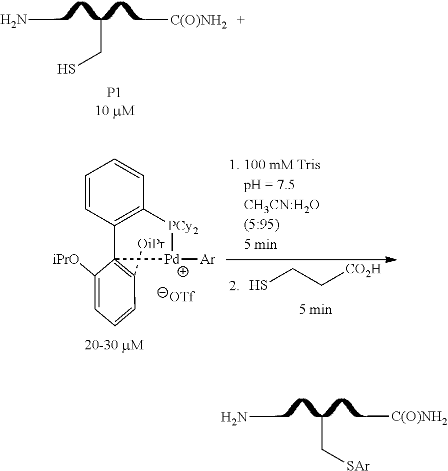

General Arylation Procedure A.

Peptide P1 (4 μL, 150 μM in water), H₂O (47 μL), organic solvent (1 μL), and the buffer (6 μL, 1 M) were combined in a 0.6 mL plastic Eppendorf tube and the resulting solution was mixed by vortexing for 10 s. A stock solution of the palladium complex (2 μL, 600 μM) in organic solvent was added in one portion, the reaction tube was vortexed to ensure proper reagent mixing and left at room temperature for 5 min. The reaction was quenched by the addition of 3-mercaptopropionic acid (6.3 μL, 0.05 μL/mL solution in water, 3 equiv to the palladium complex). After an additional 5 min, a solvent mixture of (e.g., 50% A:50% B (v/v, 60 μL)) was added to the Eppendorf and the reaction mixture was analyzed by LC-MS.

Final concentrations of the reaction before quenching: peptide P1—10 μM, Pd-complex—20 μM, Buffer—100 mM; organic solvent: H₂O=5:95.

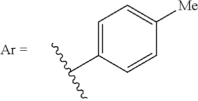 [SMILES: *c1ccc(C)cc1]

The arylated peptide P1-A was synthesized according to general procedure A. Final conditions before quenching: peptide—10 μM, 1A-OTf—20 μM, 0.1 M Tris (pH 7.5), CH₃CN:H₂O=5:95.

Me Et

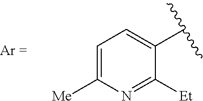 [SMILES: *c1ccc(C)nc1CC]

The arylated peptide P1-B was synthesized according to general procedure A. Final conditions before quenching: peptide—10 μM, 1B—20 μM, 0.1 M Tris (pH 7.5), CH₃CN:H₂O=5:95.

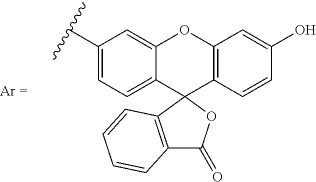 [SMILES: *c1ccc2c(c1)Oc1cc(O)ccc1C21OC(=O)c2ccccc21]

The arylated peptide P1-C was synthesized according to general procedure A. The reaction was quenched by the addition of 3-mercaptopropionic acid (12.5 μL, 0.05 μL/mL solution in water, 2 equiv to 1C). Final conditions before quenching: peptide—10 μM, 1C—30 μM, 0.1 M Tris (pH 7.5), CH₃CN:H₂O=5:95.

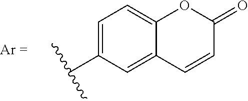 [SMILES: *c1ccc2oc(=O)ccc2c1]

The arylated peptide P1-D was synthesized according to general procedure A. The reaction was quenched by the addition of 3-mercaptopropionic acid (6.3 μL, 0.05 μL/mL solution in water, 2 equiv to 1D). Final conditions before quenching: peptide—10 μM, 1D—30 μM, 0.1 M Tris (pH 7.5), CH₃CN:H₂O=5:95.

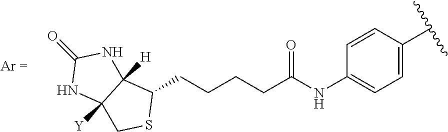

The arylated peptide P1-E was synthesized according to general procedure A. Final conditions before quenching: peptide—10 μM, 1E—20 μM, 0.1 M Tris (pH 7.5), CH₃CN:H₂O=5:95.

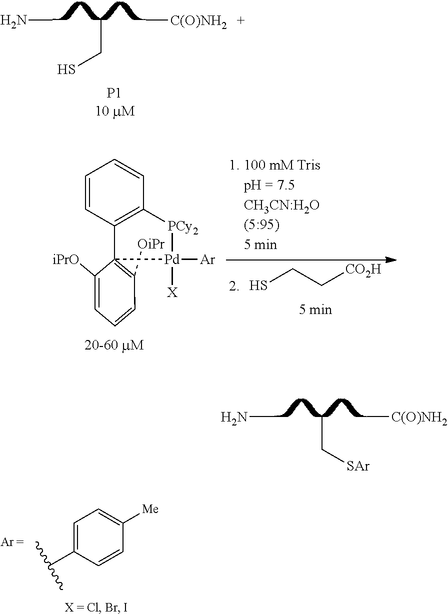

The arylated peptide P1-A was synthesized according to general procedure A. Final conditions before quenching: peptide—10 μM, 1A-X (X=Cl, Br, I)—20 μM, 0.1 M Tris (pH 7.5), CH₃CN:H₂O=5:95.

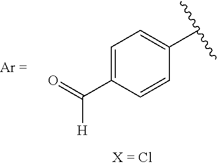 [SMILES: *c1ccc(C=O)cc1]

The arylated peptide P1-F was synthesized according to general procedure A. Final conditions before quenching: peptide—10 μM, 1F—20 μM, 0.1 M Tris (pH 7.5), CH₃CN:H₂O=5:95.

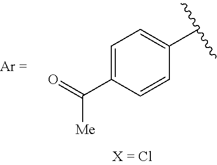 [SMILES: *c1ccc(C(C)=O)cc1]

The arylated peptide P1-G was synthesized according to general procedure A. Final conditions before quenching: peptide—10 μM, 1G—20 μM, 0.1 M Tris (pH 7.5), CH₃CN:H₂O=5:95.

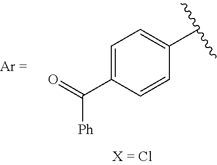 [SMILES: *c1ccc(C(=O)c2ccccc2)cc1]

The arylated peptide P1-H was synthesized according to general procedure A. Final conditions before quenching: peptide—10 μM, 1H—20 μM, 0.1 M Tris (pH 7.5), CH₃CN:H₂O=5:95.

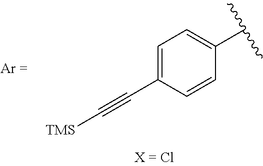 [SMILES: *c1ccc(C#C[Si](C)(C)C)cc1]

The arylated peptide P1-I was synthesized according to general procedure A. The reaction was quenched by the addition of 3-mercaptopropionic acid (6.3 μL, 0.05 μL/mL solution in water, 1 equiv to 1I). Final conditions before quenching: peptide—10 μM, 1I—60 μM, 0.1 M Tris (pH 7.5), CH₃CN:H₂O=5:95.

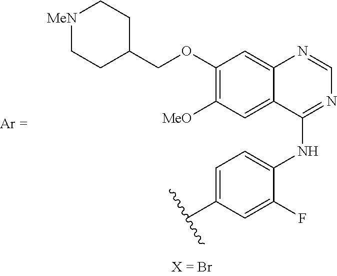 [SMILES: *c1ccc(Nc2ncnc3cc(OCC4CCN(C)CC4)c(OC)cc23)c(F)c1]

The arylated peptide P1-J was synthesized according to general procedure A. Final conditions before quenching: peptide—10 μM, 1J—20 μM, 0.1 M Tris (pH 7.5), CH₃CN:H₂O=5:95.

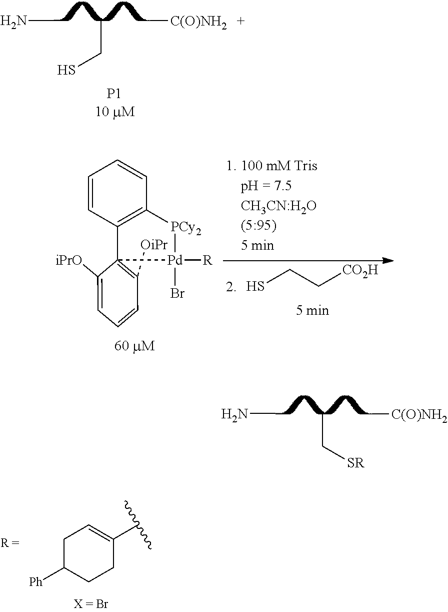

The vinylated peptide P1-Vinyl was synthesized according to general procedure A. The reaction was quenched by the addition of 3-mercaptopropionic acid (6.3 μL, 0.05 μL/mL solution in water, 1.5 equiv to 1-Vinyl). Final conditions before quenching: peptide—10 μM, 1-Vinyl—40 μM, 0.1 M Tris (pH 7.5), CH₃CN:H₂O=5:95.

### Example 16—Stability Evaluation of the Arylated Peptides

The stability of the arylated peptides was compared to that of conjugates formed from reactions with reagents including N-ethyl maleimide, 2-bromoacetamide, and benzyl bromide. The S-arylated peptide was shown to be stable toward acids, bases, and external thiol nucleophiles. In contrast, the corresponding acetamide derivative was unstable under acidic and basic conditions and the maleimide conjugate decomposed in the presence of base and exogenous thiol. Finally, comparable stability of both aryl and benzyl conjugates to treatment with the periodic acid oxidant at 37° C. was observed.

### Stability Evaluation in the Presence of Base, Acid or an External Thiol Nucleophile

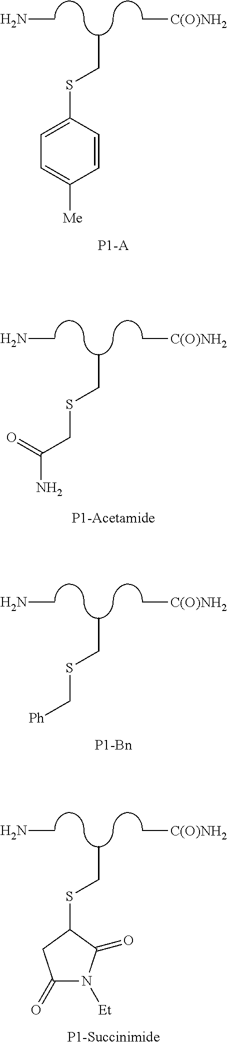

Peptide P1 conjugates were pre-dissolved in water in plastic Eppendorfs to afford the 1.11 mM stock solutions used in the stability evaluation experiments. For each experiment, the corresponding cysteine conjugate (1.11 mM; 18 μL) and stability test reagent (2 μL, 50 mM in H₂O or 50 mM in 1M Tris, pH 7.4) were combined in a plastic Eppendorf and left at rt for 2 days, followed by 4 days at 37° C. After this time, individual reactions were quenched with a solution of 50% A:50% B (v/v, 200 μL) and the resulting samples were analyzed by LC-MS.

### Basic Conditions

Stability test reagent: K₂CO₃ (2 μL, 50 mM in H₂O);
Final conditions before quenching: 1 mM peptide, 5 mM K₂CO₃; 2 d at rt, then 4 d at 37° C.

### Acidic Conditions

Stability test reagent: HCl (2 μL, 1 M in H₂O);
Final conditions before quenching: 1 mM peptide, 0.1 M HCl; 2 d at rt, then 4 d at 37° C.

### Presence of External Thiol Nucleophiles: GSH

Stability test reagent: Glutathione (2 μL, 50 mM in 1 M Tris; pH 7.4);
Final conditions before quenching: 1 mM peptide, 5 mM GSH, 0.1M Tris, pH 7.4; 2 d at rt, then 4 d at 37° C.

TABLE
Stability of the cysteine conjugates under basic and acidic conditions,
as well as in the presence of external thiol nucleophiles.

| | 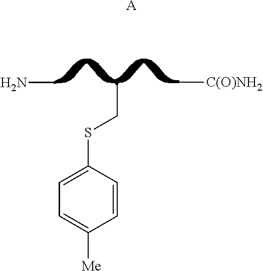 | 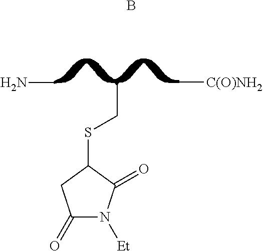 |
|---|---|---|
| | % remaining peptide | |
| base | 83% | 0% |
| acid | 83% | 84% |
| GSH | 90% | 37% |
| | 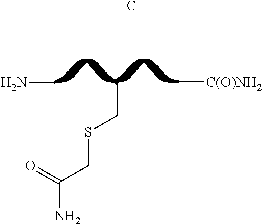 | 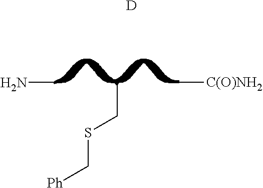 |
| | % remaining peptide | |
| base | 66% | 84% |
| acid | 63% | 85% |
| GSH | 85% | 88% |

### Stability of Cysteine Conjugates Toward Oxidation

Additional tuning of the electronic properties of the aromatic ring of the arylated peptide by installing a para-electron withdrawing cyano-group could be achieved. This modification significantly decreased the amount of oxidation producing the most stable peptides across all the evaluated conjugates. Notably, installing the para cyano-group in the benzyl conjugates did not have any effect toward oxidation.

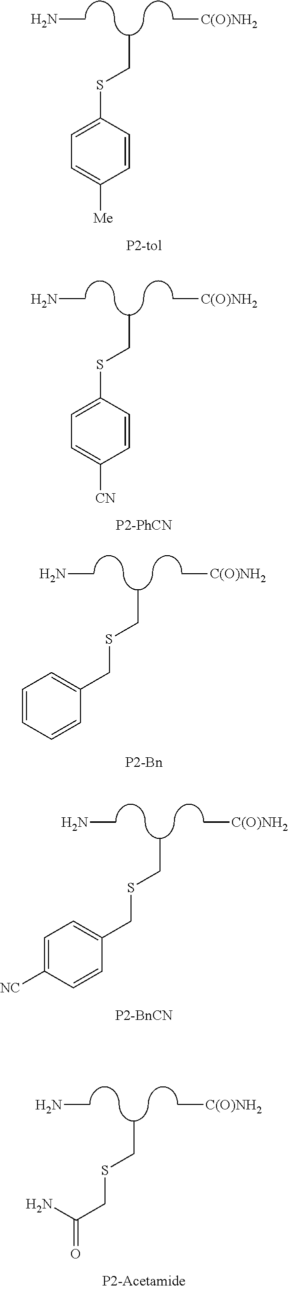

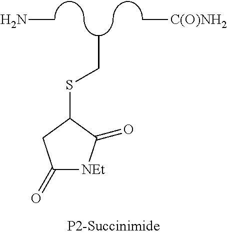

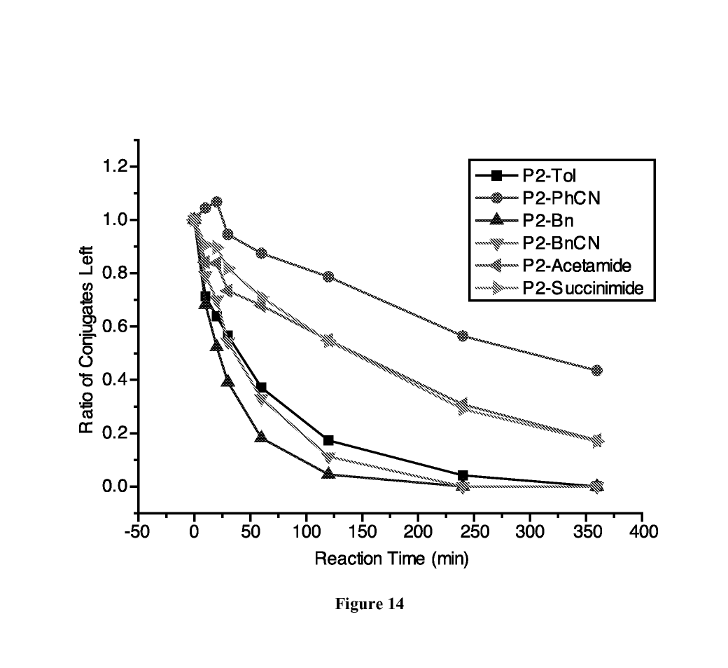

Peptide P2 conjugates were pre-dissolved in water in plastic Eppendorfs to afford the 111.1 μM stock solutions used in the oxidation stability evaluation experiments. The corresponding cysteine conjugates (18 μL, 111.1 μM in H₂O) and H₅IO₆ (2 μL, 4 mM in H₂O) were then combined in a plastic Eppendorf, mixed using a vortexer and transferred into a pre-heated water bath at 37° C. Individual reactions were quenched with Na₂SO₃ (20 L, 4 mM in H₂O) after 10 min, 30 min, 1 h, 2 h, 4 h, and 6 h, and the resulting mixtures were kept at rt for an additional 10 min. Subsequently, a solution of 50% A:50% B (v/v, 160 μL) was added and the resulting samples were analyzed by LC-MS (FIG. 14). Final conditions before quenching: 100 μM peptide, 400 μM H₅IO₆, 37° C.

### Example 17—Protein Modification

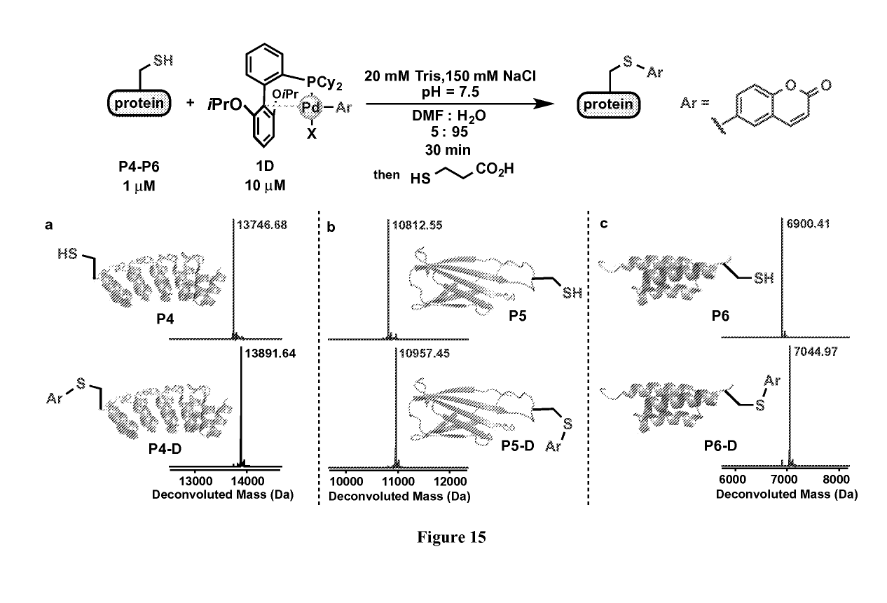

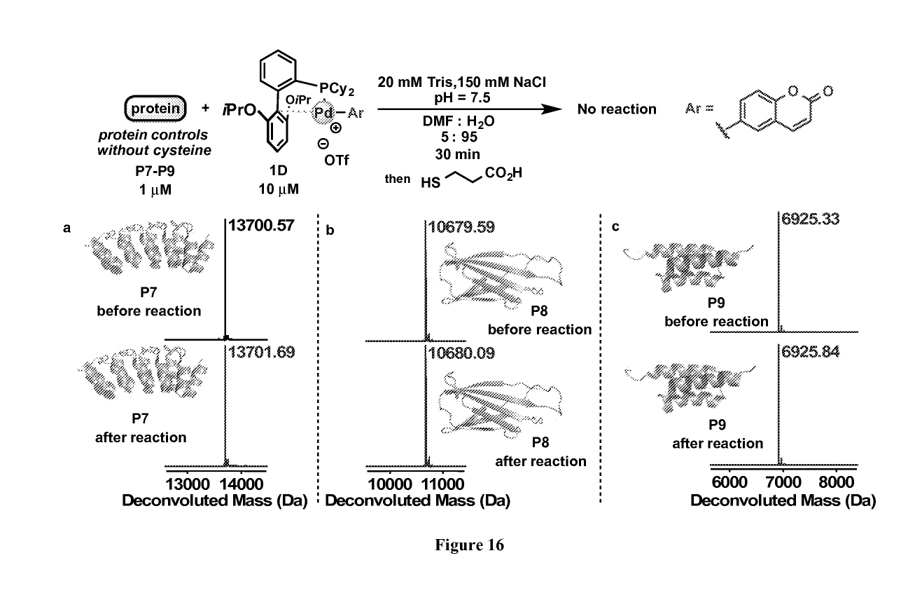

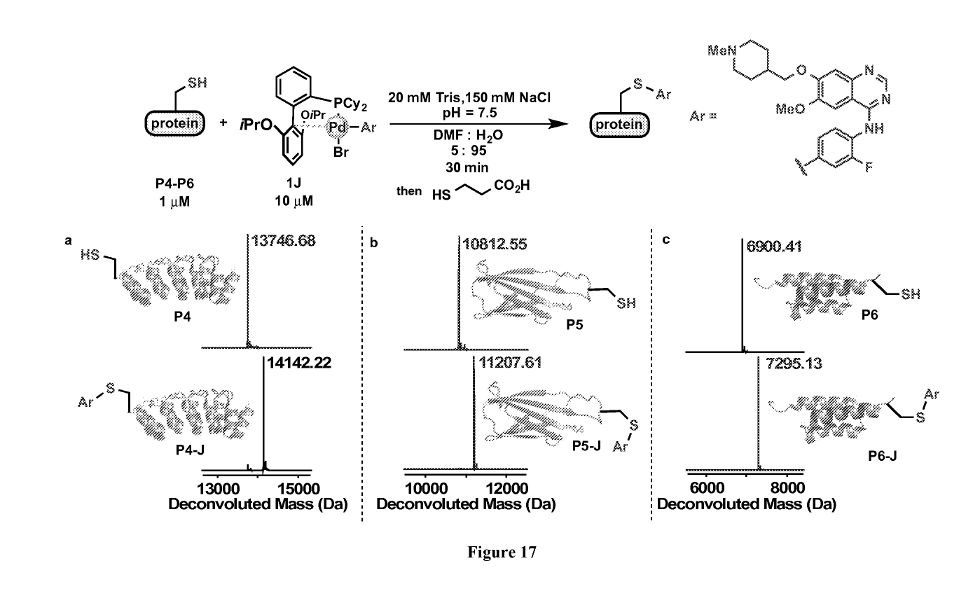

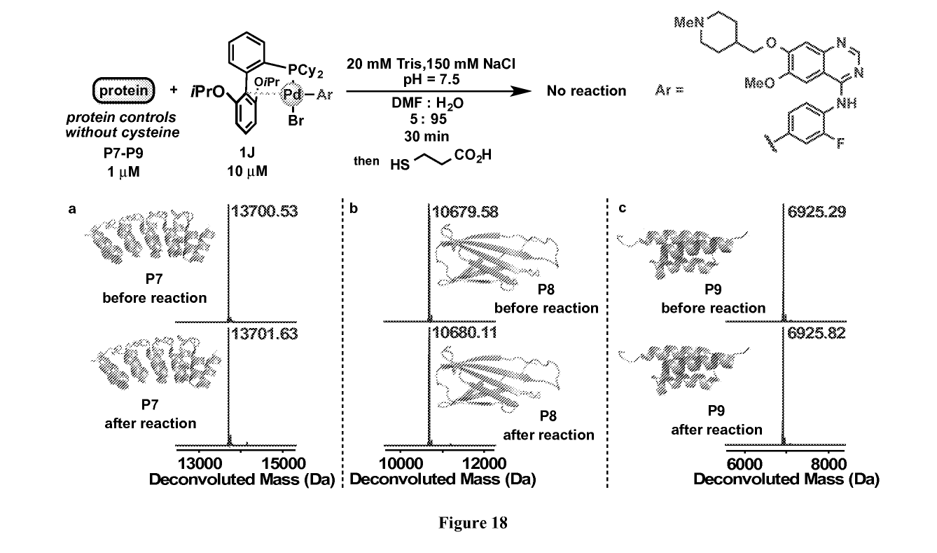

This reaction was explored with proteins. Three antibody mimetic proteins (P4-P6) were expressed that contained a cysteine at structurally distinct positions including the N-terminus, C-terminus, and a loop. The same proteins without cysteine were used as controls to confirm the selectivity of the reaction (P7-P9). All three proteins (P4-P6) were quantitatively tagged with either coumarin (FIG. 15) or a drug molecule (FIG. 17) within 30 minutes at 1 μM protein concentration. No arylated product was generated for proteins lacking a cysteine (FIGS. 16 and 18). The fast kinetics and high efficiency of the reactions at low micromolar protein concentrations are in contrast to reported bioconjugation methods using organometallic reagents, where longer reaction times were needed and generally lower conversions were observed (Kung, K. K.-Y. et al. Chem. Commun. 50, 11899-11902 (2014)). The modified proteins can be readily separated from the remaining palladium species, ligands, and other small molecules using standard desalting techniques.

Protein Labeling

To a solution of protein (500 pmoles) in 475 μL of 20 mM Tris and 150 mM NaCl buffer (pH 7.5) was added palladium-coumarin complex 1D or palladium-drug complex 1J (25 μL, 200 μM) in DMF. The solution was pipetted up and down 20 times to ensure proper reagent mixing. The reaction mixture was left at room temperature for 30 min. After this time, the reaction was quenched by the addition of 3-mercaptopropionic acid (25 μL, 2 mM) dissolved in 20 mM Tris and 150 mM NaCl buffer (pH 7.5). After an additional 5 min at rt, 500 μL of 1:1 CH₃CN/H₂O (v/v) containing 0.2% TFA was added and the resulting mixture was analyzed by LC-MS.

Protein P4: DARPin-Cys
Calculated Mass: 13747.3 Da
Sequence:
GGCGGSDLGKKLLEAARAGQDDEVRILMANGADVNAYDDNGVTPLHLA
AFLGHLEIVEVLLKYGADVNAADSWGTTPLHLAATWGHLEIVEVLLKHGA
DVNAQDKFGKTAFDISIDNGNEDLAEILQKLN
Protein P7: DARPin
Calculated Mass: 13701.3 Da
Sequence:
GGGGGSDLGKKLLEAARAGQDDEVRILMANGADVNAYDDNGVTPLHLA
AFLGHLEIVEVLLKYGADVNAADSWGTTPLHLAATWGHLEIVEVLLKHGA
DVNAQDKFGKTAFDISIDNGNEDLAEILQKLN
Protein P5: 10FN3-Cys
Calculated Mass: 10813.1 Da
Sequence:
SVSDVPRDLEVVAATPTSLLISWDAPAVTVRYYRITYGETGGNSPVQEF
TVPGSKSTATISGLKPGVDYTITVYAVTLPSTCGASSKPISINYRTEID
KPSQ
Protein P8: 10FN3
Calculated Mass: 10679.9 Da
Sequence:
VSDVPRDLEVVAATPTSLLISWDAPAVTVRYYRITYGETGGNSPVQEFTV
PGSKSTATISGLKPGVDYTITVYAVTLPSTGGASSKPISINYRTEIDKP
SQ
Protein P6: Affibody-Cys
Calculated Mass: 6900.6
Sequence:
GGGGGVDNKFNKEQQNAFYEILHLPNLNEEQRNAFIQSLKDDPSQSANLL
AEAKKLNDACAPK
Protein P9: Affibody
Calculated Mass: 6925.6 Da
Sequence:
GGGGGVDNKFNKEQQNAFYEILHLPNLNEEQRNAFIQSLKDDPSQSANLL
AEAKKLNDAQAPK

### Example 18—Reactivity of Haloarylated Products

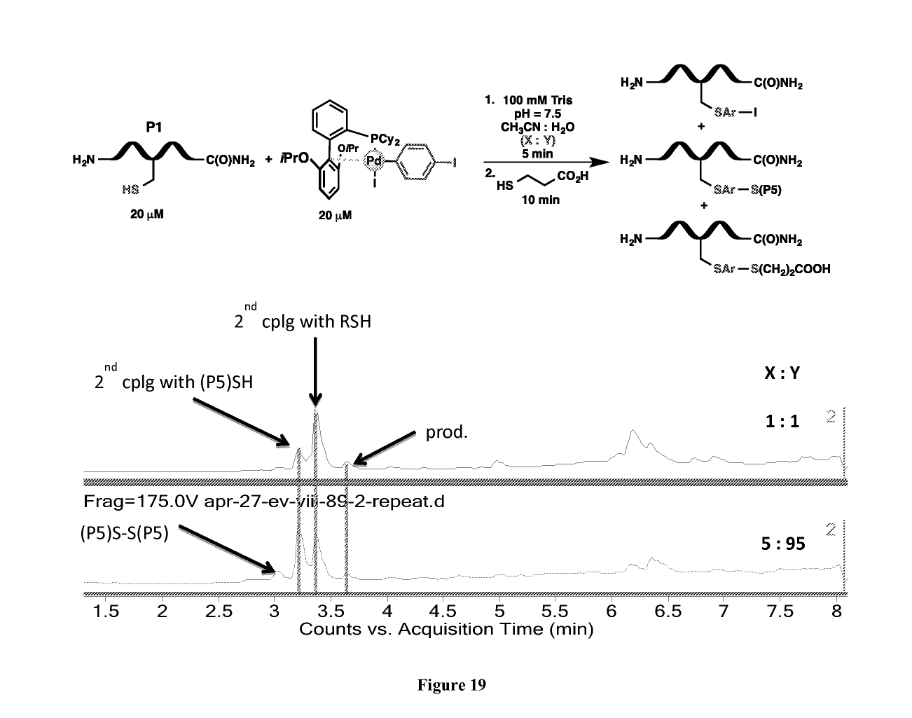

Haloarylated peptides (i.e., containing an aryl-halide bond) can undergo further cross-coupling reaction with external thiols to generate arylated peptides with additional complexity. As demonstrated in FIG. 19, the products of the peptide arylation reaction have undergone reaction with other thiol-containing peptides or even with the thiol-containing quenching agent.

### Example 19—Stapled Peptides

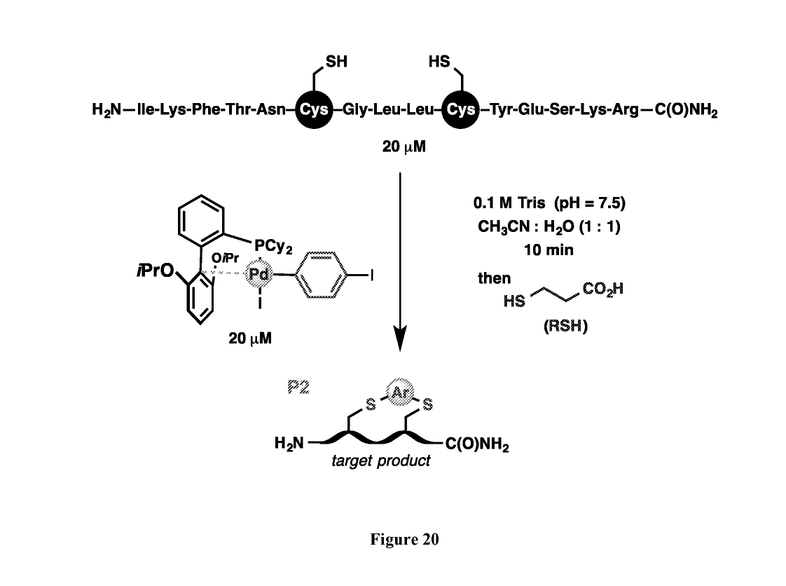

The stapled peptides discussed herein can also be generated by an alternative non-symmetric process. A monopalladium haloarylation reagent (i.e., a reagent containing an aryl halide bond) has undergone reaction with a cysteine-containing peptide. After this first cross coupling reaction step, a secondary cross coupling reaction with the catalyst at a second cysteine residue in the peptide yielded the target stapled peptide product (FIG. 20).

### Example 20—Biomolecule Arylation with Precatalysts

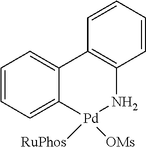

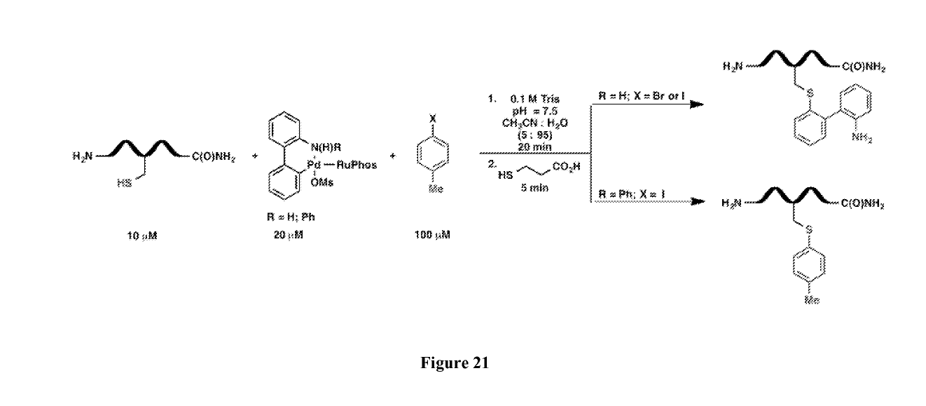

Air-stable Ph-mesylate palladium precatalysts (e.g., 2-amino biphenyl Pd species such as the second generation Buchwald catalyst) can also be used as catalysts for the biomolecule arylation reaction. When used in conjunction with an aryl halide reagent, these precatalysts generated arylated peptide products (FIG. 21).

### INCORPORATION BY REFERENCE

All of the U.S. patents and U.S. patent application publications cited herein are hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method of functionalizing a thiol or selenol, wherein said method is represented by Scheme 1: 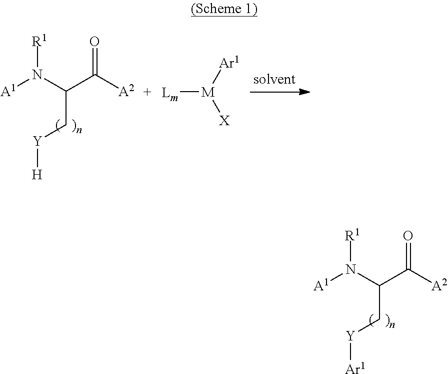
wherein:
A¹ is H, an amine protecting group, alkyl, arylalkyl, acyl, aryl, alkoxycarbonyl, aryloxycarbonyl, a natural or unnatural amino acid, a plurality of natural amino acids or unnatural amino acids, a peptide, an oligopeptide, a polypeptide, a protein, an antibody, or an antibody fragment;
A² is NH₂, NH(amide protecting group), N(amide protecting group), OH, O(carboxylate protecting group), a natural or unnatural amino acid, a plurality of natural amino acids or unnatural amino acids, a peptide, an oligopeptide, a polypeptide, a protein, an antibody, or an antibody fragment;
Y is S or Se;
R¹ is H, alkyl, arylalkyl, acyl, aryl, alkoxycarbonyl, aryloxycarbonyl, a natural or unnatural amino acid, a plurality of natural amino acids or unnatural amino acids, a peptide, an oligopeptide, a polypeptide, a protein, an antibody, or an antibody fragment;
M is Ni, Pd, Pt, Cu, or Au;
Ar¹ is optionally substituted aryl, heteroaryl, alkenyl, or cycloalkenyl;
X is a halide, triflate, tetrafluoroborate, tetraarylborate, hexafluoroantimonate, bis(alkylsulfonyl)amide, tetrafluorophosphate, hexafluorophosphate, alkylsulfonate, haloalkylsulfonate, arylsulfonate, perchlorate, bis(fluoroalkylsulfonyl)amide, bis(arylsulfonyl)amide, (fluoroalkylsulfonyl)(fluoroalkyl-carbonyl)amide, nitrate, nitrite, sulfate, hydrogensulfate, alkyl sulfate, aryl sulfate, carbonate, bicarbonate, carboxylate, phosphate, hydrogen phosphate, dihydrogen phosphate, phosphinate, or hypochlorite;
L is independently for each occurrence a trialkylphosphine, a triarylphosphine, a dialkylarylphosphine, an alkyldiarylphosphine, an (alkenyl)(alkyl)(aryl)phosphine, an alkenyldiarylphosphine, an alkenyldialkylphosphine, a phosphine oxide, a bis(phosphine), a phosphoramide, a triarylphosphonate, an N-heterocyclic carbene, an optionally substituted phenanthroline, an optionally substituted iminopyridine, an optionally substituted 2,2′-bipyridine, an optionally substituted diimine, an optionally substituted triazolylpyridine, or an optionally substituted pyrazolyl pyridine;
n is an integer from 1-5;
m is 1 or 2; and
solvent is a polar protic solvent, a polar aprotic solvent, or a non-polar solvent.

2. The method of claim 1, wherein L is selected from the group consisting of PPh₃, Ph₂P—CH₃, PhP(CH₃)₂, P(o-tol)₃, PCy₃, P(tBu)₃, BINAP, dppb, dppe, dppf, dppp, 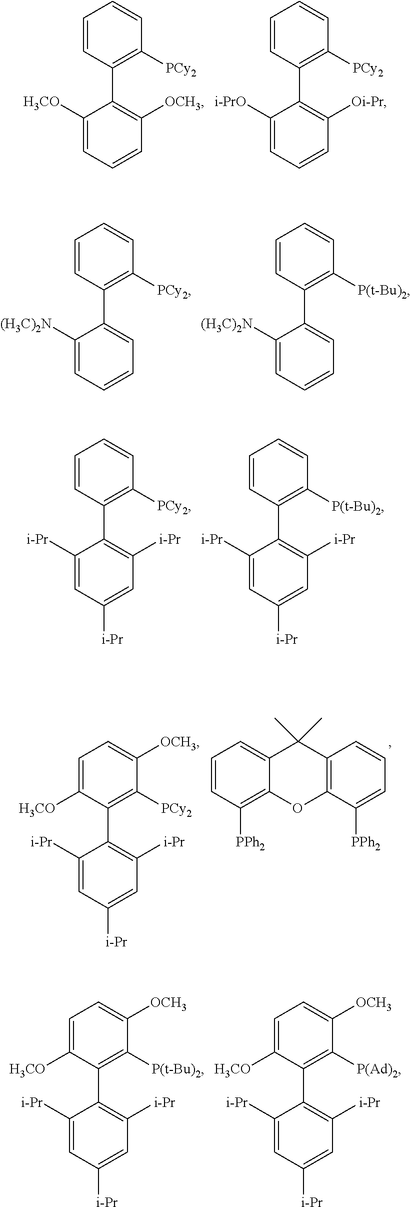 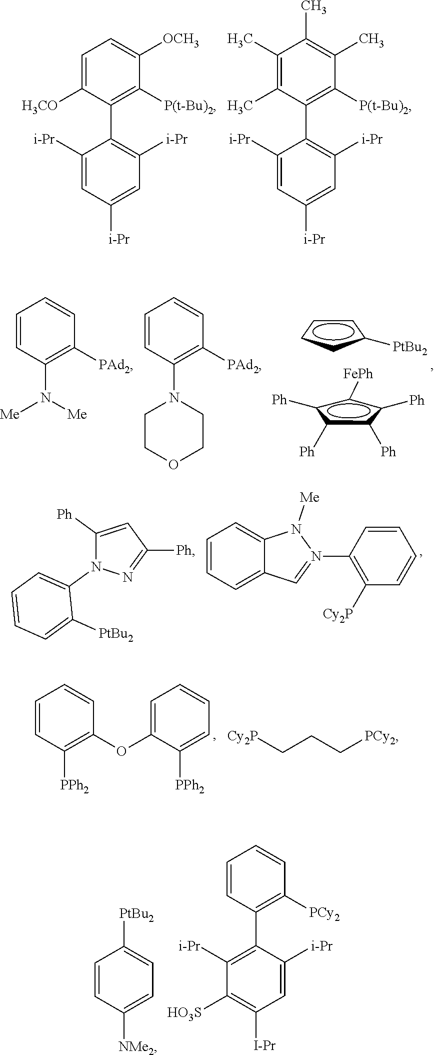
or its salt, 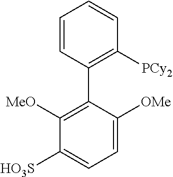
or its salt, 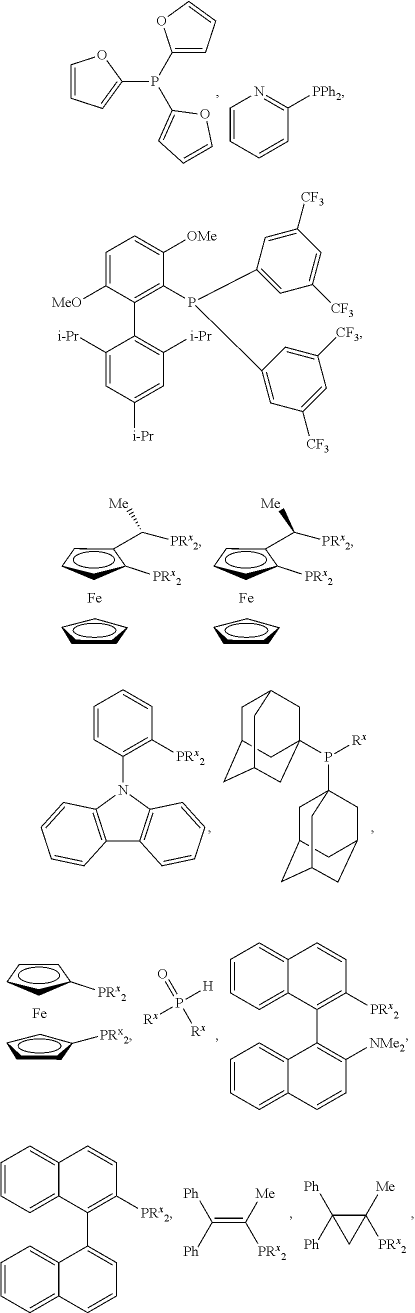 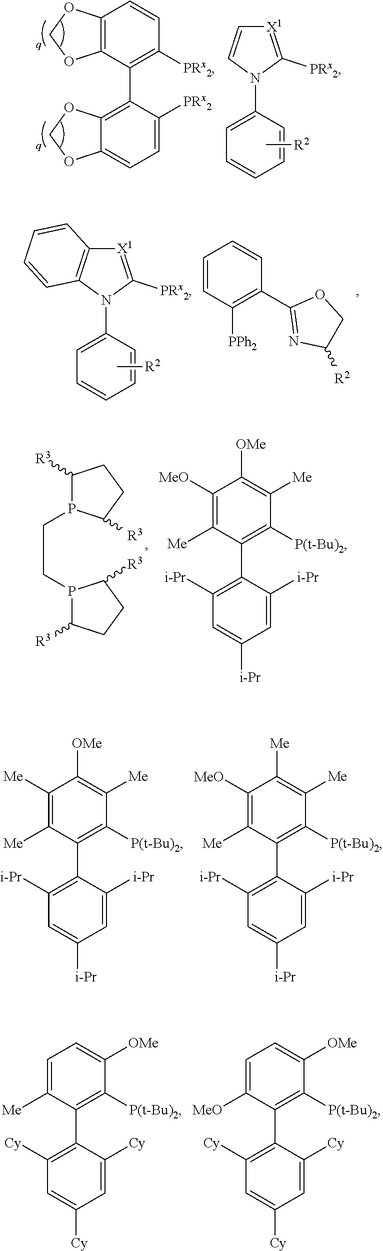 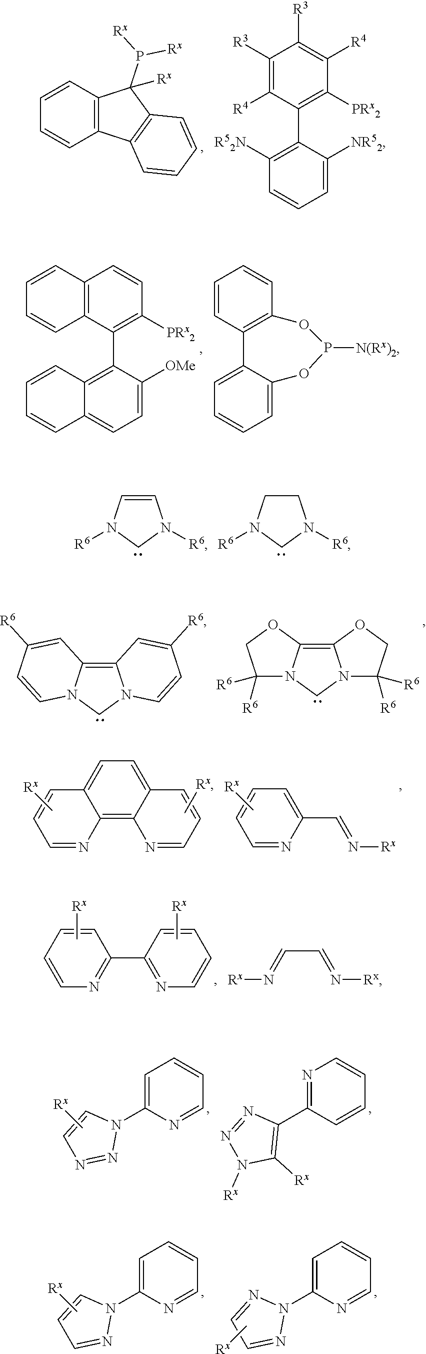 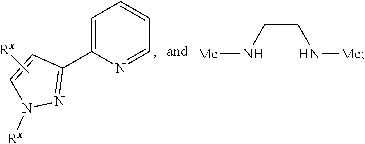
R^{x} is independently for each occurrence alkyl, aralkyl, cycloalkyl, or aryl;
X¹ is CH or N;
R² is H or alkyl;
R³ is H or alkyl;
R⁴ is H, alkoxy, or alkyl;
R⁵ is alkyl or aryl;
R⁶ is alkyl or aryl; and
q is 1, 2, 3, or 4.

3. (canceled)

4. The method of claim 2, wherein M is Pd or Ni.

5. The method of claim 2, wherein M is Pd; and L is 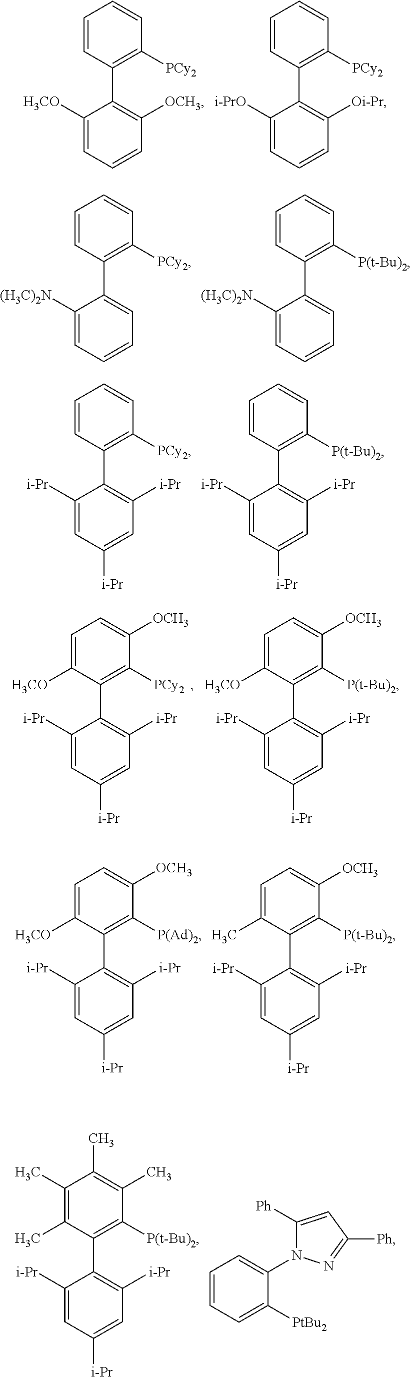 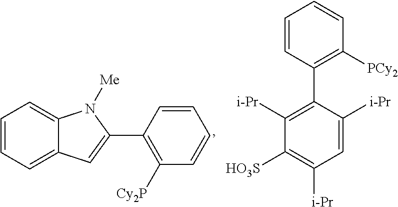
or its salt, 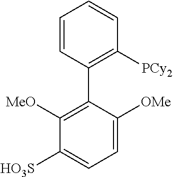
or its salt,

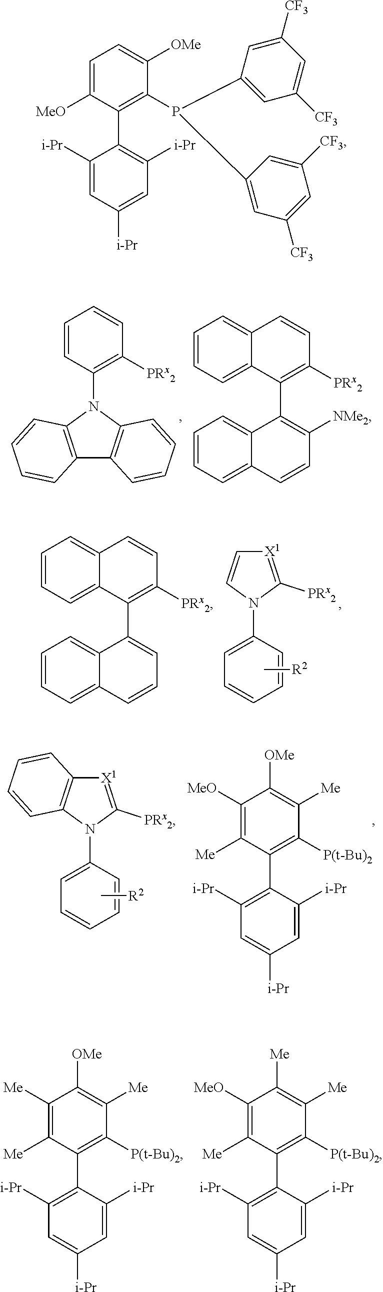

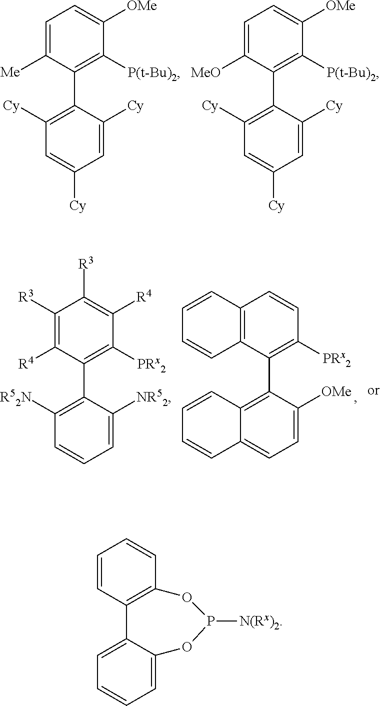

6. (canceled)

7. (canceled)

8. The method of claim 2, wherein M is Ni; and L is BINAP, dppb, dppe, dppf, dppp,

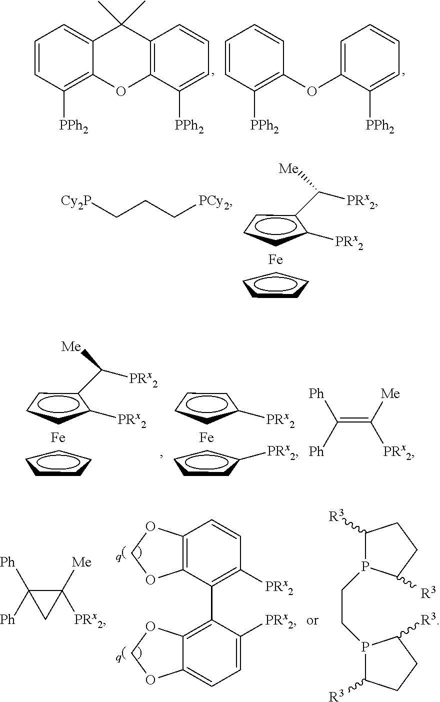

9-11. (canceled)

12. The method of claim 1, wherein X is halide or triflate.

13. The method of claim 1, wherein
Ar¹ is (C₆-C₁₀)carbocyclic aryl, (C₃-C₁₂)heteroaryl, (C₃-C₁₄)polycyclic aryl, or alkenyl; and Ar¹ is optionally substituted by one or more substituents independently selected from the group consisting of halide, acyl, azide, isothiocyanate, alkyl, aralkyl, alkenyl, alkynyl or protected alkynyl, alkoxyl, arylcarbonyl, cycloalkyl, formyl, haloalkyl, hydroxyl, amino, nitro, sulfhydryl, amido, phosphonate, phosphinate, alkylthio, sulfonyl, sulfonamido, heterocyclyl, aryl, heteroaryl, —CF₃, —CF₂R⁷, —CFR⁷₂, —CN, polyethylene glycol, polyethylene imine, or —(CH₂)ₚ-FG-R⁷;
p is independently for each occurrence an integer from 0-10;
FG is independently for each occurrence selected from the group consisting of C(O), CO₂, O(CO), C(O)NR⁷, NR⁷C(O), O, Si(R⁷)₂, C(NR⁷), (R⁷)₂N(CO)N(R⁷)₂, OC(O)NR⁷, NR⁷C(O)O, and C(N═N);
R⁷ is independently for each occurrence selected from the group consisting of H, alkyl, cycloalkyl, aryl, aralkyl, alkenyl, and alkynyl; and
if two or more substituents are present on Ar¹, then two of said substituents taken together may form a ring.

14. The method of claim 1, wherein Ar¹ is covalently linked to a fluorophore, an imaging agent, a detection agent, a biomolecule, a therapeutic agent, a lipophilic moiety, a member of a high-affinity binding pair, or a cell-receptor targeting agent.

15. The method of claim 14, wherein Ar¹ is covalently linked to biotin.

16. The method of claim 14, wherein Ar¹ is covalently linked to fluorescein.

17. (canceled)

18. The method of claim 1, wherein Ar¹ is comprises a fluorophore.

19. The method of claim 1, wherein Ar¹ is comprises a therapeutic agent.

20. The method of claim 19, wherein the therapeutic agent is trametinib, topotecan, abiraterone, dabrafenib, or vandetanib.

21. The method of claim 1, wherein A¹ and A² are independently a natural or unnatural amino acid, a plurality of natural or unnatural amino acids, a peptide, an oligopeptide, a polypeptide, or a protein.

22. The method of claim 1, wherein A¹ comprises arginine, histidine, lysine, aspartic acid, glutamic acid, serine, threonine, asparagine, glutamine, proline, tyrosine, or tryptophan.

23. The method of claim 1, wherein A² comprises arginine, histidine, lysine, aspartic acid, glutamic acid, serine, threonine, asparagine, glutamine, proline, tyrosine, or tryptophan.

24. The method of claim 1, wherein A¹ and A² do not comprise cysteine or selenocysteine.

25. The method of claim 1, wherein the limiting reagent is H

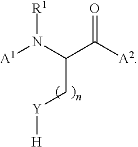

26. The method of claim 1, wherein when A¹ or A² comprises an —SH or —SeH moiety; and the molar ratio of the amount of 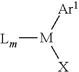
to the amount of 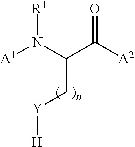
multiplied by the aggregate number of —SH and —SeH moieties in 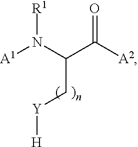
is greater than 1:1.

27-30. (canceled)

31. The method of claim 1, wherein A¹ and A² are covalently linked.

32-123. (canceled)
